(19) United States
(12) Patent Application Publication
(10) Pub. No.: US 2004/0220403 A1
(43) Pub. Date: Nov. 4, 2004

(54) 1-(2-Methoxybenzyl)-3-benzhydrylpiperazines as tachykinin anatgonists

(76) Inventors: Kazuhiko Take, Osaka-shi (JP); Chiyoshi Kasahara, Osaka (JP); Shinji Shigenaga, Osaka (JP); Hidenori Azami, Osaka (JP); Yoshiteru Eikyu, Osaka (JP); Kazuo Nakai, Osaka (JP); Masataka Morita, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/451,365

(22) PCT Filed: Dec. 21, 2001
(86) PCT No.: PCT/JP01/11240

(30) Foreign Application Priority Data
Jan. 2, 2001 (AU) PR2373

(51) Int. Cl. C07D 413/00

(52) U.S. Cl. 544/120

(57) ABSTRACT

A compound of the formula (I): wherein in which R¹, R², R³, R⁴, R⁵, R⁶, R⁷, R⁸, R⁹, R¹⁰ and R¹¹ are each as defined in the description, or a salt thereof. The object compound of the present invention has pharmacological activities such as Tachykinin antagonism and is useful for manufacture of a medicament for treating or preventing Tachykinin-mediated diseases.

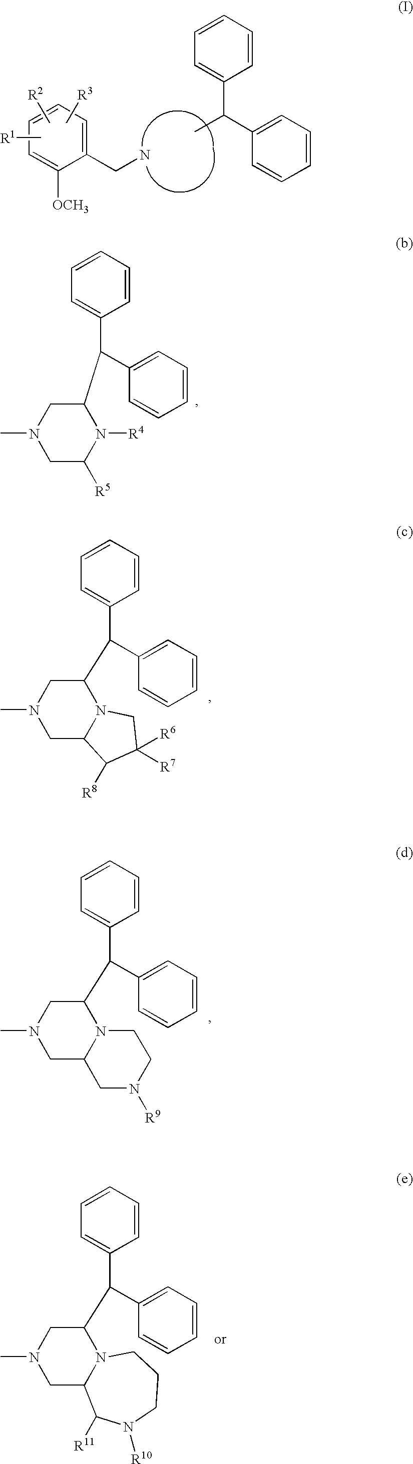

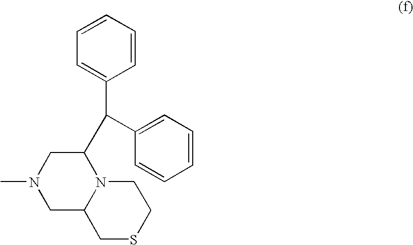

### TECHNICAL FIELD

The present invention relates to new benzhydryl derivatives and a salt thereof.

More particularly, it relates to new benzhydryl derivatives and a salt thereof which have pharmacological activities such as Tachykinin antagonism, especially Substance P antagonism, Neurokinin A antagonism, Neurokinin B antagonism, and the like, to a process for preparation thereof, to a pharmaceutical composition comprising the same, and to a use of the same as a medicament.

Accordingly, one object of the present invention is to provide new and useful benzhydryl derivatives and a salt thereof which have pharmacological activities such as Tachykinin antagonism, especially Substance P antagonism, Neurokinin A antagonism, Neurokinin B antagonism, and the like.

Another object of the present invention is to provide a process for the preparation of said benzhydryl derivatives and a salt thereof.

A further object of the present invention is to provide a pharmaceutical composition comprising, as an active ingredient, said benzhydryl derivatives and a pharmaceutically acceptable salt thereof.

Still further object of the present invention is to provide a use of said benzhydryl derivatives or a pharmaceutically acceptable salt thereof as Tachykinin antagonist, especially Substance P antagonist, Neurokinin A antagonist or Neurokinin B antagonist, useful for treating or preventing Tachykinin-mediated diseases, for example, respiratory diseases such as asthma, bronchitis, rhinitis, cough, expectoration, and the like; ophthalmic diseases such as conjunctivitis, vernal conjunctivitis, and the like; cutaneous diseases such as contact dermatitis, atopic dermatitis, urticaria, and other eczematoid dermatitis, and the like; inflammatory diseases such as rheumatoid arthritis, osteoarthritis, and the like; pains or aches (e.g., migraine, headache, toothache, cancerous pain, back pain, etc.); and the like in human being or animals.

### DISCLOSURE OF INVENTION

The object compound of the present invention can be represented by the following general formula (I):

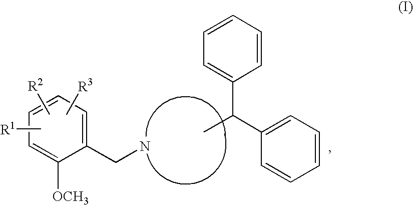

wherein

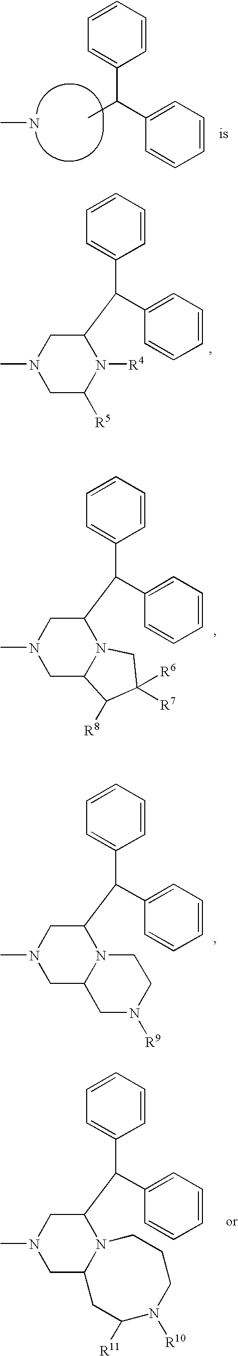

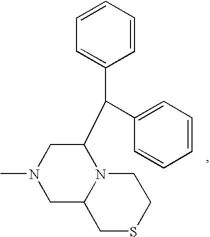

in which

R⁴ is hydrogen, lower alkanoyl or lower alkyl optionally substituted with carboxy, lower alkoxycarbonyl, pyridyl or lower alkylpyrazolyl,

R⁵ is hydrogen or lower alkoxycarbonyl,

R⁶ is hydrogen, halogen, oxo, hydroxy, lower alkanoyloxy, cyano, carbamoyl or amino optionally substituted with lower alkanoyl, hydroxy(lower)alkanoyl or benzyloxycarbonyl,

R⁷ is hydrogen or halogen,

R⁸ is hydrogen, oxo, lower alkanoyloxy, azido or amino optionally substituted with lower alkanoyl,

R⁹ is hydrogen; lower alkanoyl optionally substituted with hydroxy, carboxy, lower alkoxy, phenyl(lower)alkoxy, lower alkanoyloxy, lower alkoxycarbonyl, amino, lower alkanoylamino, benzyloxy(lower)alkanoylamino, hydroxy(lower)alkanoylamino, di(lower)alkylcarbamoyl or mono(or di or tri)halogen(s); cyclo(lower)alkylcarbonyl optionally substituted with hydroxy(lower)alkyl, amino or lower alkanoylamino; azetidinylcarbonyl; lower alkylimidazolylcarbonyl; pyridylcarbonyl; pyrimidinylcarbonyl; pyrazinylcarbonyl; lower alkyl optionally substituted with imino, cyclo(lower)alkyl, lower alkanoyl, lower alkoxycarbonyl, carbamoyl or di(lower)alkylcarbamoyl; cyclo(lower)alkyl; carbamoyl optionally substituted with mono(or di)(lower)alkyl(s); aminosulfonyl optionally substituted with mono(or di)(lower)alkyl(s); lower alkylsulfonyl optionally substituted with hydroxy, lower alkylsulfonyl or lower alkanoyloxy; or pyridyl,

R¹⁰ is hydrogen or lower alkanoyl,

R¹¹ is hydrogen or oxo, and

R¹, R² and R³ are independently hydrogen, halogen, lower alkyl, lower alkoxy or tetrazolyl optionally substituted with mono(or di or tri)halo(lower)alkyl.

It is to be noted that the object compound (I) may include one or more stereoisomers due to asymmetric carbon atom(s) and double bond, and all of such isomers and a mixture thereof are included within the scope of the present invention.

It is further to be noted that iscmerization or rearrangement of the object compound (I) may occur due to the effect of the light, acid, base or the like, and the compound obtained as the result of said isomerization or rearrangement is also included within the scope of the present invention.

It is also to be noted that the solvating form of the compound (I) (e.g. hydrate, etc.) and any form of the crystal of the compound (I) are included within the scope of the present invention.

According to the present invention, the object compound (I) or a salt thereof can be prepared by processes which are illustrated in the following schemes.

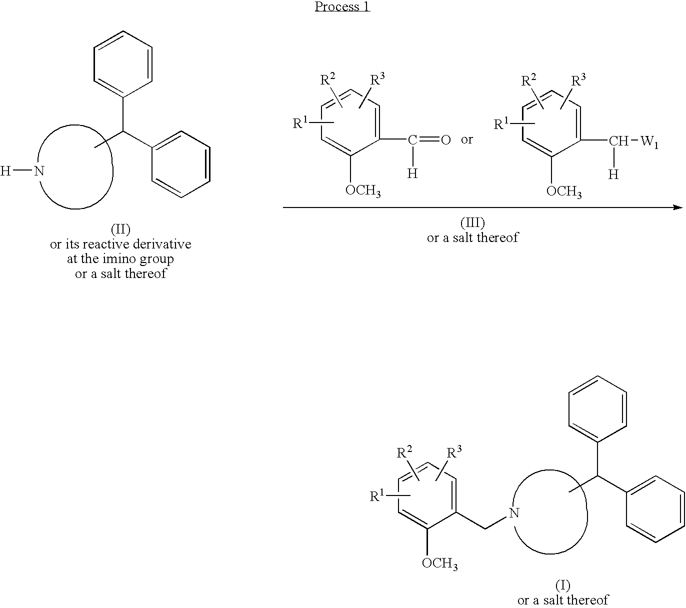

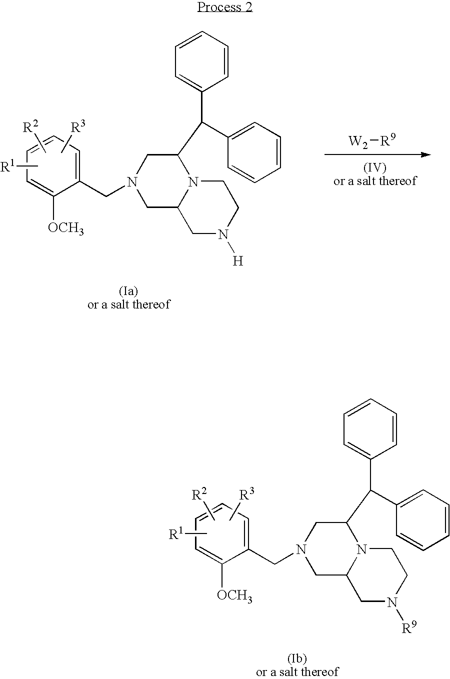

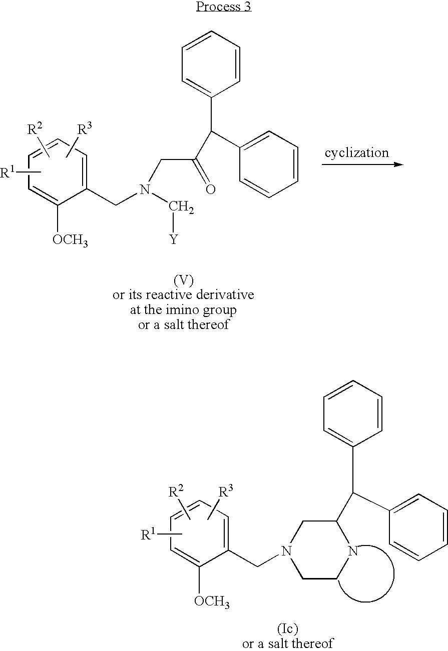

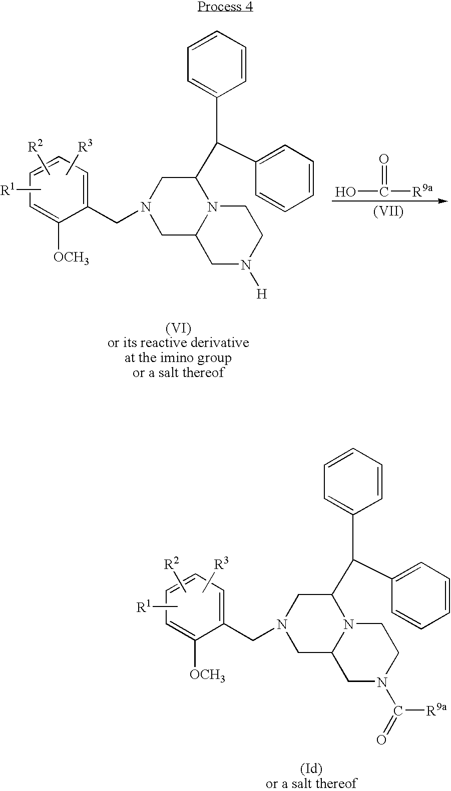

wherein

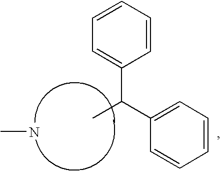

R¹, R², R³ and R⁹ are each as defined above,

R^{9a} is lower alkyl optionally substituted with hydroxy, carboxy, lower alkoxy, phenyl(lower)alkoxy, lower alkanoyloxy, lower alkoxycarbonyl, amino, lower alkanoylamino, benzyloxy(lower)alkanoylamino, hydroxy(lower)alkanoylamino, di(lower)alkylcarbamoyl or mono(or di or tri)halogen(s); cyclo(lower)alkyl optionally substituted with hydroxy(lower)alkyl, amino or lower alkanoylamino; azetidinyl; lower alkylimidazolyl; pyridyl; pyrimidinyl; or pyrazinyl,

W₁ and W₂ are each a leaving group,

Y is

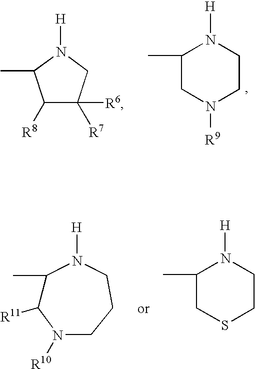

or its reactive derivative at the imino group [in which R⁶, R⁷, R⁸, R⁹, R¹⁰ and R¹¹ are each as defined above], and

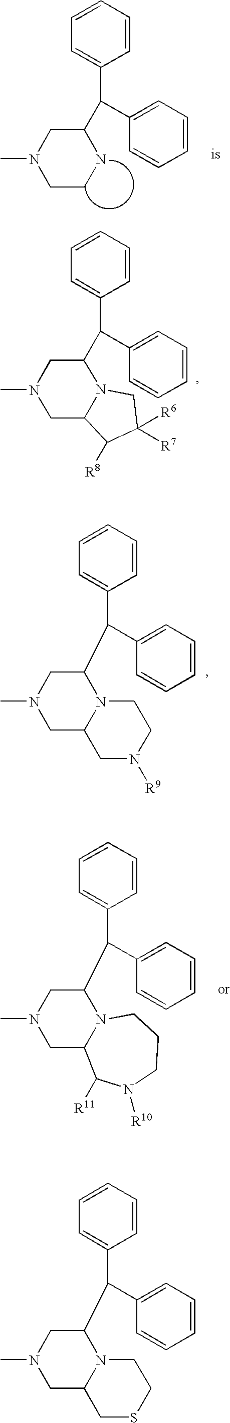

[in which R⁶, R⁷, R⁸, R⁹, R¹⁰ and R¹¹ are each as defined above].

As to the starting compounds (II), (III), (IV), (V), (VI) and (VII), some of them are novel and can be prepared by the procedures described in the Preparations and Examples mentioned later or similar manners thereto.

Suitable salts of the starting and object compounds are conventional non-toxic and pharmaceutically acceptable salt and include an acid addition salt such as an organic acid salt (e.g. acetate, trifluoroacetate, fumarate, maleate, tartrate, methanesulfonate, benzenesulfonate, formate, toluenesulfonate, etc.), an inorganic acid salt (e.g. hydrochloride, hydrobromide, hydroiodide, sulfate, nitrate, phosphate, etc.), or a salt with an amino acid (e.g. arginine, aspartic acid, glutamic acid, etc.), or a metal salt such as an alkali metal salt (e.g. sodium salt, potassium salt, etc.) and an alkaline earth metal salt (e.g. calcium salt, magnesium salt, etc.), an ammonium salt, an organic base salt (e.g. trimethylamine salt, triethylamine salt, pyridine salt, picoline salt, dicyclohexylamine salt, N,N′-dibenzylethylenediamine salt, etc.), or the like.

In the above and subsequent descriptions of the present specification, suitable examples and illustrations of the various definitions which the present invention intends to include within the scope thereof are explained in detail as follows.

The term “lower” is intended to mean 1 to 6, preferably 1 to 4, carbon atom(s), unless otherwise indicated.

Suitable “halogen” and “halogen” moiety in the term of “mono(or di or tri)halo(lower)alkyl” may include fluorine, chlorine, bromine and iodine.

Suitable “lower alkyl” and “lower alkyl” moiety in the terms of “mono(or di or tri)halo(lower)alkyl”, “lower alkylamino”, etc. may include straight or branched one having 1 to 6 carbon atom(s), such as methyl, ethyl, propyl, isopropyl, butyl, isobutyl, pentyl, hexyl and the like, in which the preferred one is C₁-C₄ alkyl and the most preferred one is methyl, ethyl or isopropyl.

Suitable “cyclo(lower)alkyl” and “cyclo(lower)alkyl” moiety in the term of “cyclo(lower)alkylcarbonyl” may include cyclopropyl, cyclobutyl, cyclopentyl, cyclohexyl and the like, in which the preferred one is cyclo(C₃-C₆)alkyl and the most preferred one is cyclopropyl or cyclobutyl.

Suitable “lower alkoxy” and “lower alkoxy” moiety in the term of “lower alkoxycarbonyl” may include methoxy, ethoxy, propoxy, isopropoxy, butoxy, isobutoxy, t-butoxy, pentyloxy, t-pentyloxy, hexyloxy and the like, in which the preferred one is C₁-C₄ alkoxy and the most preferred one is methoxy.

Suitable “lower alkanoyl” and “lower alkanoyl” moiety in the terms of “lower alkanoyloxy”, etc. may include formyl, acetyl, propanoyl, butanoyl, 2-methylpropanoyl, pentanoyl, 2,2-dimethylpropanoyl, hexanoyl and the like, in which the preferred one is C₁-C₄ alkanoyl and the most preferred one is acetyl, propoxy or 2-methylpropanoyl.

Suitable “leaving group” may include lower alkoxy (e.g. methoxy, ethoxy, propoxy, isopropoxy, butoxy, isobutoxy, t-butoxy, pentoxy, etc.), aryloxy (e.g., phenoxy, naphthoxy, etc.), an acid residue or the like.

Suitable “acid residue” may be halogen (e.g., chlorine, bromine, iodine, etc.), sulfonyloxy (e.g., methanesulfonyloxy, phenylsulfonyloxy, mesitylenesulfonyloxy, toluenesulfonyloxy, etc.) or the like.

Preferred embodiments of the object compound (I) are as follows:

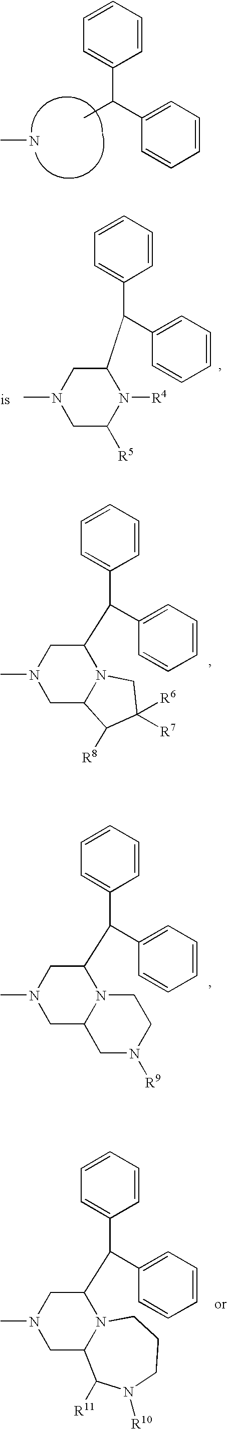

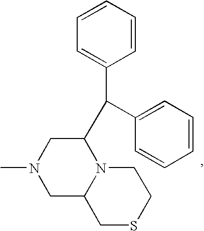

In which

R⁴ is lower alkanoyl or lower alkyl optionally substituted with carboxy, lower alkoxycarbonyl, pyridyl or lower alkylpyrazolyl,

R⁵ is hydrogen,

R⁶ is halogen, oxo, hydroxy, lower alkanoyloxy, cyano, carbamoyl or amino optionally substituted with lower alkanoyl, hydroxy(lower)alkanoyl or benzyloxycarbonyl,

R⁷ is hydrogen,

R⁸ is hydrogen,

R⁹ is lower alkanoyl substituted with hydroxy, carboxy, lower alkoxy, phenyl(lower)alkoxy, lower alkanoyloxy, lower alkoxycarbonyl, amino, lower alkanoylamino, benzyloxy(lower)alkanoylamino, hydroxy(lower)alkanoylamino, di(lower)alkylcarbamoyl or mono(or di or tri)halogen(s); cyclo(lower)alkylcarbonyl optionally substituted with hydroxy(lower)alkyl, amino or lower alkanoylamino; azetidinylcarbonyl; lower alkylimidazolylcarbonyl; pyridylcarbonyl; pyrimidinylcarbonyl; pyrazinylcarbonyl; lower alkyl optionally substituted with imino, cyclo(lower)alkyl, lower alkanoyl, lower alkoxycarbonyl, carbamoyl or di(lower)alkylcarbamoyl; cyclo(lower)alkyl; carbamoyl optionally substituted with mono(or di)(lower)alkyl(s); aminosulfonyl optionally substituted with mono(or di)(lower)alkyl(s); lower alkylsulfonyl optionally substituted with hydroxy, lower alkylsulfonyl or lower alkanoyloxy; or pyridyl,

R¹⁰ is hydrogen or lower alkanoyl,

R¹¹ is hydrogen or oxo, and

R¹, R² and R³ are independently hydrogen, halogen, lower alkyl, lower alkoxy or tetrazolyl optionally substituted with mono(or di or tri)halo(lower)alkyl.

More preferred embodiments of the object compound (I) are as follows:

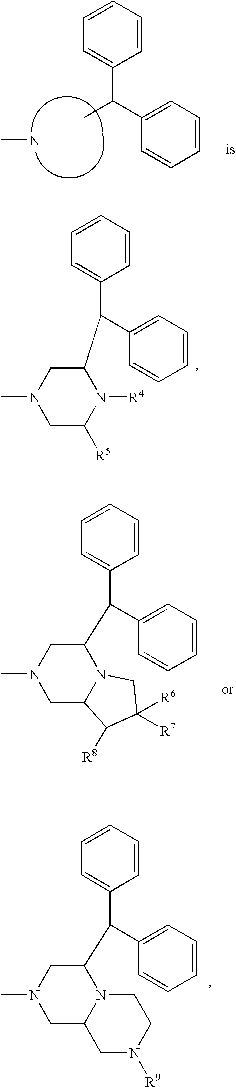

in which

R⁴ is lower alkanoyl or lower alkyl optionally substituted with carboxy, lower alkoxycarbonyl, pyridyl or lower alkylpyrazolyl,

R⁵ is hydrogen,

R⁶ is halogen, oxo, hydroxy, lower alkanoyloxy, cyano, carbamoyl or amino optionally substituted with lower alkanoyl, hydroxy(lower)alkanoyl or benzyloxycarbonyl,

R⁷ is hydrogen,

R⁸ is hydrogen,

R⁹ is lower alkanoyl substituted with hydroxy, carboxy, lower alkoxy, phenyl(lower)alkoxy, lower alkanoyloxy, lower alkoxycarbonyl, amino, lower alkanoylamino, benzyloxy(lower)alkanoylamino, hydroxy(lower)alkanoylamino, di(lower)alkylcarbamoyl or mono(or di or tri)halogen(s); cyclo(lower)alkylcarbonyl optionally substituted with hydroxy(lower)alkyl, amino or lower alkanoylamino; azetidinylcarbonyl; lower alkylimidazolylcarbonyl; pyridylcarbonyl; pyrimidinylcarbonyl; pyrazinylcarbonyl; lower alkyl optionally substituted with imino, cyclo(lower)alkyl, lower alkanoyl, lower alkoxycarbonyl, carbamoyl or di(lower)alkylcarbamoyl; cyclo(lower)alkyl; carbamoyl optionally substituted with mono(or di)(lower)alkyl(s); aminosulfonyl optionally substituted with mono (or di)(lower)alkyl(s); lower alkylsulfonyl optionally substituted with hydroxy, lower alkylsulfonyl or lower alkanoyloxy; or pyridyl, and

R¹, R² and R³ are independently hydrogen, lower alkoxy or tetrazolyl substituted with mono(or di or tri)halo(lower)alkyl.

The Processes 1, 2, 3 and 4 for preparing the object compound (I) of the present invention are explained in detail in the following.

Process 1

The object compound (I) or a salt thereof can be prepared by reacting the compound (II) or its reactive derivative at the imino group or a salt thereof with the compound (III) or a salt thereof.

Suitable reactive derivative at the imino group of the compound (II) may include Schiff's base type imino or its tautomeric enamine type isomer formed by the reaction of the compound (II) with a carbonyl compound such as aldehyde, ketone or the like; a silyl derivative formed by the reaction of the compound (II) with a silyl compound such as bis (trimethylsilyl) acetamide, mono(trimethylsilyl)acetamide, bis(trimethylsilyl)urea or the like; a derivative formed by reaction of the compound (II) with phosphorus trichloride or phosgene and the like.

The reaction is usually carried out in a conventional solvent such as water, alcohol (e.g. methanol, ethanol, etc.), acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction, or the mixture thereof.

The reaction may also be carried out in the presence of a reductive regent such as hydrides (e.g. hydrogen iodide, hydrogen sulfide, lithium aluminum hydride, sodium borohydride, sodium cyanoborohydride, sodium triacetoxyborohydride, etc.), or the like.

The reaction temperature is not critical, and the reaction is usually carried out under cooling to heating.

Process 2

The object compound (Ib) or a salt thereof can be prepared by reacting the compound (Ia) or a salt thereof with the compound (IV) or a salt thereof.

The reaction is usually carried out in a conventional solvent such as water, alcohol (e.g. methanol, ethanol, etc.), acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction. These conventional solvents may also be used in a mixture with water.

The reaction may also be carried out in the presence of an inorganic or organic base such as alkali metal carbonate (e.g. potassium carbonate, etc.), alkali metal bicarbonate, tri(lower)alkylamine, pyridine, N-(lower)alkyl-morpholine, N,N-di(lower)alkylethylamine (e.g. N,N-diisopropylethylamine, etc.), N,N-di(lower)alkylbenzylamine, or the like.

The reaction temperature is not critical, and the reaction is usually carried out under cooling to heating.

Process 3

The object compound (Ic) or a salt thereof can be prepared by cyclizing the compound (V) or its reactive derivative at the imino group or a salt thereof.

This reaction can be carried out in substantially the same manner as in Preparation 32.

Process 4

The object compound (Id) or a salt thereof can be prepared by reacting the compound (VI) or its reactive derivative at the imino group or a salt thereof with the compound (VII).

This reaction can be carried out in substantially the same manner as in Example 17.

The object compound (I) and a pharmaceutically acceptable salt thereof have pharmacological activities such as Tachykinin antagonism, especially Substance P antagonism, Neurokinin A antagonism or Neurokinin B antagonism, and therefore are useful for treating or preventing Tachykinin-mediated diseases, particularly Substance P-mediated diseases, for example, respiratory diseases such as asthma, bronchitis (e.g. chronic bronchitis, acute bronchitis and diffuse panbronchiolitis, etc.), rhinitis, cough, expectoration, and the like; ophthalmic diseases such as conjunctivitis, vernal conjunctivitis, and the like; cutaneous diseases such as contact dermatitis, atopic dermatitis, urticaria, and other eczematoid dermatitis, and the like; inflammatory diseases such as rheumatoid arthritis, osteoarthritis, and the like; pains or aches (e.g. migraine, headache, cluster headache, toothache, cancerous pain, back pain, neuralgia, etc.); and the like.

Further, it is expected that the object compound (I) and a pharmaceutically acceptable salt thereof of the present invention are useful for treating or preventing ophthalmic diseases such as glaucoma, uveitis, and the like; gastrointestinal diseases such as ulcer, ulcerative colitis, irritable bowel syndrome, food allergy, and the like; inflammatory diseases such as nephritis, and the like; circulatory diseases such as hypertension, angina pectoris, cardiac failure, thrombosis, Raynaud's disease, and the like; epilepsy; spastic paralysis; pollakiuria; cystitis; bladder detrusor hyperreflexia; urinary incontinence; Parkinson diseases; dimentia; AIDS related dementia; Alzheimer's diseases; Down's syndrome; Huntington's chorea; carcinoid syndrome; disorders related to immune enhancement or suppression; disorders caused by Helicobacter pylori or another spiral urease-positive gram-negative bacterium; sunburn; angiogenesis or diseases caused by angiogenesis; and the like.

It is furthermore expected that the object compound (I) and a pharmaceutically acceptable salt thereof of the present invention are useful for treating or preventing chronic obstructive pulmonary diseases, particularly chronic pulmonary emphysema; iritis; proliferative vitreoretinopathy; psoriasis; inflammatory intestinal diseases, particularly Crohn's diseases; hepatitis; superficial pain on congelation, burn, herpes zoster or diabetic neuropathy; telalgia attended to hyperlipidemia; postoperative neuroma, particularly of mastectomy; vulvar vestibulitis; hemodialysis-associated itching; lichen planus; laryngopharyngitis; bronchiectasis; coniosis; whooping cough; pulmonary tuberculosis; cystic fibrosis; emesis (e.g., nausea, retching, vomiting, acute emesis, delayed emesis, anticipatory emesis, past operative nausea and vomiting (PONV), acute and/or delayed emesis induced by drugs such as cancer chemotherapeutic agents, etc.); mental diseases, particularly anxiety disorders, stress-related disorders, affective disorders, psychological development disorders and schizophrenia; demyelinating diseases such as multiple sclerosis and amyotrophic lateral sclerosis; attenuation of morphine withdrawal; oedema, such as oedema caused by thermal injury; small cell carcinomas, particularly small cell lung cancer (SCLC); hypersensitivity disorders such as poison ivy; fibrosing and collagen diseases such as scleroderma and eosinophilic fascioliasis; reflex sympathetic dystrophy such as shoulder/hand syndrome; addiction disorders such as alcoholism; stress related somatic disorders; rheumatic diseases such as fibrositis; aggressive behaviour, optionally taking an antipsychotic agent together; mania or hypomania, optionally taking an antipsychotic agent together; symptoms associated with Premenstrual Syndrome (PMS) (PMS is also now referred to as Late Luteal Phase Syndrome (LLS); psychosomatic disoredrs; psychoimmunologic disoredrs; attetion deficit disoredrs (ADD) with or without hyperactivity; and the like.

Furthermore, the object compound (I) and a pharmaceutically acceptable salt thereof of the present invention are Central Nervous System (CNS) penetrant.

For therapeutic purpose, the compound (I) and a pharmaceutically acceptable salt thereof of the present invention can be used in a form of pharmaceutical preparation containing one of said compounds, as an active ingredient, in admixture with a pharmaceutically acceptable carrier such as an organic or inorganic solid or liquid excipient suitable for oral, parenteral, external including topical, internal, intravenous, intramuscular, inhalant, nasal, intraarticular, intraspinal, transtracheal or transocular administration. The pharmaceutical preparations may be solid, semi-solid or solutions such as capsules, tablets, pellets, dragees, powders, granules, suppositories, ointments, creams, lotions, inhalants, injections, cataplasms, gels, tapes, eye drops, solution, syrups, aerosols, suspension, emulsion, or the like. If desired, there may be included in these preparations, auxiliary substances, stabilizing agents, wetting or eulsifying agents, buffers and other commonly used additives.

While the dosage of the compound (I) will vary depending upon the age and condition of a patient, an average single dose of about 0.1 mg, 1 mg, 10 mg, 50 mg, 100 mg, 250 mg, 500 mg and 1000 mg of the compound (I) may be effective for treating Tachykinin-mediated diseases such as asthma and the like. In general, amounts between 0.1 mg/body and about 1,000 mg/body may be administered per day.

In order to show the utility of the object compound (I) and a pharmaceutically acceptable salt thereof, the pharmacological test data of some representative compounds of the present invention is shown in the following.

Emesis in the Dog

[I] Test Method

Individually housed adult female dogs (8 to 15 kg) were given an i.v. injection of a solution containing a test compound. 5 Min later the emetic responses (retching and vomiting) were induced by administration of subcutaneous apomorphine (0.1 mg/0.5 ml/kg) and observed for the next 60 min. The timing and number of retches and vomits observed were recorded for each animal. An individual animal was tested with at least 10 days between experiments.

[II] Test Result

The following Test Compounds showed 100% inhibition rate of emesis in the dog at the dose of 1.0 mg/kg.

Test compound: The object compounds of the

Examples 52-(5), 52-(14) and 57-(1)

The following Preparations and Examples are given for the purpose of illustrating this invention.

Preparation 1

4N Hydrogen chloride in 1,4-dioxane (44 ml) was added to a solution of 4-tert-butoxycarbonyl-2-benzhydryl-1-methylpiperazine (6.5 g) in ethanol (33 ml) under ice-cooling over 30 minutes. The mixture was stirred at room temperature for 4 hours and evaporated under reduced pressure. The residue was triturated with diisopropyl ether and the resulting solid was collected by filtration to give 2-benzhydrylpiperazine dihydrochloride (6.02 g) as a powder.

NMR (DMSO-d₆, δ): 2.50-3.95 (6H, m), 3.56 (3H, s), 4.30-5.50 (2H, m), 7.21-7.57 (11H, m) MASS (APCI): 267 (M+H)⁺ (free)

Preparation 2

3-Bromo-1,1-diphenyl-2-propanone (12.7 g) and N,N-diisopropylethylamine (15.7 ml) were added successively to a solution of (2S)-2-[(2-methoxybenzylamino) methyl]-pyrrolidine-1-carboxylic acid benzyl ester (15.6 g) in tetrahydrofuran (156 ml) at 0° C. After being stirred at room temperature for 2 hours, the mixture was poured into ice-water (100 ml) and extracted with ethyl acetate (100 ml×2). The extract was washed with brine, dried over magnesium sulfate and evaporated under reduced pressure. The residue was purified by column chromatography on silica gel using a mixed solvent of hexane and ethyl acetate (3:1). The fractions containing the objective compound were collected and evaporated under reduced pressure to give a colorless syrup of (2S)-2-[[N-(2-oxo-3,3-diphenylpropyl)-N-(2-methoxybenzyl)amino]methyl]pyrrolidine-1-carboxylic acid benzyl ester (1.51 g).

NMR (CDCl₃, δ): 1.30-2.00 (3H, m), 2.23-2.70 (2H, m), 3.11-3.93 (8H, m), 3.74 (3H, 8), 5.06 (2H, m), 5.36 (1H, m), 6.82-7.31 (19H, m) MASS (APCI): 563 (M+H)⁺

Preparation 3

A solution of dimethyl sulfoxide (0.219 ml) in dichloromethane (1.1 ml) was added dropwise to a solution of oxalyl chloride (0.133 ml) in dichloromethane (2.7 ml) under cooling below −60° C. with dry ice-acetone. After 5 minutes, the mixture was allowed to −10° C., and a solution of (2S)-1-benzyl-2-(hydroxymethyl)piperidine (156.5 mg) in dichloromethane (1.6 ml) was added to the mixture. The whole mixture was then cooled below −60° C. and was stirred for 20 minutes at the same temperature. After addition of triethylamine (0.64 ml) followed by stirring at room temperature, the reaction mixture was poured into water and extracted with 1,2-dichloroethane. The extract was dried over magnesium sulfate and evaporated under reduced pressure to give a syrup. Benzylamine (0.33 ml) was added to the solution of the syrup obtained above procedure in 1,2-dichloroethane (2.5 ml) with ice-cooling. After the whole was stirred for 30 minutes at the same temperature, sodium triacetoxyborohydride (0.323 g) was added to this mixture. The reaction mixture was allowed to room temperature and was stirred for 3 hours. The mixture was poured into aqueous saturated sodium hydrogen carbonate solution and extracted with dichloromethane. The extract was dried over magnesium sulfate and evaporated under reduced pressure. The resulting residue was purified by silica gel chromatography using a mixture of dichloromethane and methanol (20:1) as an eluent to give N-benzyl-[(2S)-1-benzylpiperidin-2-ylmethyl]amine (168.5 mg).

NMR (CDCl₃, δ): 1.26-1.49 (3H, m), 1.56-1.67 (3H, m), 2.03 (1H, s), 2.04-2.14 (1H, m), 2.42-2.50 (1H, m), 2.66-2.86 (3H, m), 3.25 (1H, d, J=13.6 Hz), 3.73 (2H, s), 3.92 (1H, d, J=13.6 Hz), 7.19-7.38 (20H, m) MASS (APCI): 295 (M+H)⁺

Preparation 4

1-[3-(Dimethylamino)propyl]-3-ethylcarbodiimide hydrochloride (2.11 g) was added over 5 minutes to a mixture of N,O-dimethylhydroxylamine hydrochloride (1.17 g), (2S)-piperazine-1,2,4-tricarboxylic acid 4-benzyl ester 1-tert-butyl ester (3.64 g), 1-hydroxybenzotriazole (1.49 g) and N,N-diisopropylethylamine (2.1 ml) in dichloromethane (40 ml). After being stirred for 18 hours at room temperature, the resulting mixture was extracted with ethyl acetate. The extract was washed with brine, dried over sodium sulfate and evaporated under reduced pressure. The residue was purified by column chromatography on silica gel using a mixed solvent of hexane and ethyl acetate (3:1) to give 2-(N-methoxy-N-methylcarbamoyl)piperazine-1,4-dicarboxylic acid 4-benzyl ester 1-tert-butyl ester (3.61 g) as a colorless powder.

NMR (CDCl₃, δ): 1.45 (9H, s), 2.90-3.20 (5H, m), 3.60-4.20 (6H, m), 4.41 (1H, m), 4.90 (1H, m), 5.06 (1H, d, J=12.4 Hz), 5.16 (1H, d, J=12.4 Hz), 7.33 (5H, m) MASS (APCI): 308 (M-Boc+H)⁺

Preparation 5

Lithium aluminum hydride (38 mg) was added by small portions to an ice-cooled solution of 2-(N-methoxy-N-methylcarbamoyl)piperazine-1,4-dicarboxylic acid 4-benzyl ester 1-tert-butyl ester (407 mg) in tetrahydrofuran (5 ml) below 5° C. under nitrogen atmosphere. After the mixture was stirred at the same temperature for 2.5 hours, 2N sodium hydroxide (0.2 ml) was added to the mixture. After the mixture was stirred for 30 minutes, the insoluble materials were removed by filtration and washed with tetrahydrofuran. The filtrate and the washing were combined, and evaporated under reduced pressure to give a residue. Sodium triacetoxyborohydride (424 mg) was added portionwisely to a stirred mixture of the residue obtained in the above procedure and 2-methoxybenzylamine (151 mg) in dichloromethane (4 ml). After being stirred at room temperature for 4 hours, 3-bromo-1,1-diphenyl-2-propanone (347 mg) in N,N-dimethylformamide (5 ml) and N,N-diisopropylethylamine (0.35 ml) were added successively to the reaction mixture at 5° C. The whole mixture was stirred at room temperature for 36 hours and then poured into ice-water, and extracted with ethyl acetate. The extract was washed with brine, dried over magnesium sulfate and evaporated under reduced pressure. The residue was purified by column chromatography on silica gel using a mixed solvent of hexane and ethyl acetate (4:1) to give (2R)-2-[[N-(2-methoxybenzyl)-N-(2-oxo-3,3-diphenylpropyl)-amino]methyl]piperazine-1,4-dicarboxylic acid 4-benzyl ester 1-tert-butyl ester (170 mg) as a colorless powder.

NMR (CDCl₃, δ): 1.41-1.57 (9H, m), 2.70-3.00 (5H m), 3.25-4.35 (11H, m), 4.95-5.15 (3H, m), 6.70-7.29 (19H, m)

Preparation 6

Methanesulfonyl chloride (0.18 ml) was added dropwise to an ice-cooled solution of tert-butyl (4R, 7R, 8aS)-4-benzhydryl-7-hydroxyhexahydropyrrolo[1,2-a]pyrazine-2(1H)-carboxylate (0.78 g) and triethylamine (0.53 ml) in dichlorometane. After being stirred for 3 hours at the same temperature the mixture was washed with aqueous saturated sodium hydrogen carbonate, dried over magnesium sulfate and concentrated under reduced pressure. The syrup obtained by above procedure and sodium azide (126 mg) was dissolved into dimethylsulfoxide (5 ml). The whole was stirred at 75° C. for 15 hours. The mixture was poured into water and extracted with ethyl acetate. The extract was washed with brine, dried over magnesium sulfate and concentrated under reduced pressure. The syrup was purified by column chromatography on silica gel using a mixed solvent of hexane and ethyl acetate (30:1). The fractions containing the objective compound were collected to give (4R,7S,8aS)-4-benzhydryl-2-(tert-butoxycarbonyl)octahydropyrrolo[1,2-a]pyrazine-7-azide (0.70 mg).

NMR (CDCl₃, δ): 1.30-1.40 (2H, m), 1.38 (9H, s), 1.98-2.06 (1H, m), 2.15-2.27 (2H, m), 2.31-2.65 (2H, m), 2.78 (1H, d, J=8.6 Hz), 3.00-3.20 (1H, m), 3.63-3.72 (2H, m), 4.04 (1H, d, J=8.7 Hz), 7.13-7.43 (10H, m) MASS (APCI): 434 (M+H)⁺ (free)

Preparation 7

Acetic anhydride (25.3 μl) was added to an ice cooled solution of tert-butyl (4R,7S,8aS)-7-amino-4-benzhydrylhexahydropyrrolo[1,2-a]pyrazine-2(1H)-carboxylate (0.1 g) and pyridine (0.096 ml) in dichloromethane (1 ml). After being stirred for 2 hours at the same temperature the mixture was poured into aqueous sodium hydrogen carbonate and extracted with dichloromethane. The organic layer was separated, dried over magnesium sulfate, concentrated under reduced pressure. The syrup was purified by column chromatography on silica gel using a mixed solvent of hexane and ethyl acetate (4:1). The fractions containing the objective compound were collected to give tert-butyl (4R,7S,8aS)-7-(acetylamino)-4-benzhydrylhexahydropyrrolo(1,2-a]pyrazine-2(1H)-carboxylate (110 mg) as a syrup.

MASS (APCI): 450 (M+H)⁺

Preparation 8

1,1,1-Triacetoxy-1,1-dihydro-1,2-benziodoxol-3 (1H)-one (Dess-Martin periodinane) (159 mg) was added into a solution of tert-butyl (4R,7R,8aS)-4-benzhydryl-7-hydroxyhexahydropyrrolo[1,2-a]pyrazine-2(1H)-carboxylate (102.4 mg) in dichloromethane (1.5 ml) under ice-cooling. After being stirred for 1 hour at the same temperature, the reaction mixture was stirred for 2 hours at room temperature. Then the reaction mixture was poured into saturated aqueous sodium thiosulfate (5 ml), and the whole was stirred for 30 minutes. The aqueous mixture was extracted with dichloromethane. The extracts were washed with brine, dried over magnesium sulfate and evaporated under reduced pressure. The resulting residue was purified by silica gel column chromatography with a mixture of hexane and ethyl acetate (1:1) as an eluent. The fractions containing the objective compound were collected to give tert-butyl (4R,8aS)-4-benzhydryl-7-oxohexahydropyrrolo[1,2-a]pyrazine-2 (1H)-carboxylate (76.5 mg).

NMR (CDCl₃, δ): 1.39 (9H, s), 1.96-2.09 (1H, m), 2.28-2.48 (2H, m), 2.60-2.71 (2H, m), 2.88 (1H, m), 3.12 (1H, d, J=17 Hz), 3.41 (1H, m), 3.92-4.14 (3H, m), 7.16-7.39 (10H, m) MASS (APCI): 407 (M+H)⁺

Preparation 9

(Diethylamino)sulfur trifluoride (124 mg) was added into a solution of tert-butyl (4R,8aS)-4-benzhydryl-7-oxohexahydropyrrolo[1,2-a]pyrazine-2(1H)-carboxylate (69.5 mg) in 1,2-dichloroethane (1.5 ml) at room temperature. After being stirred for 2 days at the same temperature, the reaction mixture was poured into aqueous saturated sodium hydrogen cabonate. The aqueous layer was extracted with dichloromethane. The combined extracts were washed with brine, dried over magnesium sulfate and evaporated under reduced pressure. The resulting residue was purified by silica gel column chromatography with a mixture of hexane and ethyl acetate (2:1) as an eluent to give tert-butyl (4R,8aS)-4-benzhydryl-7,7-difluorohexahydropyrrolo[1,2-a]pyrazine-2(1H)-carboxylate (37.2 mg).

NMR (CDCl₃, δ): 1.39 (9H, s), 2.04-4.18 (11H, m), 6.73-7.78 (10H, m) MASS (APCI): 429 (M+H)⁺

Preparation 10

Triphenylphosphine (860 mg), acetic acid (159 mg) and diisopropyl azodicarboxylate were added successively into a solution of tert-butyl (4R,7R,8aS)-4-benzhydryl-7-hydroxyhexahydropyrrolo[1,2-a]pyrazine-2(1H)-carboxylate (670 mg) in tetrahydrofuran (10 ml) at room temperature. After being stirred for 1 hour at room temperature, the reaction mixture was poured into aqueous saturated sodium hydrogen carbonate. The whole was extracted with ethyl acetate. The extract was washed with brine, dried over magnesium sulfate and evaporated under reduced pressure. The resulting residue was purified by silica gel column chromatography with a mixture of hexane and ethyl acetate (2:1-3:2) as an eluent to give tert-butyl (4R,7S,8aS)-7-acetoxy-4-benzhydrylhexahydropyrrolo[1,2-a]pyrazine-2(1H)-carboxylate.

NMR (CDCl₃, δ): 1.30-1.43 (11H, m), 2.01-2.04 (3H, m), 2.08-2.79 (6H, m), 3.12 (1H, m), 3.77-4.10 (2H, m), 4.89-5.01 (1H, m), 6.71-7.42 (10H, m) MASS (APCI): 451 (M+H)⁺

Preparation 11

The following compound was obtained according to a similar manner to that of Preparation 6.

### tert-Butyl (4R, 7R, 8aS)-7-azido-4-benzhydrylhexahydropyrrolo (1, 2-a]pyrazine-2 (1H)-carboxylate

NMR (CDCl₃, δ): 1.38 (9H, s), 1.64-1.91 (3H, m), 2.40-2.60 (3H, m), 3.05-3.24 (2H, m), 3.82-4.18 (4H, m), 7.15-7.41 (10H, m) MASS (APCI): 433 M+H)⁺

Preparation 12

The following compound was obtained according to a similar manner to that of Preparation 7 from tert-butyl (4R,7R,8aS)-7-amino-4-benzhydrylhexahydropyrrolo (1,2-a]pyrazine-2 (1H)-carboxylate.

### tert-Butyl (4R, 7R, 8aS)-7-(acetylamino)-4-benzhydrylhexahydropyrrolo[1,2-a]pyrazine-2 (1H)-carboxylate

NMR (CDCl₃, δ): 1.37 (9H, s), 1.71 (5H, m), 2.03 (1H, dd, J=3.3, 7.3 Hz), 2.46-2.61 (2H, m), 2.94-3.07 (2H, m), 3.54-3.90 (4H, m), 4.21-4.28 (1H, m), 5.13-5.17 (1H, m), 7.15-7.42 (10H, m)

MASS (APCI): 450 (M+H)⁺

Preparation 13

To a solution of tert-butyl (4R,7R,8aS)-4-benzhydryl-7-hydroxyhexahydropyrrolo[1,2-a]pyrazine-2 (1H)-carboxylate (300 mg) in dichloromethane (2.5 ml) were added triethylamine (0.154 ml) and mesylchloride (68.2 μl) at 0° C. After stirring at 0° C. for 1 hour, the mixture was quenched with water and extracted with ethyl acetate (×3). The combined extracts were washed with brine, dried over magnesium sulfate, and evaporated under reduced pressure to give 355.5 mg of solid. The mixture of the solid and sodium cyanide (150 mg) in dimethyl sulfoxide (2.5 ml) was stirred at 70° C. for 5 hours.

Then, to the mixture was added sodium cyanide (396 mg). After stirring at 90° C. overnight, the mixture was quenched with water (100 ml) and extracted with ethyl acetate (×4). The combined extracts were washed with brine, dried over magnesium sulfate, and evaporated under reduced pressure. The residue was purified with preparative TLC (ethyl acetate/hexane=1/1) to give tert-butyl (4R,7S,8aS)-4-benzhydryl-7-cyanohexahydropyrrolo[1,2-a]pyrazine-2(1H)-carboxylate (205 mg) as an oil.

IR (KBr): 2241, 1693 cm⁻¹ NMR (CDCl₃, δ): 1.38 (9H, s), 1.60-1.72 (1H, m), 2.04-2.82 (6H, m), 2.98-3.03 (1H, m), 3.10-3.30 (1H, m), 3.65-3.85 (1H, m), 4.04 (1H, d, J=8.3 Hz), 4.02-4.25 (1H, m), 7.13-7.43 (10H, m) MASS (APCI+): 418 (M+H)

Preparation 14

To a solution of tert-butyl (4R,7S,8aS)-4-benzhydryl-7-cyanohexahydropyrrolo[1,2-a]pyrazine-2 (1H)-carboxylate (94 mg) and tetrabutylammonium hydrogensulfate (122 mg) in dichloromethane (1 ml) were added 30% hydrogen peroxide (0.41 ml) and 30% sodium hydroxide aqueous solution (0.41 ml). After stirring at room temperature overnight, the mixture was extracted with dichloromethane (×4). The combined extracts were washed with brine, dried over magnesium sulfate, and evaporated under reduced pressure. The residue was purified with preparative TLC (ethyl acetate) to give two fractions. The upper fraction gave the starting material as an oil (24.1 mg). The lower fraction gave tert-butyl (4R, 7S, 8aS)-4-benzhydryl-7-carbamoylhexahydropyrrolo[1,2-a]pyrazine-2(1H)-carboxylate (39.4 mg) as a white solid.

NMR (CDCl₃, δ): 1.20-1.70 (1H, m), 1.39 (9H, s), 2.04-2.29 (3H, m), 2.40-2.70 (3H, m), 3.08 (1H, d, J=9.7 Hz), 3.00-3.20 (1H, m), 3.85-3.95 (1H, m), 4.00-4.30 (1H, m), 4.16 (1H, d, J=7.5 Hz), 4.88 (1H, brs), 6.24 (1H, brs), 7.19-7.35 (10H, m) MASS (APCI+): 435 (M+H)

Preparation 15

The following compound was obtained according to a similar manner to that of Example 15.

### (4R,8aS)-7-Amino-4-benzhydrylhexahydropyrrolo[1,2-a]pyrazine-2-carboxylic acid tert-butyl ester

MASS (ES+): 466.4 (M+1), 488.4 (M+Na)⁺

Preparation 16

To a solution of ethyl 1-hydroxymethyl-1-cyclopropanecarboxylate (134 mg) in dichloromethane (1.5 ml) were added imidazole (82.3 mg), and tert-butyldimethylsilyl chloride (154 mg) at room temperature. After being stirred at the same temperature for20 hours, the solution was partitioned between ethyl acetate and water, while the aqueous layer was adjusted at pH 3 with diluted hydrochloric acid. The organic layer was washed with brine, dried over magnesium sulfate, and concentrated under reduced pressure. The resulting residue was purified by column chromatography on silica gel (6 g) using a mixed solvent of hexane and ethyl acetate (5:1). The fractions containing the objective compound were collected and evaporated under reduced pressure to give ethyl 1-[(tert-butyldimethylsilyloxy)methyl)-1-cyclopropanecarboxylate (260 mg) as colorless oil.

IR (Neat) : 2952, 2860, 1726, 1466, 1255, 1171, 1097 cm⁻¹ NMR (CDCl₃, δ): −0.07-0.11 (6H, m), 0.81-0.90 (l1H, m), 1.02-1.11 (2H, m), 1.20 (3H, t, J=7.1 Hz), 3.79 (2H, s), 4.08 (2H, q, J=7.1 Hz) MASS (API-ES): 281 (M+Na)⁺

Preparation 17

To a solution of ethyl 1-[(tert-butyldimethylsilyloxy)methyl]-1-cyclopropane carboxylate (250 mg) in ethanol (1.5 ml) was added 1N sodium hydroxide (0.97 ml) under ice-cooling. After being stirred at room temperature for 20 hours, the reaction mixture was adjusted at pH 4 with diluted hydrochloric acid, and the whole was concentrated under reduced pressure. The residue was partitioned between ethyl acetate and diluted hydrochloric acid. The organic layer was separated, dried over magnesium sulfate, and concentrated under reduced pressure. The resulting residue was purified by column chromatography on silica gel (6 g) using a mixed solvent of dichloromethane and methanol (20:1). The fractions containing the objective compound were collected and evaporated under reduced pressure to give 1-(tert-butyldimethylsilyloxy) methyl)-1-cyclopropanecarboxylic acid (85 mg) as colorless oil.

IR (Neat): 2952, 2860, 1693, 1248, 1099 cm⁻¹ NMR (CDCl₃, δ): −0.02-0.12 (6H, m), 0.85-1.40 (13H, m), 3.80 (2H, s) MASS (API-ES): 253 (M+Na)⁺

Preparation 18

To a solution of (4R,9aR)-8-acetyl-4-benzhydryl-2-(2-methoxybenzyl)octahydro-2H-pyrazino[1,2-a]pyrazine (5.9 g) in dichloroethane (60 ml) was added 1-chloroethyl chloroformate (2.3 ml) at room temperature, and the reaction mixture was heated at 70° C. for 30 minutes with stirring. After removal of solvent by evaporation, to the resulting residue was added methanol (45 ml), and the solution was refluxed for 40 minutes. After being concentrated, the residue was triturated with diisopropyl ether. The resulting precipitate was collected by filtration and dried under reduced pressure for 5 hours at 40° C. to give (4R, 9aR)-8-acetyl-4-benzhydryloctahydro-2H-pyrazino[1,2-a]pyrazine dihydrochloride (3.1 g) as colorless foam.

NMR (DMSO-d₆, δ): 1.90-2.00 (3H, m), 2.20-4.70 (13H, m), 7.10-7.50 (10H, m), 9.65 (2H, br) MASS (APCI): 350 (M+H)⁺ (free)

Preparation 19

Under nitrogen atmosphere, to a solution of (2S)-2-ethoxycarbonylpiperazine-1,4-dicarboxylic acid 4-benzyl ester 1-tert-butyl ester (9.35 g) was added portionwise lithium borohydride (1.82 g), and the reaction mixture was stirred for 90 minutes. After methanol (2.32 ml) was added dropwise to the solution under ice-cooling, the mixture was stirred at room temperature for 17 hours. 1N Hydrochloric acid (80 ml) was added dropwise under ice-cooling, and ethyl acetate (100 ml) and sodium chloride (6 g) was added to it. The organic layer was washed with brine, dried over magnesium sulfate, and evaporated under reduced pressure to give colorless oil. The oil was purified by column chromatography on silica gel (90 g) using a mixed solvent of hexane and ethyl acetate (3:2). The fractions containing the objective compound were collected and evaporated under reduced pressure to give (2S)-2-(hydroxymethyl)piperazine-1,4-dicarboxylic acid 4-benzyl ester 1-tert-butyl ester (8.40 g) as a colorless oil.

NMR (CDCl₃, δ): 1.46 (9H, s), 2.40-4.30 (10 H, m), 5.10-5.30 (2H, m), 7.30-7.50 (5H, m) MASS (API-ES): 373 (M+Na)⁺

Preparation 20

Under nitrogen atmosphere, to a solution of oxalyl chloride (1.64 ml) in dichloromethane (34 ml) under −65° C., was added dropwise a solution of dimethyl sulfoxide (2.0 ml) in dichloromethane (15 ml) and stirred for 10 minutes at the same temperature. A solution of (2S)-2-(hydroxymethyl)piperazine-1,4-dicarboxylic acid 4-benzyl ester 1-tert-butyl ester (3.29 g) in dichloromethane (24 ml) was dropped into the above solution over 5 minutes under −65° C. The reaction mixture was stirred at the same temperature for 15 minutes, then stirred at −45° C. for 90 minutes. Triethylamine (7.85 ml) was added to the solution under −40° C., and the mixture was stirred at 0° C. for 20 minutes. The mixture was poured into saturated aqueous ammonium chloride (100 ml). The organic layer was washed with brine, dried over magnesium sulfate, and evaporated to give (2R)-2-formylpiperazine-1,4-dicarboxylic acid 4-benzyl ester 1-tert-butyl ester (3.33 g) as a colorless syrup.

NMR (CDCl₃, δ): 1.40-1.70 (9H, m), 2.85-3.30 (3H, m), 3.70-4.80 (4H, m), 5.05-5.30 (2H, m), 7.30-7.40 (5H, m), 9.58 (1H, s) MASS (API-ES): 371 (M+Na)⁺

Preparation 21

Under nitrogen atmosphere, to a solution of (2R)-2-formylpiperazine-1,4-dicarboxylic acid 4-benzyl ester 1-tert-butyl ester (2.64 g) and 3-(2-methoxybenzylamino)-1,1-diphenylpropan-2-one (3.66 g) in dichloromethane (30 ml) was added acetic acid (0.607 ml) and sodium tritacetoxyborohydride (4.82 g) under ice-cooling, and then it was stirred at room temperature for 3 hours. The reaction mixture was poured into aqueous sodium hydrogen carbonate (100 ml) and extracted with dichloromethane. The organic layer was washed with brine, dried over sodium sulfate, and evaporated under reduced pressure. The resulting residue was purified by column chromatography on silica gel (82 g) using a mixed solvent of hexane and ethyl acetate (3:1). The fractions containing the objective compound were collected and evaporated under reduced pressure to give (2S)-2-[[N-(2-methoxybenzyl)-N-(2-oxo-3,3-diphenylpropyl)amino)methyl]piperazine-1,4-dicarboxylic acid 4-benzyl ester 1-tert-butyl ester (3.24 g) as a syrup.

NMR (CDCl₃, δ): 1.40-1.65 (9H, m), 2.65-5.40 (19H, m), 6.70-7.40 (19H, m) MASS (APCI):. 678 (M+H)⁺

Preparation 22

The following compound was obtained according to a similar manner to that of Preparation 4.

### (R)-3-(N-Methoxy-N-methylcarbamoyl)thiomorpholine-4-carboxylic acid tert-butyl ester

IR (neat): 1695, 1676, 1454, 1394, 1371, 1317, 1163 cm⁻⁴ NMR (CDCl₃, δ): 1.46 (9H, s), 2.50-2.86 (2H, m), 2.93 (1H, dd, J=14.2, 4.9 Hz), 3.05 (1H, dd, J=4.2, 14.2 Hz), 3.22 (3H, S), 3.77 (3H, s), 3.77 (1H, brs), 4.18 (1H, brs), 5.25 (1H, brs) MASS (APCI): 190.8 (M-Boc)⁺

Preparation 23

The following compound was obtained according to a similar manner to the first step of Preparation 5.

### (R)-3-Formylthiomorpholine-4-carboxylic acid tert-butyl ester

IR (neat): 1693 cm⁻¹ MASS (ES−): 230.2 (M−H)⁺

Preparation 24

The following compound was obtained according to a similar manner to that of Preparation 3.

### (R)-3-[(2-Methoxybenzyl)aiming]methyl]thiomorpholine-4-carboxylic acid tert-butyl ester

IR (neat): 1691, 1460, 1412, 1367, 1248, 1163 cm⁻¹ NMR (CDCl₃, δ): 1.46 (9H, s), 2.35 (1H, brd, J=13.0 Hz), 2.54-2.68 (1H, m), 2.71 (1H, dd, J=3.0, 13.0 Hz), 2.85-3.15 (4H, m), 3.81 (2H, s), 3.83 (3H, s), 4.24 (1H, brs), 4.48 (1H, brs), 6.80-7.32 (4H, m) MASS (ES+): 353.2 (M+H)⁺, 375.3 (M+Na)⁺

Preparation 25

The following compound was obtained according to a similar manner for to that of Preparation 2.

### (R)-3-[[N-(2-Methoxybenzyl)-N-(2-oxo-3,3-diphenylpropyl)amino]methyl]thiomorpholine-4-carboxylic acid tert-butyl ester

IR (neat): 1723, 1685, 1240, 1159 cm⁻¹ NMR (CDCl₃, δ): 1.41 (9H, s), 2.27 (1H, brd, J=12.9 Hz), 2.55 (1H, dd, J=2.9, 12.3 Hz), 2.50-2.96 (5H, m), 3.19 (1H, brs), 3.40 (1H, d, J=17.2 Hz), 3.53 (1H, d, J=17.2 Hz), 3.66 (1H, J=13.7 Hz), 3.74 (3H, s), 3.90 (1H, d, J=13.7 Hz), 4.12 (1H, brs), 4.36 (1H, brs), 6.74-7.38 (14H, m) MASS (APCI): 561 (M+H)⁺

Preparation 26

The following compound was obtained according to a similar manner to that of Preparation 18.

### (6R,9aR)-6-Benzhydryloctahydropyrazino[2,1-c][1,4]thiazine dihydrochloride

MASS (APCI): 324 (M+H)⁺ (free)

Preparation 27

Sodium triacetoxyborohydride (127 mg) was added portionwise to a mixture of 2-benzhydrylpiperazine dihydrochloride (97.6 mg), N,N-diisopropylethylamine (0.104 ml) and 2-methoxy-5-[5-(trifluoromethyl)tetrazol-1-yl]benzaldehyde (61.2 mg) in a mixture of dichloromethane (5 ml) and acetic acid (1 drop) at 0° C. and the whole was stirred at 5° C. —room temperature overnight. The mixture was partitioned between ethyl acetate and 2N sodium hydroxide. The organic layer was separated, washed with brine, dried over sodium sulfate and evaporated under reduced pressure. The resulting residue was purified by column chromatography on silica gel using a mixed solvent of dichloromethane and methanol (70:1). The fractions containing the objective compound were collected, evaporated under reduced pressure and treated with 4N hydrogen chloride in ethyl acetate solution to give 3-benzhydryl-1-[2-methoxy-5-[5-(trifluoromethyl)-1H-tetrazol-1-yl]benzyl]piperazine dihydrochloride (74 mg) as a colorless powder.

NMR (DMSO-d₆, δ): 2.60-4.81 (14H, m), 7.17-7.50 (11H, m), 7.22-7.75 (2H, m) MASS (APCI): 509 (M+H)⁺ (free)

Preparation 28

Sodium triacetoxyborohydride (146 mg) was added portionwise to a mixture of 37% aqueous formaldehyde (30 mg) and 3-benzhydryl-1-(2-methoxy-5-[5-(trifluoromethyl)-1H-tetrazol-1-yl)benzyl]piperazine dihydrochloride in a mixture of dichloromethane (4 ml) and methanol (2 drops) at 0° C. and the whole was stirred at 5° C. —room temperature overnight. The mixture was partitioned between ethyl acetate and 2N sodium hydroxide. The organic layer was separated, washed with brine, dried over sodium sulfate and evaporated under reduced pressure. The resulting residue was purified by column chromatography on silica gel using a mixed solvent of dichloromethane and methanol (60:1). The fractions containing the objective compound were collected and evaporated under reduced pressure and treated with 4N hydrogen chloride in ethyl acetate solution to give 2-benzhydryl-4-[2-methoxy-5-[5-(trifluoromethyl)-1H-tetrazol-1-yl]benzyl]-1-methylpiperazine dihydrochloride (32.9 mg) as a colorless powder.

NMR (DMSO-d₆, δ): 2.28-4.73 (16H, m), 7.15-7.40 (9H, m), 7.55 (2H, m), 7.71 (2H, m) MASS (APCI): 523 (M+H)⁺ (free)

Preparation 29

Lithium aluminum hydride (198 mg) was added by small portions to an ice-cooled solution of 1,4-dibenzyl-3-benzhydryl-2,5-piperazinedione (800 mg) in tetrahydrofuran (8 ml) under nitrogen atmosphere, and the mixture was stirred under reflux for 5 hours. After being cooled with ice, 2N sodium hydroxide (1 ml) was added to the mixture under nitrogen atmosphere. The resulting precipitates were filtered off and washed with tetrahydrofuran, and the filtrate and the washings were combined and evaporated under reduced pressure to give a crude oil. The oil was purified by column chromatography on silsica gel using a mixed solvent of hexane and ethyl acetate (9:1). The fractions containing the objective compound were collected, evaporated under reduced pressure and treated with 4N hydrogen chloride in ethyl acetate solution to give 1,4-dibenzyl-2-benzhydrylpiperazine dihydrochloride (846 mg) as a colorless powder.

NMR (DMSO-d₆, δ): 2.30-6.50 (12H, m), 7.03-7.98 (20H, m) MASS (APCI): 433 (M+H)⁺ (free)

Preparation 30

(6R,9aR)-6-Benzhydryl-8-(tert-butoxycarbonyl)-octahydropyrazino[2,1-c][1,4]oxazine was treated with 4N hydrogen chloride in 1,4-dioxane to give (6R,9aR)-octahydro-6-benzhydrylpyrazino[2,1-c][1,4]oxazine dihydrochloride as a yellowish powder. (6R,9aR)-6-Benzhydryl-8-[2-methoxy-5-[5-(trifluoromethyl)-1H-tetrazol-1-yl]benzyl]octahydropyrazino[2,1-c][1,4]oxazine dihydrochloride was obtained from (6R,9aR)-6-benzhydrylhexahydropyrazino[2,1-c][1,4]oxazine dihydrochloride according to a similar manner to that of Example 2.

NMR (DMSO-d₆, δ): 2.07-2.60 (3H, m), 2.75-4.54 (17H, m), 7.18-7.78 (13H, m) MASS (APCI): 565 (M+H)⁺ (free)

Preparation 31

Acetyl chloride (3 drops) was added to a mixture of (6R,9aS)-4-benzhydryl-2-(2-methoxybenzyl)octahydropyrazino[1,2-a]pyrazine trihydrochloride (20 mg) and N,N-diisopropylethylamine (6 drops) in dichloromethane (1 ml) under ice-cooling. After being stirred at the same temperature for 2 hours, the mixture was poured into ice-water and extracted with ethyl acetate. The extract was washed with brine, dried over sodium sulfate and evaporated under reduced pressure to give a crude oil. The oil was purified by column chromatography on silica gel using a mixed solvent of dichloromethane and methanol (50:1) as an eluent. The fractions containing the objective compound were collected and evaporated under reduced pressure and the resulting residue was treated with 4N hydrogen chloride in ethyl acetate to give 1-[(6R,9aR)-6-benzhydryl-8-(2-methoxybenzyl)octahydropyrazino[1,2-a]pyrazin-2-yl]ethanone dihydrochloride (9.8 mg) as a colorless powder.

NMR (DMSO-d₆, δ): 1.90-4.60 (21H, m), 6.95-7.39 (14H, m) MASS (APCI): 470 (M+H)⁺ (free)

### EXAMPLE 1

The following compound was obtained according to a similar manner to that of Preparation 28.

### 2-(2-Benzhydryl-4-(2-methoxy-5-(5-(trifluoromethyl)-1H-tetrazol-1-yl)benzyl]-1-piperazinyl]acetic acid

MASS (APCI): 567 (M+H)⁺

Dihydrochloride of the above compound IR (KBr, FT-IR): 1615, 1440, 1320, 1265, 1235 cm⁻¹ NMR (DMSO-d₆, δ): 2.70-5.15 (12H, m), 3.84 (3H, s), 7.10-8.10 (13H, m), 10.36 (1H, br s) MASS (APCI): 567 (M+H)⁺ (free)

### EXAMPLE 2

Thionyl chloride (0.5 ml) was added dropwise to an ice-cooled methanol (3 ml) over 15 minutes. The mixture was stirred for 15 minutes and thereto 2-[2-benzhydryl-4-[2-methoxy-5-[5-(trifluoromethyl)-1H-tetrazol-1-yl]benzyl]-1-piperazinyl]acetic acid (93 mg) was added. The whole mixture was stirred at room temperature overnight and evaporated under reduced pressure. The syrup was partitioned between aqueous sodium hydrogen carbonate and dichloromethane. The organic layer was separated, dried over magnesium sulfate and evaporated under reduced pressure. The syrup was purified by column chromatography on silica gel using a mixed solvent of dichloromethane and methanol (20:1). The fractions containing the objective compound were collected and evaporated under reduced pressure to give a syrup. The syrup was treated with 4N hydrogen chloride in ethyl acetate (1 ml) to give methyl [2-benzhydryl-4-[2-methoxy-5-[5-(trifluoromethyl)-1H-tetrazol-1-yl]benzyl]-1-piperazinyl]acetate dihydrochloride (44 mg).

IR (KBr, FT-IR): 1735, 1500, 1440, 1320, 1265 cm⁻¹ NMR (DMSO-d₆, δ): 1.93-4.65 (10H, m), 3.32 (3H, s), 3.46 (3H, s), 3.83 (2H, s), 6.98-8.25 (13H, m) MS (APCI): 581 (M+H)⁺ (free)

### EXAMPLE 3

The following compound was obtained according to a similar manner to that of Preparation 31.

### 1-Acetyl-2-benzhydryl-4-[2-methoxy-5-(5-(trifluoromethyl)-1H-tetrazol-1-yl]benzyl]piperazine dihydrochloride

IR (KBr, FT-IR): 1655, 1635, 1440, 1415, 1320, 1265 cm⁻¹ NMR (DMSO-₆, δ): 1.81 (3H, s), 2.65-5.60 (10H, m), 3.49 (3H, s), 7.05-8.15 (13H, m) MASS (APCI): 551 (M+H)⁺ (free)

### EXAMPLE 4

The following compounds were obtained according to a similar manner to that of Preparation 27 from (4R,8aS)-4-benzhydryloctahydropyrrolo[1,2-a]pyrazine dihydrochloride.

### Benzyl [(4R,7S,8aS)-4-benzhydryl-2-[2-methoxy-5-(5-(trifluoromethyl)-1H-tetrazol-1-yl]benzyl]octahydropyrrolo[1,2-a]pyrazin-7-yl] carbamate

MASS (APCI): 698 (M+H)⁺

Dihydrochloride of the above compound

IR (KBr): 2900-2500, 1716, 1504 cm⁻¹ NMR (CDCl₃, δ): 1.80-2.60 (14H, m), 2.86 (1H, d, J=10.2 Hz), 3.20-3.32 (1H, m), 3.44 (1H, d, J=15.1 Hz), 3.55 (1H, d, J=15.1 Hz), 3.80 (3H, s), 3.95 (1H, d, J=8.5 Hz), 4.95 (1H, d, J=8.6 Hz), 5.04 (2H, s), 6.92 (1H, d, J=8.8 Hz, 7.06-7.34 (11H, m), 7.42 (1H, d, J=2.6 Hz) MASS (APCI): 698 (M+H)⁺ (free)

### EXAMPLE 5

Sodium triacetoxyborohydride (163 mg) was added to a mixture of bis(acetic acid) salt of (7R,8aS)-4-benzhydryl-7-[(tert-butyldimethylsilyl)oxy)octahydropyrrolo[1,2-a]pyrazine (0.38 g) and 2-methoxy-5-[5-(trifluoromethyl)-1H-tetrazol-1-yl]benzaldehyde (210 mg) in dichloromethane, and the whole was stirred for 3 hours at room temperature. The mixture was washed with aqueous sodium hydrogen carbonate, dried over magnesium sulfate and concentrated under reduced pressure. The syrup was purified by column chromatography on silica gel using a mixed solvent of hexane and ethyl acetate (4:1). The later eluting fractions were collected and evaporated under reduced pressure to give colorless oil of (4R, 7R, 8aS)-4-benzhydryl-7-[(tert-butyldimethylsilyl)oxy]-2-[2-methoxy-5-[5-(trifluoromethyl)-1H-tetrazol-1-yl]benzyl]octahydropyrrolo[1,2-a]pyrazine (0.18 g).

NMR (CDCl₃, δ): −0.20 (3H, s), −0.11 (3H, m), 0.75 (9H, m), 1.58-1.74 (4H, m), 2.18 (1H, dd, J=4.7 and 9.6 Hz), 2.26 (1H, dd, J=3.3 and 11.3 Hz), 2.31 (1H, d, J=11.3 Hz), 2.69 (1H, dd, J=3.0 and 10.6 Hz), 2.96 (1H, dd, J=6.7 and 9.5 Hz), 3.25 (1H, d, J=14.8 Hz), 3.30-3.50 (1H, m), 3.69 (1H, d, J=10.6 Hz), 3.87 (3H, s), 4.20-4.25 (1H, m), 4.66 (1H, d, J=10.8 Hz), 6.94-7.40 (12H, m), 7.54 (1H, d, J=2.6 Hz) MASS (APCI-ES): 679 (M+H)⁺

The earlier eluting fractions were collected and evaporated under reduced pressure to give colorless oil of (4S,7R,8as)-4-benzhydryl-7-[(tert-butyldimethylsilyl)oxy]-2-[2-methoxy-5-[5-(trifluoromethyl)-1H-tetrazol-1-yl]benzyl)octahydropyrrolo[1,2-a]pyrazine (0.15 g).

NMR (CDCl₃, δ): −0.20 (3H, s), −0.11 (3H, m), 0.75 (9H, m), 1.56-1.95 (6H, m), 2.47 (1H, d, J=11.2Hz), 2.64-2.92 (2H, m), 3.36-3.60 (3H, m), 2.78 (3H, s), 3.92 (1H, d, J=11.1 Hz), 4.07-4.17 (1H, m), 6.92 (1H, d, J=8.8 Hz), 7.05-7.45 (12H, m) MASS (APCI-ES): 679 (M+H)⁺ (free)

### EXAMPLE 6

The following compounds were obtained according to a similar manner to that of Preparation 30.

### (1) N-[(4R,7S,8aS)-4-Benzhydryl-2-[2-methoxy-5-[5-(trifluoromethyl)-1H-tetrazol-1-yl]benzyl]octahydropyrrolo[1,2-a]pyrazin-7-yl]acetamide dihydrochloride

IR (KBr): 3400, 1648, 1504 cm⁻¹ NMR (DMSO-d₆, δ): 1.48 (1H, br s), 1.76 (3H, s), 2.30-5.00 (12H, m), 3.76 (3H, s), 7.16-7.77 (13H, m), 8.21 (1H, br s) MASS (APCI): 606 (M+H)⁺ (free)

### (2) (4R,8aS)-4-Benzhydryl-2-[2-methoxy-5-[5-(trifluoromethyl)-1H-tetrazol-1-yl]benzyl]hexahydropyrrolo[1,2-a]pyrazin-7(6H)-one dihydrochloride

NMR (DMSO₆, δ): 2.15-4.30 (17H, m), 7.18-8.08 (13H, m), 10.37 (1H, m) MASS (APCI): 563 (M+H)⁺ (free)

### (3) (4R,8aS)-4-Benzhydryl-7,7-difluoro-2-[2-methoxy-5-[5-(trifluoromethyl)-1H-tetrazol-1-yl]benzyl]octahydropyrrolo[1,2-a]pyrazine dihydrochloride

NMR (DMSO-₆, δ): 2.15-4.30 (17H, m), 7.20-7.85 (13H, m), 10.5 (1H, br) MASS (APCI): 585 (M+H)⁺ (free)

### (4) N-[(4R,7R,8aS)-4-Benzhydryl-2-[2-methoxy-5-[5-(trifluoromethyl)-1H-tetrazol-1-yl]benzyl]octahydropyrrolo[1,2-a]pyrazin-7-yl]acetamide dihydrochloride

NMR (DMSO-d₆, δ): 1.80-4.55 (23H, m), 7.21-8.14 (13H, m) MASS (APCI): 606 (M+H)⁺ (free)

### (5) (4R,7S,8aS)-4-Benzhydryl-7-cyano-2-[2-methoxy-5-[5-(trifluoromethyl)-1H-tetrazol-1-yl]benzyl]octahydropyrrolo[1,2-a]pyrazine dihydrochloride

IR (KBr): 3435, 1506 cm⁻¹ NMR (DMSO-d₆, δ): 2.20-4.30 (14H, m), 3.78 (3H, s), 7.21-7.84 (13H, m) MASS (APCI−): 327 (M−H)

### (6) (4R,7S,8aS)-4-Benzhydryl-7-carbamoyl-2-[2-methoxy-5-[5-(trifluoromethyl)-1H-tetrazol-1-yl]benzyl]octahydropyrrolo[1,2-a]pyrazine dihydrochloride

[α]_{D}²⁵: −17.684(C, 0.095, MeOH) IR (KBr): 1684 cm⁻¹ NMR (DMSO-d₆, δ): 2.20-4.80 (14H, m), 3.77 (3H, s), 7.06-7.74 (13H, m) MASS (APCI): 592 (M+H)

### (7) (4R,8aS)-N-[4-Benzhydryl-2-[2-methoxy-5-(5-trifluoromethyl-tetrazol-1-yl]benzyl]octahydropyrrolo[1,2-a]pyrazin-7-yl]-2-hydroxyacetamide dihydrochloride

mp 155-159° C. [α]_{D}²⁵: −18.852 (c=0.061, MeOH) IR (KBr): 3396, 1645, 1535, 1514, 1200, 1165 cm⁻¹ NMR (CDCl₃, δ): 1.40-5.50 (19H, m), 6.80-8.10 (13H, m) MASS (ES+): 622.3 (M+H)⁺, 644.2 (M+Na)⁺

### EXAMPLE 7

Acetic anhydride (18 mg) was added dropwise to an ice-cooled solution of (4R,7S,8aS)-4-benzhydryl-2-[2-methoxy-5-[5-(trifluoromethyl)-1H-tetrazol-1-yl]benzyl]octahydropyrrolo[1,2-a]pyrazin-7-ol dihydrochloride (58.3 mg), and pyridine (36.2 mg) in dichloromethane (1 ml). After being stirred at the same temperature for 2 hours, triethylamine (27.8 mg) was added to the mixture and the whole was stirred at room temperature overnight. The mixture was poured into ice-water and extracted with dichloromethane. The extract was washed with brine, dried over sodium sulfate and evaporated under reduced pressure to give a crude oil. The resulting residue was purified by preparative silica gel column chromatography with a mixture of hexane and ethyl acetate (1:2) as an eluent. The obtained oil was treated with 4N hydrogen chloride in ethyl acetate solution to give (4R,7S,8aS)-4-benzhydryl-2-[2-methoxy-5-[5-(trifluoromethyl)-1H-tetrazol-1-yl]benzyl]octahydropyrrolo[1,2-a]pyrazin-7-yl acetate dihydrochloride (57.6 mg).

NMR (DMSO-d₆, δ): 1. 40-4.87 (22H, m), 7.21-7.77 (13H, m) MASS (APCI): 606 (M+H)⁺ (free)

Preparation 32

To a solution of (2S)-2-[[N-(2-methoxybenzyl)-N-(2-oxo-3,3-diphenylpropyl)amino]methyl]piperazine-1,4-dicarboxylic acid 4-N-benzyl ester 1-N-tert-butyl ester (3.15 g) in ethyl acetate (15 ml) was added a solution of 4N hydrogen chloride in ethyl acetate (29.6 ml) under ice-cooling. After stirring at the same temperature for 3 hours, the reaction mixture was evaporated under reduced pressure. To the solution of the residue in dichloromethane (30 ml) was added portionwise sodium triacetoxyborohydride (2.95 g) under ice-cooling, and then it was stirred at the same temperature for 20 hours. The mixture was poured into aqueous sodium hydrogen carbonate and extracted with dichloromethane. The organic layer was washed with brine, dried over sodium sulfate, evaporated under reduced pressure. The resulting residue was purified by column chromatography on silica gel (5.2 g) using a mixed solvent of hexane and ethyl acetate (2:1). The fractions containing the objective compound were collected and evaporated under reduced pressure to give (4S,9aS)-8-(benzyloxycarbonyl)-4-benzhydryl-2-(2-methoxybenzyl)octahydro-2H-pyrazino[1,2-a]pyrazine (2.0 g) as a syrup.

NMR (CDCl₃, δ): 3.68 (3H, s), 1.75-4.25 (15H, m), 5.08 (2H, s), 6.70-6.90 (2H, m), 7.10-7.40 (17H, m) MASS (APCI): 562 (M+H)⁺

### EXAMPLE 8

The following compound was obtained according to a similar manner to that of Preparation 8 from (4R,8S,8aR)-4-benzhydryl-2-[2-methoxy-5-[5-(trifluoromethyl)-1H-tetrazol-1-yl]benzyl]octahydropyrrolo[1,2-a]pyrazin-8-ol.

### (4R,8aR)-4-Benzhydryl-2-[2-methoxy-5-[5-(trifluoromethyl)-1H-tetrazol-1-yl]benzyl]hexahydropyrrolo[1,2-a]pyrazin-8(2H)-one dihydrochloride

NMR (DMSO-d₆, δ): 1.98-4.24 (18H, m), 7.21-7.80 (13H, m) ASS (APCI): 563 (M+1) (free)

### EXAMPLE 9

The following compound was obtained according to a similar manner to that of Preparation 6 from (4R,8S,8aR)-4-benzhydryl-2-[2-methoxy-5-[5-(trifluoromethyl)-1H-tetrazol-1-yl]benzyl]octahydropyrrolo[1,2-a]pyrazin-8-ol.

### (4R,8R,8aR)-8-Azido-4-benzhydryl-2-[2-methoxy-5-[5-(trifluoromethyl)-1H-tetrazol-1-yl]benzyl]octahydropyrrolo[1,2-a]pyrazine

MASS (APCI): 590 (M+1)

### EXAMPLE 10

The following compound was obtained according to a similar manner to that of Preparation 7 from (4R,8R,8aR)-4-benzhydryl-2-[2-methoxy-5-[5-(trifluoromethyl)-1H-tetrazol-1-yl]benzyl]octahydropyrrolo[1,2-a]pyrazin-8-amine.

### N-[(4R, 8R,8aR)-4-Benzhydryl-2-[2-methoxy-5-[5-(trifluoromethyl)-1H-tetrazol-1-yl]benzyl]octahydropyrrolo[1,2-a]pyrazin-8-yl]acetamide dihydrochloride

NMR (DMSO-d₆, δ): 1.23-4.30 (21H, m), 7.21-7.56 (13H, m) MASS (APCI): 606 (M+1)

### EXAMPLE 11

The following compound was obtained according to a similar manner to that of Preparation 10 from (4R,8S,8aR)-4-benzhydryl-2-[2-methoxy-5-[5-(trifluoromethyl)-1H-tetrazol-1-yl]benzyl]octahydropyrrolo[1,2-a]pyrazin-8-ol.

### (4R,8R,8aR)-4-Benzhydryl-2-[2-methoxy-5-[5-(trifluoromethyl)-1H-tetrazol-1-yl]benzyl]octahydropyrrolo[1,2-a]pyrazin-8-yl acetate NMR (CDCl₃, δ): 1.91-2.23 (5H, m), 2.03 (3H, s), 2.43 (2H, br), 2.63-2.89 (2H, m), 3.24 (1H, br), 3.42-3.64 (2H, d×2, J=15 Hz), 3.78 (3H, s), 4.09 (1H, m), 5.18 (1H, m), 6.90-7.42 (13H, m)

MASS (APCI) : 607 (N+1)

### EXAMPLE 12

The following compound was obtained according to a similar manner to that of Preparation 6 from (4R,8R,8aR)-4-benzhydryl-2-[2-methoxy-5-[5-(trifluoramethyl)-1H-tetrazol-1-yl]benzyl]octahydropyrrolo[1,2-a]pyrazin-8-ol.

### (4R,8S,8aR)-8-Azido-4-benzhydryl-2-[2-methoxy-5-[5-(trifluoromethyl)-1H-tetrazol-1-yl]benzyl]octahydropyrrolo[1,2-a]pyrazine

MASS (APCI): 590 (M+1) (free)

### EXAMPLE 13

The following compound was obtained according to a similar manner to that of Preparation 7 from (4R,8S,8aR)-4-benzhydryl-2-[2-methoxy-5-[5-(trifluoromethyl)-1H-tetrazol-1-yl]benzyl]octahydropyrrolo[1,2-a]pyrazin-8-amine.

### N-[(4R,8S,8aR)-4-Benzhydryl-2-[2-methoxy-5-[5-(trifluoromethyl)-1H-tetrazol-1-yl]benzyl]octahydropyrrolo[1,2-a]pyrazin-8-yl]acetamide dihydrochloride

NMR (DMSO-d₆, δ): 1.14-4.77 (23H, m), 6.82-8.16 (13H, m) MASS (APCI): 606 (M+1)

### EXAMPLE 14

The following compounds were obtained according to a similar manner to that of Preparation 31.

### (1) (4R, 9aR)-8-Acetoxyacetyl-4-benzhydryl-2-[2-methoxy-5-[5-(trifluoromethyl)-1H-tetrazol-1-yl]benzyl]octahydro-2H-pyrazino[1,2-a]pyrazine dihydrochloride

mp: 138-153° C. [α]_{D}²⁶−39.70 (C, 0.11, MeOH) IR (KBr): 1743, 1676, 1653 cm⁻¹ NMR (DMSO-d₆, δ): 2.02 and 2.05 (total 3H, s), 2.20-4.80 (17H, m), 3.80 and 3.85 (total 3H, s), 7.18-7.80 (13H, m) MASS (APCI+): 664.1 (M+H)⁺ (free)

### (2) (4R, 9aR)-8-(2-Acetoxy-2-methylpropionyl)-4-benzhydryl-2-[2-methoxy-5-[5-(trifluoromethyl)-1H-tetrazol-1-yl]benzyl]octahydro-2H-pyrazino[1,2-a]pyrazine dihydrochloride

mp: 143-148° C. IR (KBr): 1738, 1647 cm⁻¹ NMR (DMSO-d₆, δ): 1.42 (3H, s), 1.45 (3H, s), 2.00 (3H, s), 2.20-4.40 (15H, m), 3.83 (3H, s), 7.18-7.90 (13H, m) MASS (APCI+): 692.2 (M+H)⁺ (free)

### (3) (4R, 9aR)-4-Benzhydryl-8-cyclohexanecarbonyl-2-[2-methoxy-5-(5-(trifluoromethyl)-1H-tetrazol-1-yl]benzyl]octahydro-2H-pyrazino[1,2-a]pyrazine dihydrochloride

mp: 169-174.5° C. [α]_{D}²⁸: −36.40 (C, 0.125, MeOH) IR (KBr): 1647 cm⁻¹ NMR (DMSO-d₆, δ): 1.00-1.80 (10H, m), 2.20-4.40 (16H, m), 3.80 (3H, s), 7.14-7.81 (13H, m) MASS (APCI+): 674.2 (M+H)⁺ (free)

### (4) (4R, 9aR)-4-Benzhydryl-8-cyclopropanecarbonyl-2-[2-methoxy-5-[5-(trifluoromethyl)-1H-tetrazol-1-yl]benzyl octahydro-2H-pyrazino[1,2-a)pyrazine dihydrochloride

mp: 165-168° C. [α]_{D}^{30.0}: −42.24° (C=0.29, MeOH) IR (KBr) : 3435, 1645, 1504, 1460, 1265, 1201, 1165, 1034 cm⁻¹ NMR (DMSO-d₆, δ): 0.65-0.82 (4H, m), 1.90-4.50 (16H, m), 3.81 (3H, s), 7.10-7.50 (11H, m), 7.70-7.90 (2H, m) MASS (APCI): 632 (M+H)⁺ (free)

### (5) (4R, 9aR)-4-Benzhydryl-2-[2-methoxy-5-[5-(trifluoroethyl)-1H-tetrazol-1-yl]benzyl)-8-(3-pyridylcarbonyl)octahydro-2H-pyrazino[1,2-a]pyrazine trihydrochloride

mp: 197-200° C. [α]_{D}^{30.0}: −42.46° (C=0.325, MeOH) IR (KBr) : 3404, 1649, 1504, 1458, 1269, 1199, 1165 cm⁻¹ NMR (DMSO-d₆, δ): 2.20-5.00 (15H, m), 3.80 (3H, s), 7.10-7.50 (11H, m), 7.70-7.95 (3H, m), 8.32 (1H, s), 8.86 (1H, dd, J=1.3 Hz, J=5.4 Hz), 8.92 (1H, s) MASS (APCI): 668(M)⁺ (free)

### (6) (4R,9aR)-4-Benzhydryl-2-(2-methoxy-5-[5-(trifluoromethyl)-1H-tetrazol-1-yl]benzyl]-8-(trifluoroacetyl)octahydro-2H-pyrazino[1,2-a]pyrazine dihydrochloride

NMR (DMSO-d₆, δ): 2.30-4.25 (20H, m), 7.17-8.14 (13H, m) MASS (APCI): 660 (M+H)⁺ (free)

### (7) (4R, 9aR)-4-Benzhydryl-8-(methoxyacetyl)-2-[2-methoxy-5-[5-(trifluoromethyl)-1H-tetrazol-1-yl]benzyl]octahydro-2H-pyrazino (1,2-a]pyrazine dihydrochloride

NMR (DMSO-d₆, δ): 2.21-4.28 (23H, m), 7.17-8.14 (13H, m), 10.24-10.27 (2H, m) MASS (APCI): 636 (M+H)⁺ (free)

### (8) Methyl 3-(6R, 9aR)-6-benzhydryl-8-[2-methoxy-5-(5-trifluoromethyl-1H-tetrazol-1-yl]benzyl]octahydro-2H-pyrazino[1,2-a]pyrazin-2-yl]-3-oxopropanoate IR (KBr): 1741, 1645 cm⁻¹ MASS (APCI) : 664.07 (M+H)⁺ (free)

Dihydrochloride of the above compound

IR (KBr): 1741, 1651 cm⁻¹ NMR (DMSO-d₆, δ): 2.20-4.40 (23H, m), 7.21-7.90 (13H, m) MASS (APCI+): 664.1 (M+H)⁺ (free)

### EXAMPLE 15

To a solution of (4R,9aS)-4-benzhydryl-2-[2-methoxy-5-[5-(trifluoromethyl)-1H-tetrazol-1-yl]benzyl]-octahydro-2H-pyrazino[1,2-a]pyrazine trihydrochloride (120 mg) in dichloromethane (1.0 ml) were added N,N-diisopropylethylamine (0.186 ml) and acetoxyacetyl chloride (28.8 ml) at 0° C. After stirring at 0° C. for 1 hour, the mixture was quenched with aqueous saturated sodium hydrogen carbonate (15 ml) and extracted with dichloromethane (20 ml×2). The combined organic extracts were washed with brine, dried over magnesium sulfate, and evaporated under reduced pressure to give solid (123 mg). To a solution of the solid in methanol (2 ml) was added potassium carbonate (37 mg). After stirring at room temperature for 1 hour, the mixture was evaporated under reduced pressure. The residue was portioned between brine and dichloromethane. The organic layer was dried over magnesium sulfate and evaporated under reduced pressure. The residue was purified with preparative TLC (methanol/chloroform=1/19) to give an oil. To a solution of the oil in ethyl acetate (1 ml) was added 4N hydrogen chloride in ethyl acetate (0.5 ml) and hexane (20 ml). After stirring for 30 minutes, the precipitate was collected by filtration and dried under reduced pressure at 50° C. for 5 hours to give (4R,9aR)-4-benzhydryl-8-hydroxyacetyl-2-[2-methoxy-5-[5-(trifluoromethyl)-1H-tetrazol-1-yl]benzyl]octahydro-2H-pyrazino[1,2-a]pyrazine dihydrochloride (96.3 mg) as a white solid. mp: 140-159° C. [α]_{D}²⁶: −46.03 (C, 0.105, MeOH) IR (KBr) : 1649, 1508 cm⁻¹ NMR (DMSO-d₆, δ): 2.20-4.50 (17H, m), 3.82 (3H, s), 7.18-7.82 (13H, m) MASS (APCI): 622 (M+H)⁺, 644 (M+Na)⁺

### EXAMPLE 16

The following compounds were obtained according to a similar manner to that of Example 15.

### (1) (4R,9aR)-4-Benzhydryl-8-(3-hydroxypropionyl)-2-[2-methoxy-5-[5-(trifluoromethyl)-1H-tetrazol-1-yl]benzyl]octahydro-2H-pyrazino[1,2-a]pyrazine dihydrochloride

mp: 147-154° C. [α]_{D}²⁷: −34.67 (C, 0.125, MeOH) IR (KBr): 1645 cm⁻¹ NMR (DMSO-d₆, δ): 2.10-4.40 (19H, m), 3.82 (3H, s), 7.18-7.82 (13H, m) MASS (APCI+): 635.9 (M+H)⁺ (free)

### (2) (4R,9aR)-4-Benzhydryl-8-[(2S)-2-hydroxypropionyl)-2-[2-methoxy-5-[5-(trifluoromethyl)-1H-tetrazol-1-yl]benzyl]octahydro-2H-pyrazino[1,2-a]pyrazine dihydrochloride

mp: 150-158° C. [α]_{D}²⁷: −31.33 (C, 0.125, MeOH) IR (KBr): 1647 cm⁻¹ NMR (DMSO-d₆, δ): 1.13 (3H, d, J=6.4 Hz), 3.82 (3H, s), 2.00-4.40 (16H, m), 7.18-7.82 (13H, m) MASS (APCI+): 635.87 (M+H)⁺ (free)

### (3) (4R, 9aR)-4-Benzhydryl-8-(2-hydroxy-2-methylpropionyl)-2-[2-methoxy-5-[5-(trifluoromethyl)-1H-tetrazol-1-yl]benzyl]octahydro-2H-pyrazino[1,2-a]pyrazine dihydrochloride

[α]_{D}²⁷: −35.33 (C, 0.125, MeOH) IR (KBr): 1647 cm⁻¹ NMR (DMSO-d₆, δ): 1.26 (3H, s), 1.28 (3H, s), 2.20-4.40 (15H, m), 3.80 (3H, s), 7.18-7.81 (13H, m) MASS (APCI+): 650.1 (M+H)⁺ (free)

### EXAMPLE 17

To a mixture of (4R,9aS)-4-benzhydryl-2-[2-methoxy-5-(5-(trifluoromethyl)-1H-tetrazol-1-yl]benzyl]octahydro-2H-pyrazino[1,2-a]pyrazine trihydrochloride (80 mg), cyclopentanecarboxylic acid (16.9 g l), 1-hydroxybenzotriazole hydrate (23 mg), and triethylamine (79 μl) in dichloromethane (1 ml) was added 1-[3-(dimethylamino)propyl]-3-ethylcarbodiimide hydrochloride at room temperature. After stirring at room temperature overnight, the mixture was quenched with aqueous saturated sodium hydrogen carbonate and extracted with dichloromethane. The extract was dried over magnesium sulfate and evaporated under reduced pressure. The residue was purified with preparative TLC (methanol/chloroform=1/9) to give an oil. To a solution of the oil in ethyl acetate (1 ml) was added 4N hydrogen chloride in ethyl acetate (0.2 ml) and hexane (20 ml). After stirring for 30 minutes, the precipitate was collected by filtration and dried under reduced pressure at 50° C. for 5 hours to give (4R,9aR)-4-benzhydryl-8-cyclopentanecarbonyl-2-[2-methoxy-5-[5-(trifluoromethyl)-1H-tetrazol-1-yl]benzyl]octahydro-2H-pyrazino[1,2-a]pyrazine dihydrochloride (63.9 mg) as a powder.

mp: 170-178° C., decomp. [α]_{D}²⁷: −37.83 (C, 0.115, MeOH) IR (KBr) 1647 cm⁻¹ NMR (DMSO-d₆, δ): 1.40-1.80 (8H, m), 2.20-4.50 (16H, m), 3.80 and 3.82 (total 3H, s), 7.15-7.82 (13H, m) MASS (APCI+): 660.2 (M+H)⁺ (free)

### EXAMPLE 18

The following compounds were obtained according to a similar manner to that of Example 17.

### (1) (4R,9aR)-4-Benzhydryl-8-cyclobutanecarbonyl-2-[2-methoxy-5-(5-(trifluoramethyl)-1H-tetrazol-1-yl]benzyl]octahydro-2H-pyrazino[1,2-a]pyrazine dihydrochloride

mp: 155-167° C., decomp. [α]_{D}²⁷: −41.40 (C, 0.095, MeOH) IR (KBr): 1647 cm⁻¹ NMR (DMSO-d₆, δ): 1.60-4.40 (22H, m), 3.18 and 3.83 (total 3H, s), 7.18-7.82 (13H, m) MASS (APCI+): 646.1 (M+H)⁺ (free)

### (2) (4R,9aR)-4-Benzhydryl-8-(3-methoxypropionyl)-2-[2-methoxy-5-[5-(trifluoromethyl)-1H-tetrazol-1-yl]benzyl]octahydro-2H-pyrazino[1,2-a]pyrazine dihydrochloride

mp: 132-135° C. [α]_{D}²⁷: −35.17 (C, 0.1, MeOH) IR (KBr): 1649 cm⁻¹ NMR (DMSO-₆, δ): 2.20-4.40 (19H, m), 3.19 (3H, s), 3.80 and 3.83 (total 3H, s), 7.15-7.81 (13H, m) MASS (APCI+): 650.1 (M+H)⁺ (free)

### (3) (4R,9aR)-4-Benzhydryl-8-(3,3,3-trifluoropropionyl)-2-[2-methoxy-5-[5-(trifluoromethyl)-1H-tetrazol-1-yl]benzyl]octahydro-2H-pyrazino[1,2-a]pyrazine dihydrochloride

mp: 155-160° C. [α]_{D}²⁶: −31.48 (C, 0.135, MeOH) IR (KBr): 1674 cm⁻¹ NMR (DMSO-d₆, δ): 2.20-4.40 (17H, m), 3.80 and 3.84 (total 3H, m), 7.19-7.83 (13H, m) MASS (APCI+): 674.1 (M+H)⁺ (free)

### EXAMPLE 19

To a mixture of (4R,9aS)-4-benzhydryl-2-[2-methoxy-5-[5-(trifluoromethyl)-1H-tetrazol-1-yl]benzyl]octahydro-2H-pyrazino[1,2-a]pyrazine trihydrochloride (100 mg), 3-tert-butoxycarbonyl-3-azetidinecarboxylic acid (39.3 ml), 1-hydroxybenzotriazole hydrate (28.8 mg), and triethylamine (79 μl) in dichloromethane (1 ml) was added 1-[3-(dimethylamino)propyl]-3-ethylcarbodiimide hydrochloride (47.6 mg). After stirring at room temperature overnight, the mixture was quenched with aqueous saturated sodium hydrogen carbonate and extracted with dichloromethane. The extract was dried over sodium sulfate and evaporated under reduced pressure. The residue was purified with preparative TLC (methanol/chloroform=1/9) to give an oil (101 mg). To a solution of the oil in a mixture of methanol and dioxane was added 4N hydrogen chloride in dioxane (0.27 ml). After stirring at room temperature overnight, the mixture was evaporated. The residue was added aqueous saturated sodium hydrogen carbonate and extracted with dichloromethane (×3). The organic extracts were dried over magnesium sulfate and evaporated under reduced pressure. The residue was purified with preparative TLC (methanol/chloroform=1/9) to give an oil (38 mg). To a solution of the oil in ethyl acetate (1 ml) were added 4N hydrogen chloride in ethyl acetate (0.2 ml) and hexane (20 ml). After stirring for 30 minutes, the precipitate was collected by filtration and dried under reduced pressure at 50° C. for 5 hours to give (4R, 9aR)-8-(3-azetidinylcarbonyl)-4-benzhydryl-2-[2-methoxy-5-[5-(trifluoromethyl)-1H-tetrazol-1-yl]benzyl]octahydro-2H-pyrazino[1,2-a]pyrazine trihydrochloride (39 mg) as a powder.

IR (KBr): 1651 cm⁻¹ NMR (DMSO-d₆, δ): 2.20-4.40 (20H, m), 3.80 and 3.84 (total 3H, s), 7.10-7.79 (13H, m) MASS (APCI+): 647.2 (M+H)⁺ (free)

### EXAMPLE 20

To a solution of (4R,9aS)-4-benzhydryl-2-[2-methoxy-5-[5-(trifluoromethyl)-1H-tetrazol-1-yl]benzyl]octahydro-2H-pyrazino[1,2-a]pyrazine trihydrochloride (100 mg) in N,N-dimethylformamide (1 ml) were added N,N-diisopropylethylamine (0.129 ml) and dimethylcarbamyl chloride (27.4 μl) at 0° C. After stirring at room temperature for 3 hours, the mixture was quenched with water and extracted with ethyl acetate (×3). The combined extracts were washed with brine, dried over magnesium sulfate, and evaporated under reduced pressure. The residue was purified with preparative TLC (methanol/chloroform=1/19) to give an oil. To a solution of the oil in ethyl acetate (1 ml) were added a solution of 4N hydrogen chloride in ethyl acetate (0.5 ml) and hexane. The precipitate was collected by filteration and dried under reduced pressure for 5 hours at 50° C. to give (4R, 9aR)-4-benzhydryl-8-(dimethylcarbamoyl)-2-[2-methoxy-5-[5-(trifluoromethyl)-1H-tetrazol-1-yl]benzyl]octahydro-2H-pyrazino [1,2-a]pyrazine dihydrochloride (78.7 mg) as a white solid.

mp: 158-164° C. [α]_{D}²⁷: −42.27 (C, 0.125, MeOH) IR (KBr): 1647 cm⁻¹ NMR (DMSO-d₆, δ): 2.20-4.50 (15H, m), 2.71 (6H, s), 3.80 (3H, s), 6.82-7.81 (13H, m) MASS (APCI+): 634.9 (M+H)⁺ (free)

### EXAMPLE 21

To a solution of (4R,9aS)-4-benzhydryl-2-[2-methoxy-5-[5-(trifluoromethyl)-1H-tetrazol-1-yl]benzyl]octahydro-2H-pyrazino [1,2-a]pyrazine trihydrochloride (100 mg) and N,N-diisopropylethylamine (80.2 μl) in ethyl acetate (1 ml) was added methylisocyanate (2 drops). After stirring at room temperature for 1 hour, the mixture was quenched with water and extracted with ethyl acetate (×3). The combined extracts were washed with brine, dried over magnesium sulfate, and evaporated under reduced pressure. The residue was purified with preparative TIC (methanol/chloroform=1/19) to give an oil. To a solution of the oil in ethyl acetate (1 ml) were added 4N hydrogen chloride in ethyl acetate (0.5 ml) and hexane. The precipitate was collected by filteration and dried under reduced pressure for 5 hours at 50° C. to give (4R, 9aR)-4-benzhydryl-2-[2-methoxy-5-[5-(trifluoromethyl)-1H-tetrazol-1-yl]benzyl]-8-(methylcarbamoyl) octahydro-2H-pyrazino [1, 2-a] pyrazine dihydrochloride (88.7 mg) as a white solid.

mp: 160-170° C. [α]_{D}²⁸: −30.27 (C, 0.125, MeOH) IR (KBr): 1647 cm⁻¹ NMR (DMSO-d₆, δ): 2.20-4.50 (18H, m), 3.81 (3H, s), 7.16-7.81 (13H, m) MASS (APCI+): 620.9 (M+H)⁺ (free)

### EXAMPLE 22

To a solution of (4R,9aS)-4-benzhydryl-2-[2-methoxy-5-[5-(trifluoromethyl)-1H-tetrazol-1-yl]benzyl]octahydro-2H-pyrazino[1,2-a]pyrazine trihydrochloride (100 mg) in water (1 ml) and 1N hydrochloric acid (0.3 ml) was added a solution of sodium cyanate (19.3 mg) in water at room temperature, and the mixture was stirred at room temperature for 2 hours. To the mixture was added a solution of sodium cyanate (20 mg) in water and 1N hydrochloric acid (0.3 ml) at room temperature, and the mixture was stirred overnight. To the mixture was added a solution of sodium cyanate (20 mg) in water and 1N hydrochloric acid (0.3 ml) at room temperature, and the mixture was stirred for 2 hours. The mixture was diluted with water and extracted with dichloromethane. The organic extract was dried over sodium sulfate and evaporated under reduced pressure. The residue was purified with preparative TLC (methanol/chloroform=1/19) to give an oil. To a solution of the oil in ethyl acetate (1 ml) were added a solution of 4N hydrogen chloride in ethyl acetate (0.5 ml) and hexane. The precipitate was collected by filtration and dried under reduced pressure for 5 hours at 50° C. to give (4R, 9aR)-4-benzhydryl-8-carbamoyl-2-(2-methoxy-5-(5-(trifluoromethyl)-1H-tetrazol-1-yl]benzyl]octahydro-2H-pyrazino [1,2-a]pyrazine dihydrochloride (80 mg) as a white solid.

mp: 165-190° C. [α]_{D}²⁸: −38.67 (C, 0.125, MeOH) IR (KBr) : 1653 cm⁻¹ NMR (DMSO-₆, δ): 2.20-4.50 (15H, m), 3.81 (3H, s), 7.19-7.81 (13H, m) MASS (APCI+): 606.9 (M+H)⁺ (free)

### EXAMPLE 23

The following compound was obtained according to a similar manner to that of Example 22.

### [[(6R,9aR)-6-Benzhydryl-8-[2-methoxy-5-(5-trifluoromethyl-1H-tetrazol-1-yl]benzyl]octahydro-2H-pyrazino[1,2-a]pyrazin-2-yl]methylene]amine trihydrochloride

IR (KBr) : 1707, 1647, 1512 cm⁻¹ NMR (DMSO-d₆, δ): 2.20-4.40 (19H, m), 7.17-7.85 (13H, m) MASS (APCI+): 591.0 (M+H)⁺ (free)

### EXAMPLE 24

To a solution of (4R,9aS)-4-benzhydryl-2-[2-methoxy-5-[5-(trifluoromethyl)-1H-tetrazol-1-yl]benzyl]octahydro-2H-pyrazino[1,2-a]pyrazine trihydrochloride (100 mg) in ethanol (1 ml) were added methylacetimidate hydrochloride (12 mg) and N,N-diisopropylethylamine (91 μl) at room temperature, and the mixture was allowed to stand at room temperature overnight. To the mixture was added 4N hydrogen chloride in ethyl acetate (0.2 ml), and the mixture was evaporated under reduced pressure. The residue was purified with preparative TLC (methanol/dichloromethane=3/17). The elution was added 4N hydrogen chloride in ethyl acetate, evaporated under reduced pressure, and dried under reduced pressure for 2 hours at 50° C. to give [1-[(6R,9aR)-6-benzhydryl-8-[2-methoxy-5-(5-trifluoromethyl)-1H-tetrazol-1-yl]benzyl]octahydro-2H-pyrazino[1,2-a]pyrazin-2-yl]ethylidene]amine trihydrochloride (85.2 mg) as a white solid.

mp: 191-202° C. [α]_{D}²⁶: −41.67 (C, 0.14, MeOH) IR (KBr): 1682, 1620 cm⁻¹ NMR (DMSO-d₆, δ): 2.21 and 2.27 (total 3H, s), 3.84(3H, brs), 2.20-4.40 (15H, m), 7.18-7.88 (13H, m) MASS (APCI+): 605.1 (M+H)⁺ (free)

### EXAMPLE 25

The following compounds were obtained according to a similar manner to that of Preparation 28.

### (1) (4R,9aS)-4-Benzhydryl-8-(cyclopropylmethyl)-2-[2-methoxy-5-[5-(trifluoromethyl)-1H-tetrazol-1-yl]benzyl]octahydro-2H-pyrazino[1,2-a]pyrazine trihydrochloride

NMR (DMSO-d₆, δ): 0.29-0.36 (2H, m), 0.57-0.61 (2H, m), 1.06 (1H, m), 2.40-4.58 (21H, m), 7.16-7.91 (13H, m), 10.99-11.63 (2H, m) MASS (APCI): 618 (M+H)⁺ (free)

### (2) (4R,9aR)-4-Benzhydryl-8-cyclobutyl-2-[2-methoxy-5-(5-(trifluoromethyl)-1H-tetrazol-1-yl]benzyl]octahydro-2H-pyrazino[1,2-a]pyrazine trihydrochloride

mp: 184-187° C. [α]_{D}^{30.0}: −33.50° (C=1.00, MeOH) IR (KBr): 3404, 1504, 1450, 1265, 1201, 1163 cm⁻¹ NMR (DMSO-d₆, δ): 1.50-4.65 (22H, m), 3.82 (3H, s), 7.10-7.50 (11H, m), 7.70-7.95 (2H, m) MASS (API-ES): 618 (M+H)⁺ (free)

### EXAMPLE 26

A solution of (4R,9aS)-4-benzhydryl-2-[2-methoxy-5-[5-(trifluoromethyl)-1H-tetrazol-1-yl]benzyl]octahydro-2H-pyrazino[1,2-a]pyrazine (150 mg), 3-bromopyridine (42 mg), sodium tert-butoxide (36 mg), tris(dibenzylideneacetone)dipalladium (0) (4.9 mg), and (+)-2,2′-bis(diphenylphosphino)-1,1′-binaphthyl (1.7 mg) in toluene (3 ml) was stirred at room temperature for 10 minutes, followed by 80° C. for 20 hours. After 3-bromopyridine (0.010 ml) and sodium tert-butoxide (14 mg) were added to the solution, the whole was stirred at 80° C. for 2 hours. After being cooled to room temperature, the reaction mixture was poured into water, and extracted with ethyl acetate, and while the aqueous layer was adjusted to pH 9 with aqueous sodium bicarbonate. The extract was dried over sodium sulfate. After removal of solvent by evaporation, the resulting residue was purified by column chromatography on silica gel (8 g) using a mixed solvent of dichloromethane and methanol (35:1). The 35 fractions containing the objective compound were collected and evaporated under reduced pressure to give a syrup. To a solution of the syrup in dichloromethane (3 ml) was added a solution of 4N hydrogenchloride in ethyl acetate (0.25 ml), and triturated with diisopropyl ether. The precipitate was collected by filtration and dried under reduced pressure for 5 hours at 40° C. to give (4R, 9aR)-4-benzhydryl-2-[2-methoxy-5-(5-(trifluoromethyl)-1H-tetrazol-1-yl]benzyl]-8-(3-pyridyl)octahydro-2H-pyrazino[1,2-a]pyrazine trihydrochloride (62 mg) as light brown powder.

mp: 175-178° C. IR (KBr): 3398, 1554, 1510, 1267, 1198, 1163 cm⁻¹ NMR (DMSO-d₆, δ): 2.20-4.60 (15H, m), 3.81 (3H, s), 7.10-7.50 (11H, m), 7.70-7.90 (3H, m), 8.06 (1H, d, J=8.9 Hz), 8.20 (1H, d, J=5.2 Hz), 8.44 (1H, s) MASS (APCI): 641 (M+H)⁺ (free)

### EXAMPLE 27

To a solution of chlorosulfonyl isocyanate (37.4 μl) in dichloromethane (1 ml) was added benzylalcohol (44.4 μl) under 5° C. After the mixture was stirred at the same temperature for 90 minutes under 5° C., and thereto a solution of (4R,9aS)-4-benzhydryl-2-[2-methoxy-5-[5-(trifluoromethyl)-1H-tetrazol-1-yl]benzyl]octahydro-2H-pyrazino[1,2-a]pyrazine (220 mg) and triethylamine (0.11 ml) in dichloromethane (1.5 ml) was added dropwise. The whole mixture was stirred at room temperature for 20 hours. After removal of solvent by evaporation, the resulting residue was purified by column chromatography on silica gel (8 g) using a mixed solvent of dichloromethane and methanol (40:1). The fractions containing the objective compound were collected and evaporated under reduced pressure to give a syrup. A solution of the syrup in mixed solvents of tetrahydrofuran (3 ml) and methanol (3 μl) was hydrogenated over 10% palladium-charcoal (50% wet, 90 mg) at room temperature under atmospheric pressure for 40 minutes. After removal of the catalyst by filtration, the filtrate was evaporated under reduced pressure. The resulting residue was purified by column chromatography on silica gel (7 g) using a mixed solvent of dichloromethane and methanol (35:1). The fractions containing the objective compound were collected and evaporated under reduced pressure. To the residue was added a solution of 4N hydrogen chloride in ethyl acetate (0.10 ml), and triturated with diisopropyl ether. The resulting precipitate was collected by filtration and dried under reduced pressure for 5 hours at 40° C. to give (4R, 9aR)-4-benzhydryl-2-[2-methoxy-5-[5-(trifluoromethyl)-1H-tetrazol-1-yl]benzyl]-8-sulfamoyloctahydro-2H-pyrazino[1,2-a]pyrazine dihydrochloride (95 mg) as as colorless powder.

mp: 174-176° C. [α]_{D}^{30.0}: −39.15° (C=0.295, MeOH) IR (KBr): 3398, 1506, 1458, 1369, 1267, 1201, 1165 cm⁻¹ NMR (DMSO-d₆, δ): 2.10-4.50 (15H, m), 3.84 (3H, s), 6.77 (2H, s), 7.10-7.50 (11H, m), 7.70-7.90 (2H, m) MASS (API-ES): 643 M+H)⁺ (free)

### EXAMPLE 28

To an ice-cooled solution of (4R,9aS)-4-benzhydryl-2-[2-methoxy-5-[5-(trifluoromethyl)-1H-tetrazol-1-yl]benzyl]octahydro-2H-pyrazino[1,2-a]pyrazine (0.3 g), tert-butoxycarbonylglycine (93 mg), 1-hydroxybenzotriazole (72 mg) and triethylamine (0.11 ml) in dichloromethane (25 ml) was added 1-[3-(dimethylamino)propyl]-3-ethylcarbodiimide hydrochloride. After the mixture was stirred for 5 hours at room temperature, additional tert-butoxycarbonylglycine (20 mg), 1-hydroxybenzotriazole (20 mg), and 1-[3-(dimethylamino)propyl]-3-ethylcarbodiimide hydrochloride (25 mg) were added to the mixture. The mixture was stirred further for 15 hours and washed with aqueous sodium carbonate solution, the dichloromethane layer was separated, dried over magnesium sulfate and concentrated under reduced pressure. The syrup was purified by column chromatography on silica gel using a mixed solvent of dichloromethane and methanol (20:1). The fractions containing the objective compound were collected and evaporated under reduced pressure. The resulting syrup was dissolved into ethyl acetate (8 ml) and treated with 4N hydrogen chloride in ethyl acetate (1 ml). After being stirred for 2 hours at room temperature diisopropyl ether (20 ml) was added to the mixture. The resulting precipitate was collected by filtration and washed with diisopropyl ether, dried in vacuo to give white powders of [2-[(6R,9aR)-6-benzhydryl-8-[2-methoxy-5-[5-(trifluoromethyl-1H-tetrazol-1-yl]benzyl]octahydro-2H-pyrazino[1,2-a]pyrazin-2-yl]-2-oxoethyl]amine trihydrochloride (0.38 g)).

IR (KBr): 3400, 2800-2500, 1533 cm⁻¹ NMR (DMSO-d₆, δ): 2.10-4.80 (20H, m), 7.19-7.37 (10H, m), 7.77-7.81 (3H, m), 8.19-8.40 (5H, m) MASS (APCI): 621 (M+H)⁺, 643 (M+Na) (free)

### EXAMPLE 29

The following compound was obtained according to a similar manner to that of Preparation 7.

### (4R,9aR)-8-[(Acetylamino)acetyl]-4-benzhydryl-2-[2-methoxy-5-[5-(trifluoromethyl)-1H-tetrazol-1-yl]benzyl]octahydro-2H-pyrazino[1,2-a]pyrazine dihydrochloride

IR (KBr): 1651, 1512 cm⁻¹ NMR (DMSO-d₆, δ): 1.80 and 1.84 (total 3H, s), 2.20-4.30 (18H, m), 7.18-7.96 (13H, m) MASS (APCI+): 662.93 (M+H)⁺

### EXAMPLE 30

The following compound was obtained according to a similar manner to that of Preparation 31.

### (4R,9aR)-4-Benzhydryl-8-[[(benzyloxyacetyl)amino]acetyl]-2-[2-methoxy-5-[5-(trifluoromethyl)-1H-tetrazol-1-yl]benzyl]octahydro-2H-pyrazino[1,2-a)pyrazine

NMR (CDCl₃, δ): 1.85-2.20 (4H, m), 2.40-3.57 (10H, m), 3.82 (3H, d, J=3.9 Hz), 3.99 (2H, s), 3.98-4.21 (3H, m), 4.59 (2H, s), 6.91-7.53 (18H, m) MASS (ESI+): 769.2 (M+H)⁺, 791.3 (M+Na)

### EXAMPLE 31

To a solution of (4R,9aR)-4-benzhydryl-8-[[(benzyloxyacetyl)amino]acetyl)-2-[2-methoxy-5-[5-(trifluoromethyl)-1H-tetrazol-1-yl]benzyl]octahydro-2H-pyrazino[1,2-a]pyrazine (39 mg) in methanol (15 ml) were added palladium on carbon (10 mg) and concentrated hydrochloric acid (8 ml). After stirring at room temperature under hydrogen for 5 hours, the mixture was filtered. The filtrate was evaporated and purified with preparative TLC (methanol/chloroform=1/9) to give an oil. The oil was added 4N hydrogen chloride in ethyl acetate (0.5 ml), evaporated, dried under reduced pressure at 50° C. for 5 hours to give (4R,9aR)-4-benzhydryl-8-[[(hydroxyacetyl)amino]acetyl]-2-[2-methoxy-5-[5-(trifluoromethyl)-1H-tetrazol-1-yl]benzyl]octahydro-2H-pyrazino[1,2-a]pyrazine dihydrochloride as a white powder (26.3 mg).

IR (KBr): 1684, 1649 cm⁻¹ NMR (DMSO-d₆, δ): 2.20-4.40 (19H, m), 3.81 and 3.83 (total 3H, s), 7.10-7.78 (13H, m) MASS (ESI+): 679.3 (M+H)⁺, 701.2 (M+Na)

### EXAMPLE 32

To a solution of (0.5 g) and triethylamine (0.31 ml) in tetrahydrofuran (10 ml) was added methyl bromoacetate (136 mg) dropwise over 10 minutes, under ice-cooling, and the mixture was stirred at room temperature for 5 hours. The mixture was washed with sodium carbonate aqueous solution, dried over magnesium sulfate and concentrated under reduced pressure. The residue was purified by column chromatography on silica gel using a mixed solvent of dichloromethane and methanol. The fractions containing the objective compound were collected and concentrated under reduced pressure to give methyl [(6R,9aR)-6-benzhydryl-8-[2-methoxy-5-[5-(trifluoromethyl)-1H-tetrazol-1-yl]benzyl]octahydro-2H-pyrazino[1,2-a]pyrazin-2-yl]acetate (0.46 g) as an oil.

NMR (CDCl₃, δ): 1.95-2.25 (5H, m), 2.41 (1H, d, J=11.2 Hz), 2.60-2.80 (4H, m), 2.83 (1H, d, J=10.7 Hz), 3.11 (2H, s), 3.26-3.40 (1H, m), 3.37 (1H, d, J=15.0 Hz), 3.50 (1H, d, J=15.0 Hz), 3.68 (3H, s), 3.80 (3H, s), 4.18 (1H, d, J=7.2 Hz), 6.92 (1H, d, J=8.7 Hz), 7.06-7.30 (11H, m), 7.38 (1H, d, J=2.6 Hz) MASS (APCI): 636 (M+H)⁺

Trihydrochloride of the above compound

IR (KBr): 3400, 2800-2500, 1533 cm⁻¹ NMR (DMSO-d₆, δ): 2.50-5.00 (17H, m), 3.71 (3H, s), 3.81 (3H, s), 7.24-7.33 (11H, m), 7.80-7.85 (2H, m) MASS (APCI): 636 (M+H)⁺ (free)

### EXAMPLE 33

The mixture of methyl [(6R,9aR)-6-benzhydryl-8-[2-methoxy-5-(5-(trifluoromethyl)-1H-tetrazol-1-yl]benzyl]octahydro-2H-pyrazino[1,2-a]pyrazin-2-yl]acetate (0.14 g) and 20% ammonia in methanol was allowed to stand in sealed vessel for 2 days. After removal of solvent, the residue was dissolved into ethyl acetate (5 ml) and thereto 4N hydrogen chloride in ethyl acetate (1 ml) was added. Diisopropyl ether (10 ml) was added to the mixture, and the resulting precipitate was collected by filtration, washed with diisopropyl ether and dried in vacuo to give white powders of 2-[(6R,9aR)-6-benzhydryl-8-(2-methoxy-5-[5-(trifluoromethyl)-1H-tetrazol-1-yl]benzyl]octahydro-2H-pyrazino[1,2-a]pyrazin-2-yl]acetamide trihydrochloride (0.14 g).

IR (KBr): 3400, 2800-2500, 1533 cm⁻¹ NMR (DMSO-d₆, δ): 2.10-4.80 (20H, m), 7.20-8.04 (13H, m), 8.64-9.03 (2H, m) MASS (APCI): 621 (M+H)⁺ (free)

### EXAMPLE 34

The solution of methyl ((6R,9aR)-6-benzhydryl-8-(2-methoxy-5-[5-(trifluoromethyl)-1H-tetrazol-1-yl]benzyl]octahydro-2H-pyrazino[1,2-a]pyrazin-2-yl]acetate (0.14 g) and 24 dimethylamine in tetrahydrofuran (10 ml) was stirred in sealed tube at 40° C. for 2 days. The mixture was concentrated under reduced pressure. The syrup was purified by column chromatography on silica gel using a mixed solvent of hexane and ethyl acetate (4:1). The fractions containing the objective compound were collected and concentrated. The resulting syrup was dissolved into ethyl acetate (8 ml) and treated with 4N hydrogen chloride in ethyl acetate to give 2-[(6R,9aR)-6-benzhydryl-8-[2-methoxy-5-[5-(trifluoromethyl)-1H-tetrazol-1-yl]benzyl]octahydro-2H-pyrazino[1,2-a]pyrazin-2-yl]-N,N-dimethylacetamide trihydrochloride (12 mg).

NMR (DMSO-d₆, δ): 2.10-4.80 (17H, m), 2.87 (3H, s), 2.89 (3H, s), 3.80 (3H, s), 7.23-7.30 (10H, m), 7.77-7.81 (2H, m), 10.0-12.00 (3H, m) MASS (APCI): 649 (M+H)⁺ (free)

### EXAMPLE 35

The following compound was obtained according to a similar manner to that of Example 32.

### (4R,9aR)-4-Benzhydryl-2-[2-methoxy-5-[5-(trifluoromethyl)-1H-tetrazol-1-yl]benzyl]-8-(2-oxopropyl)octahydro-2H-pyrazino[1,2-a]pyrazine trihydrochloride

mp: 175-179° C. [α]_{D}^{30.0}: −43.070 (C=0.70, MeOH) IR (KBr): 3425, 1728, 1506, 1450, 1267, 1199, 1163 cm⁻¹ NMR (DMSO-d₆, δ): 2.14 (3H, s), 3.80 (3H, s), 2.20-4.70 (17H, m), 7.10-7.50 (11H, m), 7.70-7.90 (2H, m) MASS (APCI): 620 (M+H)⁺ (free)

### EXAMPLE 36

A solution of methyl 3-[(6R,9aR)-6-benzhydryl-8-[2-methoxy-5-(5-trifluoromethyl-1H-tetrazol-1-yl]benzyl]octahydro-2H-pyrazino[1,2-a]pyrazin-2-yl]-3-oxopropanoate (80 mg) and potassium carbonate (25 mg) in methanol (1 ml) was stirred at room temperature for 2.5 hours. The mixture was quenched with aqueous saturated ammonium chloride, and the whole solution was evaporated under reduced pressure. The residue was added to dichloromethane and filtered. The filtrate was evaporated to give 3-((6R,9aR)-6-benzhydryl-8-[2-methoxy-5-(5-trifluoromethyl-1H-tetrazol-1-yl]benzyl]octahydro-2H-pyrazino[1,2-a]pyrazin-2-yl)-3-oxopropanoic acid (68 mg) as an oil. MASS (APCI) : 648.87 (M+H)+

### EXAMPLE 37

To a solution of 3-[(6R,9aR)-6-benzhydryl-8-[2-methoxy-5-(5-trifluoromethyl-1H-tetrazol-1-yl]benzyl]octahydro-2H-pyrazino[1,2-a]pyrazin-2-yl)-3-oxopropanoic acid (68 mg), 2 dimethylamine in tetrahydrofuran (78.5 μl), 1-hydroxybenzotriazole hydrate (17 mg) and triethylamine (58.4 μl) in dichloromethane (1 ml) was added 1-[3-(dimethylamino)propyl]-3-ethylcarbodiimide hydrochloride (28 mg) at room temperature. After stirring at room temperature overnight, the mixture was quenched with aqueous saturated sodium hydrogen carbonate and extracted with dichloromethane (×3). The combined organic extracts were dried over sodium sulfate and evaporated under reduced pressure. The residue was purified with preparative TLC (methanol/chloroform=1/9) to give an oil (60 mg). To a solution of the oil in ethyl acetate was added 4N hydrogen chloride in ethyl acetate (0.5 ml). The mixture was evaporated, and dried under reduced pressure at 50° C. for 5 hours to give 2-[(6R, 9aR)-6-benzhydryl-8-[2-methoxy-5-(5-trifluoromethyl-1H-tetrazol-1-yl]benzyl]octahydro-2H-pyrazino[1,2-a]pyrazin-2-yl]-3-oxopropanoic acid N,N-dimethylamide dihydrochloride (57.1 mg) as a powder.

mp: 155-168° C. [α]_{D}²⁶: −25.90 (C, 0.13, MeOH) IR (KBr) 1647 cm⁻¹ NMR (DMSO-d₆, δ): 2.20-4.40 (23H, m), 3.81 (3H, s), 7.10-7.90 (13H, m). MASS (APCI+): 677.2 (M+H)⁺ (free)

### EXAMPLE 38

To a suspension of 1-tert-butoxycarbonylamino-1-cyclopropanecarboxylic acid (71.4 mg), (4R,9aS)-4-benzhydryl-2-[2-methoxy-5-[5-(trifluoromethyl)-1H-tetrazol-1-yl]benzyl]octahydro-2H-pyrazino[1,2-a]pyrazine (200 mg) in dichloromethane (3 ml) were added triethylamine (74.2 μl) and 2-chloro-1-methylpyridinium iodide (136 mg) at room temperature. After being stirred for 3 hours, triethylamine (15 μl) and 2-chloro-1-methylpyridinium iodide (27 mg) were added to the solution at the same temperature, and the whole was stirred at room temperature 20 hours. The solution was poured into aqueous saturated sodium hydrogen carbonate, and extracted with dichloromethane. The organic layer was separated, washed brine, dried over sodium sulfate, and concentrated under reduced pressure. The residue was purified by column chromatography on silica gel (6 g) using a mixed solvent of dichloromethane and methanol (40:1). The fractions containing the objective compound were collected and evaporated under reduced pressure to give a syrup. To a solution of the syrup in dichloromethane (3 ml) was added a solution of 4N hydrogen chloride in ethyl acetate (1.5 ml) under ice-cooling. After the mixture was stirred at room temperature for 2 hours, the solvent was removed by evaporation under reduced pressure. The residue was partitioned between aqueous saturated sodium hydrogen carbonate and dichloromethane, and the organic layer was separated, and dried over sodium sulfate. After removal of the solvent by evaporation, the resulting residue was purified by column chromatography on silica gel (4 g) using a mixed solvent of dichloromethane and methanol (25:1). The fractions containing the objective compound were collected and evaporated under reduced pressure to give a syrup. To a solution of the syrup in dichloromethane (2 ml) was added a solution of 4N hydrogen chloride in ethyl acetate (0.20 ml), and triturated with diisopropyl ether. The precipitate was collected by filteration and dried under reduced pressure for 5 hours at 40° C. to give (4R,9aR)-8-(1-amino-1-cyclopropanecarbonyl)-4-benzhydryl-2-[2-methoxy-5-[5-(trifluoromethyl)-1H-tetrazol-1-yl]benzyl]octahydro-2H-pyrazino[1,2-a]pyrazine trihydrochloride (63 mg) as a colourless powder. IR (KBr): 3433, 2925, 1647, 1504, 1450, 1269, 1203, 1165 cm⁻¹ NMR (DMSO-d₆, δ): 1.10-1.35 (4H, m), 3. 81 (3H, s), 2.20-4.50 (15H, m), 7.10-7.50 (11H, m), 7.70-7.90 (2H, m), 9.06 (3H, S) MASS (APCI): 647 (M+H)⁺ (free)

### EXAMPLE 39

To a suspension of 1-[(tert-butyldimethylsilyloxy)methyl]-1-cyclopropanecarboxylic acid (81.8 mg) and (4R,9aS)-4-benzhydryl-2-[2-methoxy-5-[5-(trifluoromethyl)-1H-tetrazol-1-yl]benzyl]-octahydro-2H-pyrazino[1,2-a]pyrazine (200 mg) in dichloromethane (3 ml) were added triethylamine (74.2 μl) and 2-chloro-1-methylpyridinium iodide (136 mg) at room temperature. After being stirred for 20 hours, the solution was poured into saturated sodium hydrogen carbonate solution, and extracted with dichloromethane. The organic layer was separated, washed brine, dried over sodium sulfate, and concentrated under reduced pressure. The residue was purified by column chromatography on silica gel (6 g) using a mixed solvent of dichloromethane and methanol (60:1). The fractions containing the objective compound were collected and evaporated under reduced pressure to give a syrup (32 mg). To a solution of the syrup in tetrahydrofuran (1 ml) was added tetrabutylammonium fluoride (24 μl) was added under ice-cooling, and the whole was stirred at room temperature for 90 minutes. The reaction mixture was partitioned between water and ethyl acetate, and the organic layer was washed brine, dried over sodium sulfate, and concentrated under reduced pressure. The residue was purified by preparative TLC using a mixed solvent of dichloromethane and methanol (20:1). The bands of silica gel containing the objective compound were collected, and extracted with dichloromethane and methanol (20:1). The extract was evaporated under reduced pressure to give a syrup (10 mg). To a solution of the syrup in dichloromethane (1 ml) was added a solution of AN hydrogen chloride in ethyl acetate (10 μl), and triturated with diisopropyl ether. The precipitate was collected by filtration and dried under reduced pressure for 5 hours at 40° C. to give (4R,9aR)-4-benzhydryl-8-(1-hydroxymethyl-1-cyclopropanecarbonyl)-2-[2-methoxy-5-(5-(trifluoromethyl)-1H-tetrazol-1-yl]benzyl]octahydro-2H-pyrazino[1,2-a]pyrazine dihydrochloride (11 mg) as colorless powder.

IR (KBr): 3398, 1639, 1506, 1460, 1433, 1265, 1199, 1165, 1041 cm⁻¹ NMR (DMSO-d₆, δ): 0.60-1.30 (4H, m), 3.81 (3H, s), 2.20-4.50 (18H, m), 7.10-7.50 (11H, m), 7.70-7.90 (2H, m) MASS (APCI): 662 (M+H)⁺ (free)

### EXAMPLE 40

The following compound was obtained according to a similar manner to that of Example 17.

### (4R, 9aR)-4-Benzhydryl-8-[(2S)-2-[(tert-butyldiphenylsilyl)oxy]propionyl]-2-[2-methoxy-5-[5-(trifluoromethyl)-1H-tetrazol-1-yl]benzyl]octahydro-2H-pyrazino[1,2-a]pyrazine

NMR (CDCl₃, δ): 1.04 (9H, s), 1.28 (3H, d, J=4.7 Hz), 1.80-2.05 (2H, m), 2.20-7.70 (12H, m), 3.81 (3H, s), 3.80-4.20 (2H, m), 4.38-4.55 (1H, m), 6.90-8.11 (23H, m) MASS (APCI+): 874.3 (M+H)⁺, 896.4 (M+Na)

### EXAMPLE 41

To a solution of (4R,9aR)-4-benzhydryl-8-[(2S)-2-[(tert-butyldiphenylsilyl)oxy]propionyl]-2-[2-methoxy-5-[5-(trifluoromethyl)-1H-tetrazol-1-yl]benzyl]octahydro-2H-pyrazino[1,2-a]pyrazine (99.6 mg) in tetrahydrofuran (1.2 ml) were added acetic acid (0.02 ml) and tetrabutylammonium fluoride (1M tetrahydrofuran solution, 0.34 ml) at room temperature. After stirring at room temperature for 6 hours, the mixture was evaporated and purified with preparative TLC (ethyl acetate) to give an oil (78 mg). To a solution of the oil in ethyl acetate (1 ml) was added 4N hydrogen chloride in ethyl acetate (0.5 ml) and hexane (20 ml). After stirring for 30 minutes, the precipitate was collected by filtration and dried under reduced pressure at 50° C. for 5 hours to give (4R,9aR)-4-benzhydryl-8-[(2R)-2-hydroxypropionyl]-2-(2-methoxy-5-[5-(trifluoromethyl)-1H-tetrazol-1-yl]benzyl]octahydro-2H-pyrazino[1,2-a]pyrazine dihydrochloride (68.6 mg) as a white solid.

IR (KBr) : 1651 cm⁻¹ NMR (DMSO-d₆, δ): 2.20-4.40 (19H, m), 7.18-7.82 (13H, m) MASS (APCI+): 636.00 (M+H)⁺

### EXAMPLE 42

To a suspension of 1-acetylamino-1-cyclopropanecarboxylic acid (31.7 mg) in dichloromethane (3 ml) were added triethylamine (46.4 μl) and 2-chloro-1-methylpyridinium iodide (85 mg) at room temperature. After being stirred for 30 minutes, (4R,9aS)-4-benzhydryl-2-[2-methoxy-5-[5-(trifluoromethyl)-1H-tetrazol-1-yl]benzyl]octahydro-2H-pyrazino[1,2-a]pyrazine (125 mg) was added to the solution at the same temperature, and the whole was stirred at room temperature for 14 hours. After removal of solvent by evaporation, to the resulting residue were added N,N-dimethylformamide (3.5 ml) and triethylamine (15 μl), and the whole mixture was heated at 90° C. for 3 hours with stirring. The solution was partitioned between ethyl acetate and water, while aqueous layer was adjusted at pH 9 with aqueous saturated sodium hydrogen carbonate. The organic layer was separated, washed with brine, dried over sodium sulfate, and concentrated under reduced pressure. The resulting residue was purified by column chromatography on silica gel (6 g) using a mixed solvent of toluene and ethyl acetate (35:1). The fractions containing the objective compound were collected and evaporated under reduced pressure to give a syrup. To a solution of the syrup in dichloromethane (3 ml) was added a solution of 4N hydrogen chloride in ethyl acetate (50 Al), and triturated with diisopropyl ether. The precipitate was collected by filtration and dried under reduced pressure for 5 hours at 4020 C. to give (4R,9aR)-8-(1-acetylamino-1-cyclopropanecarbonyl)-4-benzhydryl-2-[2-methoxy-5-(5-(trifluoromethyl)-1H-tetrazol-1-yl]benzyl]octahydro-2H-pyrazino[1,2-a]pyrazine dihydrochloride (47 mg) as colorless powder.

IR (KBr): 3435, 1658, 1649, 1506, 1450, 1265, 1201, 1163 cm⁻¹ NMR (DMSO-d₆, δ): 0.70-0.90 (2H, m), 1.00-1.20 (2H, m), 1.73 (3H, s), 3.82 (3H, s), 2.10-4.50 (15H, m), 7.10-7.50 (11H, m), 7.70-7.90 (2H, m), 8.51 (1H, s) MASS (APCI): 689 (M+H)⁺ (free)

Preparation 33

Methanesulfonyl chloride (22.1 mg) was added to a mixture of (4R, 9aS)-4-benzhydryl-2-[2-methoxy-5-[5-(trifluoromethyl)-1H-tetrazol-1-yl]benzyl]octahydro-2H-pyrazino[1,2-a]pyrazine trihydrochloride (100 mg) and N,N-diisopropylethylamine (116 μl) in dichloromethane under ice-cooling. After being stirred at the same temperature for 2 hours the mixture was poured into ice-water and extracted with ethyl acetate. The extract was washed with brine, dried over magnesium sulphate, and evaporated under reduced pressure. The resulting oil was purified by column chromatography on silica gel using a mixed solvent of dichloromethane and methanol. The fractions containing the objective compound was collected and evaporated under reduced pressure and the resulting residue was treated with 4N hydrogen chloride in ethyl acetate to give (4R,9aR)-4-benzhydryl-2-[2-methoxy-5-[5-(trifluoromethyl)-1H-tetrazol-1-yl]benzyl]-8-(methylsulfonyl)octahydro-2H-pyrazino[1,2-a]pyrazine dihydrochloride (52.8 mg) as colourless powder.

NMR (DMSO-d₆, δ): 2.49-4.31 (23H, m), 7.17-7.80 (13H, m) MASS: (APCI): 642 (M+H)⁺ (free)

### EXAMPLE 43

The following compounds were obtained according to a similar manner to that of Preparation 33.

### (1) (4R,9aR)-4-Benzhydryl-8-dimethylsulfamoyl-2-[2-methoxy-5-[5-(trifluoromethyl)-1H-tetrazol-1-yl)benzyl]octahydro-2H-pyrazino[1,2-a]pyrazine dihydrochloride

mp: 148-152° C. [α]_{D}^{30.0}: −47.80 (C=0.41, MeOH) IR (KBr): 3435, 1506, 1458, 1329, 1267, 1199, 1157 cm⁻¹ NMR (DMSO-d₆, δ): 2.20-4.50 (15H, m), 2.72 (6H, s), 3.84 (3H, s), 7.10-7.50 (11H, m), 7.70-7.90 (2H, m) MASS (APCI): 671 (M+H)⁺ (free)

### (2) (4R,9aR)-4-Benzhydryl-8-[(methylsulfonyl)methylsulfonyl]-2-(2-methoxy-5-[5-(trifluoromethyl)-1H-tetrazol-1-yl]benzyl]octahydro-2H-pyrazino[1,2-a]pyrazine dihydrochloride

mp: 162-168° C. [α]_{D}^{30.0}: −41.13° (C=0.80, MeOH) IR (KBr): 1506, 1458, 1362, 1321, 1165 cm⁻¹ NMR (DMSO-d₆, δ): 3.13 (3H, s), 2.20-4.50 (15H, m), 3.85 (3H, s), 5.25 (2H, s), 7.10-7.50 (11H, m), 7.70-7.90 (2H, m) MASS (API-ES): 720 (M+H)⁺ (free)

### (3) (4R,9aR)-4-Benzhydryl-8-(2-hydroxyethanesulfonyl)-2-[2-methoxy-5-[5-(trifluoromethyl)-1H-tetrazol-1-yl]benzyl]octahydro-2H-pyrazino[1,2-a]pyrazine MASS (APCI+) 671.9 (M+H)⁺

Dihydrochloride of the above compound

IR (KBr): 1512 cm⁻¹ NMR (DMSO-d₆, δ): 2.20-4.40 (19H, m), 3.84 (3H, s), 7.10-7.85 (13H, m) MASS (APCI+): 672.0 (M+H)⁺ (free)

### EXAMPLE 44

To a solution of (4R,9aR)-4-benzhydryl-8-(2-hydroxyethanesulfonyl)-2-(2-methoxy-5-[5-(trifluoromethyl)-1H-tetrazol-1-yl]benzyl]octahydro-2H-pyrazino[1,2-a]pyrazine (16.2 mg) in dichloromethane (1 ml) were added N,N-diisopropylethylamine (8.4 μl) and acetyl chloride (2.6 μl) at room temperature. After stirring for 1 hour, the mixture was quenched with aqueous saturated sodium hydrogen carbonate (10 ml) at 0° C. and extracted with dichloromethane (10 ml×2). The combined organic extracts were washed with brine, dried over magnesium sulfate, and evaporated under reduced pressure. The residue was purified with preparative TLC (methanol/dichloromethane=1/19) to give colorless oil (13.7 mg). To a solution of the oil in ethyl acetate (1 ml) was added 4N hydrogen chloride in ethyl acetate (0.1 ml), and the mixture was evaporated under reduced pressure to give (4R,9aR)-8-(2-acetoxyethanesulfonyl)-4-benzhydryl-2-[2-methoxy-5-[5-(trifluoromethyl)-1H-tetrazol-1-y]benzyl]octahydro-2H-pyrazino[1,2-a]pyrazine dihydrochloride (10 mg) as a white solid.

IR (KBr): 1741 cm⁻¹ NMR (DMSO-d₆, δ): 1.96 (3H, s), 2.20-4.40 (19H, m), 3.84 (3H, s), 7.16-7.83 (13H, m) MASS (APCI+): 714.3 (M+H)⁺, 736.2 (M+Na) (free)

Preparation 34

Diisopropylethylamine (0.236 ml) was added to an ice-cooled solution of 1-[3-(bromomethyl)-4-fluorophenyl)-5-(trifluoromethyl)-1H-tetrazole and in N,N-dimethylformamide (2 ml) and the mixture was stirred for 3 hours at room temperature. The mixture was washed with aqueous sodium hydrogen carbonate. The organic layer was separated, dried over magnesium sulfate, and evaporated under reduced pressure. The syrup was purified by column chromatography on silica gel using a mixed solvent of dichloromethane and methanol (100:1-40:1). The fractions containing the objective compound were collected to give a syrup. The syrup was treated with 4N hydrogen chloride in ethyl acetate solution to give (4R,8as)-4-benzhydryl-2-[2-fluoro-5-(5-(trifluoromethyl)-1H-tetrazol-1-yl]benzyl]octahydropyrrolo[1,2-a]pyrazine dihydrochloride (0.22 g).

IR (KBr): 3400, 2800-2500, 1533 cm⁻¹ NMR (DMSO-d₆, δ): 1.50-5.00 (13H, m), 7.15-8.00 (13H, m), 11.50-12.00 (2H, m) MASS (APCI): 537 (M+H)⁺ (free)

### EXAMPLE 45

The following compound was obtained according to a similar manner to that of Preparation 27.

### (6R, 9aR)-6-Benzhydryl-8-[2-methoxy-5-(5-trifluoromethyl)-tetrazol-1-yl]benzyl]octahydropyrazino[2,1-c][1,4]thiazine dihydrochloride

mp 156-166° C. [α]_{D}²⁸: −57.252 (c=0.131, MeOH) IR (KBr): 3438, 2757-1936, 1508, 1200, 1163 cm⁻¹ NMR (DMSO-d₆, δ): 1.60-4.70 (18H, m), 6.64-7.90 (13H, m), +D₂O MASS (APCI): 581 (M+H)⁺

### EXAMPLE 46

Benzyl 3-oxopropylcarbamate (0.72 g; purity 70-80%; ref; J. Chem. Soc. Chem. Comm., 8, 568 (1988)) and methyl (2R)-6-benzhydryl-4-(2-methoxybenzyl)-2-piperazinecarboxylate (1 g) in tetrahydrofuran (10 ml) were dissolved in a mixture of dichloromethane (10 ml) and acetic acid (280 mg). The whole was stirred for 2 hours at room temperature and thereto sodium triacetoxyborohydride (0.74 g) was added and then the whole was stirred further for 20 hours. The reaction mixture was washed with aqueous saturated sodium carbonate, and the organic layer was dried over magnesium sulfate, and evaporated under reduced pressure. The syrup was purified by column chromatography on silica gel using a mixed solvent of hexane and ethyl acetate (3:1). The main fractions were collected and evaporated under reduced pressure to give methyl (2R)-6-benzhydryl-1-[3-[[(benzyloxy)carbonyl]amino]propyl]-4-(2-methoxybenzyl)-2-piperazinecarboxylate containing the starting material.

MASS (APCI): 622 (M+H)⁺, 431

### EXAMPLE 47

A mixture of methyl (2R)-6-benzhydryl-1-[3-[[(benzyloxy)carbonyl]amino]propyl]-4-(2-methoxybenzyl)-2-piperazinecarboxylate (0.59 g), 10% palladium-charcoal (50% wet, 40 mg) and acetic acid (0.12 ml) in methanol (56 ml) was hydrogenated under 3 atoms for 7.5 hours. After removal of solvent, the resulting syrup was dissolved into dichloromethane (10 ml) and then triethylamine (0.47 ml), and di-tert-butyl dicarbonate (0.5 g) were added to the solution under ice-cooling. After being stirred for 1 hour, the mixture was washed with aqueous sodium carbonate, dried over magnesium sulfate, and evaporated under reduced pressure. The syrup was purified by column chromatography on silica gel using a mixed solvent of hexane and ethyl acetate (4:1). The main fractions were collected and evaporated under reduced pressure to give methyl (2R)-6-benzhydryl-1-[3-[[(tert-butoxy)carbonyl]amino]propyl)-4-(2-methoxybenzyl)-2-piperazinecarboxylate. This compound was dissolved in dichloromethane and the solution was treated with 4N hydrogen chloride in ethyl acetate (5 ml). After removal of solvent by evaporation, the resulting syrup was partitioned between dichloromethane and aqueous sodium carbonate. The organic layer was separated, dried over magnesium sulfate, and evaporated under reduced pressure. The syrup was purified by column chromatography on silica gel using a mixed solvent of dichloromethane: methanol: triethylamine (4:1:0.01). The main fractions were collected and evaporated under reduced pressure to give methyl (2R)-6-benzhydryl-1-(3-aminopropyl)-4-(2-methoxybenzyl)-2-piperazinecarboxylate (240 mg). This compound (240 mg) was dissolved in a mixture of toluene (10 ml) and acetic acid (0.2 ml) and the whole was stirred under reflux for 3 hours. After removal of solvent by evaporation, the resulting syrup was purified by column chromatography on silica gel using a mixed solvent of dichloromethane and methanol (40:1). The fractions containing the objective compound were collected and evaporated under reduced pressure to give (10aR)-4-benzhydryl-2-(2-methoxybenzyl)octahydropyrazino[1,2-a][1,4]diazepin-10(2H)-one (170 mg).

NMR (CDCl₃, δ): 1.80-4.02 (15H, m), 3.72 (3H, s), 5.67 (1H, t like), 6.76-6.89 (2H, m), 7.09-7.41 (12H, m) MASS (APCI): 456 (M+H)⁺ (free)

### EXAMPLE 48

To an ice-cooled solution of (10aR)-4-benzhydryl-2-(2-methoxybenzyl)octahydropyrazino[1,2-a][1,4]diazepin-10(2H)-one (100 mg) in tetrahydrofuran (1 ml) was added lithium aluminum hydride (12.5 mg). The whole was stirred at 50-60° C. for 1 hour, at that time an additional lithium aluminum hydride (36 mg) was added to the mixture, and stirred at 50-60° C. for 5 hours, finally an additional lithium aluminum hydride (10 mg) added, and stirred at 50-60° C. for 5 hours. After cooling with ice, the mixture was treated with 1N sodium hydroxide (5 ml), successively acetyl chloride was added to the whole mixture until the amine spot disappeared on TLC. The reaction mixture was washed with aqueous sodium carbonate, dried over magnesium sulfate, and evaporated under reduced pressure. The syrup was purified by preparative TLC with chloroform: methanol (10:1) to give (10aR)-9-acetyl-4-benzhydryl-2-(2-methoxybenzyl)decahydropyrazino[1,2-a][1,4)diazepine (62 mg).

MASS (APCI): 483 (M+H)⁺

### EXAMPLE 49

A mixture of (10aR)-9-acetyl-4-benzhydryl-2-(2-methoxybenzyl)decahydropyrazino[1,2-a)[1,4)diazepine (60mg) and 1N hydrochloric acid in methanol (2 ml) was hydrogenated over 10% palladium-charcoal (50% wet, 20 mg) at room temperature under 2-3 atoms for 4 days. After removal of the catalyst by filtration, the filtrate was evaporated under reduced pressure to give (10aR)-9-acetyl-4-benzhydryldecahydropyrazino[1,2-a][1,4]diazepine dihydrochloride. To a mixture of this compound, 2-methoxy-5-[5-(trifluoromethyl)-1H-tetrazol-1-yl]benzaldehyde (33 mg) and N,N-diisopropylethylamine (63 μl) in dichloromethane (5 ml) was added sodium triacetoxyborohydride (46 mg). The whole was stirred overnight, and washed with 2N sodium hydroxide. The organic layer was separated, dried over magnesium sulfate and evaporated under reduced pressure. The oil was purified by preparative TLC with hexane: ethyl acetate (2:1). The purified material was treated with 4N hydrogen chloride in ethyl acetate to give (10aR)-9-acetyl-4-benzhydryl-2-[2-methoxy-5-[5-(trifluoromethyl)-1H-tetrazol-1-yl]benzyl]decahydropyrazino[1,2-a][1,4]diazepine dihydrochloride (18 mg).

NMR (DMSO-d₆, δ): 1.74 (3H, s), 3.87 (3H, s), 2.20-5.20 (17H, m), 7.10-7.84 (13H, m), 10.00-10.50 (2H, m) MASS (APCI): 619 (M+H)⁺ (free)

### EXAMPLE 50

The following compound was obtained according to a similar manner to that of Preparation 21.

### (6R, 9aR)-6-Benzhydryl-8-(2-methoxybenzyl)-octahydropyrazino[2,1-c][1,4]thiazine

IR (KBr): 1597, 1495, 1456, 1240, 1113, 1030 cm⁻¹ NMR (CDCl₃, δ): 1.94-2.80 (10H, m), 3.24-3.52 (4H, m), 3.70 (3H, s), 4.23 (1H, d J=6.9 Hz), 6.70-7.32 (14H, m) MASS (APCI): 445 (M+H)⁺

Preparation 35

tert-Butyl (4R,7S,8aS)-4-Benzhydryl-7-hydroxyhexahydropyrrolo[1,2-a]pyrazine-2(1H)-carboxylate (90 mg) was dissolved in 4N hydrogen chloride in ethyl acetate (5.5 ml) and the mixture was stirred at room temperature for 1 hour. The volatile materials were evaporated in vacuo. The residue was partitioned between ethyl acetate and saturated aqueous sodium bicarbonate and the organic phase was washed with brine, dried over magnesium sulfate, and evaporated in vacuo to give (4R,7S,8aS)-4-benzhydryloctahydropyrrolo[1,2-a]pyrazin-7-ol (71.3 mg).

NMR (CDCl₃, δ): 1.86-2.69 (10H, m), 3.01-3.26 (2H, m), 4.03-4.10 (2H, m), 7.13-7.41 (10H, m) MASS (APCI): 309 (M+H)⁺

Preparation 36

The following compounds were obtained according to a similar manner to that of Preparation 35.

### (1) (4R,7R,8aS)-4-Benzhydryloctahydropyrrolo[1,2-a]pyrazin-7-ol

NMR (CDCl₃, δ): 1.61-1.74 (4H, m), 1.95 (1H, dd, J=11.3, 4.0 Hz), 2.36-2.54 (3H, m), 2.70-3.52 (4H, m), 3.92 (1H, d, J=9.48 Hz), 4.13-4.18 (1H, m), 7.12-7.43 (10H, m) MASS (API-ES): 309 (M+H)⁺

(2) (4R, 7S, 8aS)-4-Benzhydryloctahydropyrrolo [1, 2-a]pyrazine-7-carbonitrile NMR (CDCl₃, δ): 2.04-2.82 (8H, m), 2.94-3.31 (3H, m), 3.99-4.17 (2H, m), 7.11-7.43 (10H, m) MASS (APCI): 318 (M+H)⁺

Preparation 37

The following compounds were obtained according to a similar manner to that of Preparation 1.

### (1) N-[(4R, 7R, 8aS)-4-Benzhydryloctahydropyrrolo[1,2-a]pyrazin-7-yl]acetamide dihydrochloride

NMR (DMSO-d₆, δ): 1.71 (3H, s), 2.94-4.45 (13H, m), 7.21-7.52 (10H, m), 8.18 (1H, m), 9.72 (2H, m) MASS (APCI): 350 (M+H)⁺ (free)

### (2) (6R, 9aR)-6-Benzhydryl-2-(3-pyridylcarbonyl)octahydro-2H-pyrazino[1,2-a]pyrazine trihydrochloride MASS (APCI): 413 (M+H)⁺ (free)

Preparation 38

The following compound was obtained according to a similar manner to that of Preparation 19.

### 4-Benzyl 1-tert-butyl (2S)-2-(hydroxymethyl)-1,4-piperazinedicarboxylate

NMR (CDCl₃, δ): 1.46 (9H, s), 2.52 (1H, br), 2.91-3.00 (3H, m), 3.58 (2H, m), 3.84-4.17 (4H, m), 5.15 (2H, s), 7.35-7.45 (5H, m)

Preparation 39

The following compound was obtained according to a similar manner to that of Preparation 20.

### 4-Benzyl-1-tert-butyl (2S)-2-formyl-1,4-piperazinedicarboxylate

MASS (ESI negative): 347 (M−H)

Preparation 40

The following compound was obtained according to a similar manner to that of Preparation 21.

### 4-Benzyl 1-tert-butyl (2R)-2-[[N-(2-methoxybenzyl)-N-(2-oxo-3,3-diphenylpropyl)amino]methyl]-1,4-piperazinedicarboxylate

NMR (CDCl₃, δ): 1.41 (9H, s), 2.70-5.52 (19H, m), 6.73-7.29 (19H, m) MASS (ESI): 678 (M+H)⁺

Preparation 41

The following compound was obtained according to a similar manner to that of Preparation 32.

### Benzyl (6R, 9aR)-6-benzhydryl-8-(2-methoxybenzyl)octahydro-2H-pyrazino[1,2-a]pyrazine-2-carboxylate

NMR (CDCl₃, δ): 1.88 (2H, m), 2.03 (1H, m), 2.49 (2H, m), 2.68 (2H, m), 2.91 (2H, m), 3.28-3.42 (3H, m), 3.67 (3H, s), 3.67-3.78 (2H, m), 4.17 (1H, d, J=5.7 Hz), 5.07 (2H, s), 6.76-6.85 (2H, m), 7.11-7.37 (17H, m) MASS (APCI): 562 (M+H)⁺

Preparation 42

The following compound was obtained according to a similar manner to that of Preparation 18.

### (6R,9aR)-6-Benzhydryloctahydro-2H-pyrazino(1,2-a]pyrazine-2-carboxylate dihydrochloride

[α]_{D}^{23.0}: −60.4411 (C=0.34, MeOH 6.8 mg in 2 ml) mp: 235-236° C. IR (KBr) : 3423, 2588, 2467, 2441, 1703, 1423, 1265, 1230, 1163, 1142, 1049 cm⁻¹ NMR (DMSO-d₆-D₂O, δ): 2.40-3.80 (11H, m), 4.22-4.58 (2H, m), 5.08 (2H, s), 7.14-7.52 (15H, m) MASS (ES+): 442.3 (M+H)⁺ (free)

Preparation 43

To a solution of benzyl (6R,9aR)-6-benzhydryloctahydro-2H-pyrazino(1,2-a]pyrazine-2-carboxylate dihydrochloride (10.01 g) and triethylamine (4.13 g) in dichloromethane (50 ml) was added di-tert-butyl dicarbonate (4.46 g) at room temperature and stirred at the same temperature for 1.5 hours. The mixture was poured into water (50 ml) and the pH of the aqueous layer was adjusted to 5 with 1N hydrochloric acid. The organic layer was separated, washed with brine, dried over sodium sulfate, and concentrated under reduced pressure. The resulting residue was purified by column chromatography on silica gel (120 g) using a mixed solvent of hexane and ethyl acetate (1:3). The fractions containing the objective compound were collected and evaporated under reduced pressure to give 8-benzyl 2-tert-butyl (4R,9aS)-4-benzhydrylhexahydro-2H-pyrazino[1,2-a]pyrazine-2,8(1H)-dicarboxylate (10.6 g) as colorless syrup.

NMR (CDCl₃, δ): 1.32 (9H, br), 1.80-4.20 (13H, m), 5.09 (2H, s), 7.10-7.45 (15H, m) MASS (API-ES): 542 (M+H)⁺

Preparation 44

A solution of 8-benzyl 2-tert-butyl (4R,9aS)-4-benzhydrylhexahydro-2H-pyrazino[1,2-a]pyrazine-2,8(1H)-dicarboxylate (11.0 g) in methanol (110 ml) was hydrogenated over 10% palladium on activated carbon (50% wet, 2.8 g) at room temperature under atmospheric pressure for 4 hours. After removal of the catalyst by filtration, the filtrate was evaporated under reduced pressure to give tert-butyl (4R,9aS)-4-benzhydryloctahydro-2H-pyrazino[1,2-a]pyrazine-2-carboxylate (8.0 g) as an oil.

MASS (API-ES): 408 (M+H)⁺

Preparation 45

The following compound was obtained according to a similar manner to that of Preparation 31.

### tert-Butyl (4R,9aS)-4-benzhydryl-8-[(benzyloxy)acetyl]octahydro-2H-pyrazino[1,2-a]pyrazine-2-carboxylate

NMR (CDCl₃, δ): 1.33 (9H, br s), 1.90-4.30 (15H, m), 4.54-4.57 (2H, m), 7.17-7.34 (15H, m) MASS (ESI): 556 (M+H)⁺

Preparation 46

To a solution of tert-butyl (4R,9aS)-4-benzhydryl-8-[(benzyloxy)acetyl]octahydro-2H-pyrazino[1,2-a]pyrazine-2-carboxylate (499.6 mg) in dichloromethane (2.5 ml) was added trifluoroacetic acid (2.5 ml) at 0° C. Then the mixture was stirred at room temperature for 1.5 hours and evaporated to dryness. The residue was added aqueous saturated sodium bicarbonate (20 ml) and extracted with ethyl acetate (×3). The combined organic extracts were dried over sodium sulfate and evaporated under reduced pressure to give (6R,9aR)-6-benzhydryl-2-[(benzyloxy)acetyl]octahydro-2H-pyrazino[1,2-a]pyrazine (467.6 mg) as an oil.

NMR (CDCl₃, δ): 1.93-4.22 (15H, m), 4.54 (2H, s), 7.11-7.34 (15H, m) MASS (APCI+): 456 (M+H)⁺

Preparation 47

A mixture of (6R,9aR)-6-benzhydryl-2-[(benzyloxy)acetyl]-octahydro-2H-pyrazino[1,2-a]pyrazine (450 mg), 20% palladium hydroxide on carbon (120 mg) and concentrated hydrochloric acid (0.146 ml) in methanol (10 ml) was hydrogenated with 3 atmospheric hydrogen at room temperature for 2 hours. And then to the mixture was added additional 20% palladium hydroxide on carbon (120 mg), and the mixture was hydrogenated under the same condition for 18 hours. The mixture was filtered, and the filtrate was evaporated under reduced pressure to give 2-[(6R,9aR)-6-benzhydryloctahydro-2H-pyrazino[1,2-a)pyrazin-2-yl]-2-oxoethanol dihydrochloride (372.8 mg) as a solid.

NMR (DMSO-d₆, δ): 2.30-5.20 (15H, m), 7.18-7.45 (10H, m) MASS (APCI+): 366 (M+H)⁺ (free)

Preparation 48

2-f(6R,9aR)-6-Benzhydryloctahydro-2H-pyrazino[1,2-a]pyrazin-2-yl]-2-oxoethanol dihydrochloride (200 mg) was partitioned between aqueous saturated sodium bicarbonate and dichloromethane. The organic layer was separated, washed with brine, dried over magnesium sulfate and evaporated under reduced pressure to give 2-[(6R,9aR)-6-benzhydryloctahydro-2H-pyrazino[1,2-a]pyrazin-2-yl]-2-oxoethanol (170 mg) as an oil.

MASS (APCI): 366 (M+H)⁺

Preparation 49

To an ice-cooling mixture of tert-butyl (4R,9aS)-4-benzhydryloctahydro-2H-pyrazino[1,2-a]pyrazine-2-carboxylate (407 mg), triethylamine (0.21 ml) and nicotinic acid (123 mg) in dichloromethane (20 ml) was added 2-chloro-1-methylpyridinium iodide (255 mg), and the whole was stirred at room temperature for 14 hours. The mixture was washed with aqueous sodium bicarbonate and water successively, dried over sodium sulfate and evaporated under reduced pressure. The residue was purified by column chromatography on silica gel using a mixed solvent of dichloromethane and methanol (40:1). The fractions containing the objective compound were collected and concentrated under reduced pressure to give a syrup of tert-butyl (4R,9aS)-4-benzhydryl-8-(3-pyridylcarbonyl)octahydro-2H-pyrazino[1,2-a]pyrazine-2-carboxylate (300 mg).

NMR (CDCl₃, δ): 1.31 (9H, s), 1.50-4.30 (13H, m), 7.10-7.40 (11H, m), 7.70-7.75 (1H, m), 8.61-8.66 (2H, m) MASS (APCI): 535 (M+Na), 513 (M+H)⁺

Preparation 50

The following compound was obtained according to a similar manner to that of Example 42 followed by Preparation 1.

### (6R,9aR)-6-Benzhydryl-2-(2-pyridylcarbonyl)octahydro-2H-pyrazino[1,2-a]pyrazine trihydrochloride

NMR (DMSO-d₆, δ): 2.10-5.20 (13H, m), 7.20-7.70 (12H, m), 7.90-8.00 (1H, m), 8.50-8.55 (1H, m), 9.63 (3H, br s) MASS (APCI): 413 (M+H)⁺ (free)

Preparation 51

To an ice-cooling solution of tert-butyl (4R,9aS)-4-benzhydryloctahydro-2H-pyrazino[1,2-a]pyrazine-2-carboxylate (0.95 g) and triethylamine (0.49 ml) in dichloromethane (20 ml) was added a solution of dimethylcarbamic chloride (0.26 ml) in dichoromethane (4 ml) dropwisely over 30 minutes and the whole was stirred at the same temperature for 1.5 hours. Additional triethylamine (0.5 ml) and dimethylcarbamic chloride (0.26 ml) were added to the mixture and then the whole was stirred for 2 hours. The mixture was washed with aqueous sodium bicarbonate and water successively, dried over sodium sulfate and evaporated under reduced pressure. The residue was purified by column chromatography on silica gel using a mixed solvent of hexane and ethyl acetate (1:1). The fractions containing the objective compound were collected and concentrated under reduced pressure to give an oil of (6R,9aR)-6-benzhydryl-8-(tert-butoxycarbonyl)-N,N-dimethyloctahydro-2H-pyrazino[1,2-a]pyrazine-2-carboxamide (1.07 g).

NMR (CDCl₃, δ), 1.32 (9H, s), 1.80-3.80 (12H, m), 2.80 (6H, s), 4.15 (1H, d, a=7.1 Hz), 7.12-7.30 (10H, m) MASS (APCI): 478 (M+H)⁺, 501 (M+Na)

The oil was treated with 4N hydrogen chloride in ethyl acetate (2.5 ml), the resulting precipitate was collected by filtration, washed with diisopropyl ether, and dried in vacuo to give a powder of (6R,9aR)-6-benzhydryl-N,N-dimethyloctahydro-2H-pyrazino[1,2-a]pyrazine-2-carboxamide dihydrochloride (0.91 g).

NMR (DMSO-d₆, δ): 2.20-4.50 (19H, m), 2.80 (6H, s), 7.20-7.46 (10H, m), 9.50 (2H, br s) MASS (APCI): 379 (M+H)⁺, 401 (M+Na) (free)

Preparation 52

To an ice-cooling solution of tert-butyl (4R,9aS)-4-benzhydryloctahydro-2H-pyrazino[1,2-a]pyrazine-2-carboxylate (1.53 g) and pyridine (0.3 ml) in dichloromethane (40 ml) was added a solution of (1S)-2-chloro-l-methyl-2-oxoethyl acetate (0.522 ml) in dichloromethane (4 ml) dropwisely over 30 minutes, and the whole was stirred at the same temperature for 1.5 hours. Additional (1S)-2-chloro-1-methyl-2-oxoethyl acetate (0.06 ml) and pyridine (0.1 ml) were added to the mixture and the whole was stirred further for 2 hours. The mixture was washed with aqueous sodium bicarbonate and water successively, dried over sodium sulfate and evaporated under reduced pressure. The residue was purified by column chromatography on silica gel using a mixed solvent of hexane and ethyl acetate (9:1). The fractions containing the objective compound were collected and concentrated under reduced pressure to give a white powder of tert-butyl (4R,9aS)-8-[(2S)-2-(acetyloxy)propanoyl)-4-benzhydryloctahydro-2H-pyrazino[1,2a]pyrazine-2-carboxylate (1.62 g).

NMR (CDCl₃, δ): 1.22-1.42 (12H, m), 2.20-2.31 (3H, m), 2.30-4.20 (13H, m), 5.26-5.30 (1H, m), 7.15-7.35 (10H, m) MASS (APCI): 544 (M+Na)⁺, 522 (M+H)⁺

Preparation 53

To an ice-cooling solution of tert-butyl (4R,9aS)-8-[(2S)-2-(acetyloxy)propanoyl)-4-benzhydryloctahydro-2H-pyrazino[1,2-a]pyrazine-2-carboxylate (1.9 g) in methanol (10 ml) was added 1N sodium hydroxide (5.5 ml), and the mixture was stirred at the same temperature for 1.5 hours. The mixture was concentrated under reduced pressure and the residue was partitioned between ethyl acetate and water. The organic layer was separated and dried over magnesium sulfate, and concentrated under reduced pressure. The residue was treated with 4N hydrogen chloride in ethyl acetate (8 ml), the resulting precipitate was collected by filtration, washed with diisopropyl ether, and dried in vacuo to give a powder of (2S)-1-[(6R,9aR)-6-benzhydryloctahydro-2H-pyrazino[1,2-a]pyrazin-2-yl]-1-oxo-2-propanol dihydrochloride (1.59 g).

NMR (DMSO-d₆, δ): 1.14 (3H, d, J=6.2 Hz), 2.20-4.50 (14H, m), 7.20-7.45 (10H, m), 9.36 (1H, br s) MASS (APCI): 380 (M+H)⁺ (free)

Preparation 54

To an ice-cooling mixture of tert-butyl (4R,9aS)-4-benzhydryloctahydro-2H-pyrazino[1,2-a]pyrazine-2-carboxylate (0.5 g) in a mixture of tetrahydrofuran (10 ml) and saturated aqueous sodium bicarbonate was added 3-chloro-3-oxopropyl acetate (0.35 ml) in tetrahydrofuran (2 ml) over 10 minutes. After stirring for 30 minutes at the same temperature, the reaction mixture was extracted with ethyl acetate. The extract was dried over magnesium sulfate and evaporated under reduced pressure. The residue was dissolved into methanol (10 ml) and thereto 1N sodium hydroxide (1.2 ml) and the whole was stirred for 1 hour. After removal of the solvent, the residue was partitioned between water and dichloromethane. The organic layer was separated, dried over magnesium sulfate and evaporated under reduced pressure. The residue was purified by column chromatography on silica gel using a mixed solvent of dichloromethane and methanol (40:1). The fractions containing the objective compound were collected and concentrated under reduced pressure to give an intermediate of tert-butyl (4R,9aS)-8-(3-acetoxypropanoyl)-4-benzhydryloctahydro-2H-pyrazino[1,2-a)pyrazine-2-carboxylate.

NMR (CDCl₃, δ): 1.32 (9H, br s), 1.80-4.30 (20H, m), 7.19-7.30 (10H, m) MASS (API-ES): 524 (M+Na)⁺, 502 (M+H)⁺

The intermediate was treated with 4N hydrogen chloride in dioxane (5 ml), the resulting precipitate was collected by filtration, washed with diisopropyl ether, and dried in vacuo to give powders of 3-[(6R,9aR)-6-benzhydryloctahydro-2H-pyrazino[1,2-a]pyrazin-2-yl]-3-oxo-1-propanol dihydrochloride (0.47 g).

MASS (API-ES): 402 (M+Na)⁺, 380 (M+H)⁺

Preparation 55

The following compound was obtained according to a similar manner to that of preparation 29.

### (R)-2-Benzhydryl-4-benzylpiperazine

mp: 133-135° C. IR (KBr): 1491, 1448, 1138 cm⁻¹ NMR (CDCl₃, δ): 1.86-2.15 (2H, m), 2.57-2.95 (4H, m), 3.28 (1H, d, J=13.0 Hz), 3.46-3.68 (1H, m), 3.56 (1H, d, J=13.0 Hz), 3.83 (1H, d, J=10.5 Hz), 7.05-7.45 (15H, m) MASS (ES+): 365 (M+Na)⁺, 343 (M+H)⁺

Preparation 56

To a solution of (R)-2-benzhydryl-4-benzylpiperazine (4.57 g) in a mixture of acetone (25 ml) and tetrahydrofuran (40 ml) was added triethylamine (2.42 ml) and water (30 ml). Di-tert-butyl dicarbonate (3.49 g) was added to the reaction mixture with water bath cooling and the whole was stirred overnight. Sodium chloride and isopropyl ether were added to the mixture and the organic layer was separated, washed with brine, dried over magnesium sulfate, and evaporated in vacuo. The residue was triturated with hexane to give (R)-2-benzhydryl-4-benzylpiperazine-1-carboxylic acid tert-butyl ester (4.545 g) as a powder. The filtrate was concentrated under reduced pressure and the residue was purified by column chromatography on (silica gel hexane:ethyl acetate (1:0 to 10:1) as eluent) to give the second crop (0.837 g).

mp: 108.5-109° C. IR (KBr): 1687, 1421, 1363, 1172, 1147 cm⁻¹ NMR (CDCl₃, δ): 1.29 and 1.38 (9H, s), 1.90-2.15 (2H, m), 2.55-4.05 (6H, m), 4.70-5.06 (2H, m), 7.02-7.52 (15H, m) MASS (ES+): 466 (M+Na)⁺443 (M+H)⁺

Preparation 57

To a solution of (R)-2-benzhydryl-4-benzylpiperazine-1-carboxylic acid tert-butyl ester (5.30 g) in a mixture of tetrahydrofuran (53 ml) and methanol (53 ml) was added 10% palladium hydroxide on carbon (0.53 g) and the mixture was hydrogenated with 3 atmospheric hydrogen at 40° C. for 20 hours. After cooling, the mixture was filtered and the filtrate was evaporated in vacuo to give (R)-2-benzhydrylpiperazine-1-carboxylic acid tert-butyl ester (4.49 g).

mp: 100-105° C. IR (KBr): 16769, 1412, 1169, 1097 cm⁻¹ NMR (CDCl₃, É-): 1.28 and 1.43 (9H, s), 2.55-4.05 (6H, m), 5.70-5.10 (2H, m), 7.05-7.50 (10H, m) MASS (APCI): 343 (M+H)⁺ MASS (ES+): 375 (M+Na)⁺, 353 (M+H)⁺, 297 (M-tBu)⁺

Preparation 58

To a solution of 2,6-dimethoxy-3-nitrobenzoic acid (156.15 g) and methyl iodide (66 ml) in N,N-dimethylformamide (460 ml) was added potassium carbonate (142 g) portionwise with water bath cooling. After 3 hours of stirring, the mixture was poured into ice-water (4.51 ml) and the whole was stirred for 3 hours. The resulting precipitates were collected by filtration, washed with water, and dried to give methyl 2,6-dimethoxy-3-nitrobenzoate (164.73 g).

mp: 77-78° C. IR (KBr): 1739, 1593, 1522, 1354, 1300, 1263, 1117, 1086 cm⁻¹ NMR (CDCl₃, δ): 3.90 (3H, s), 3.94 (3H, s), 3.95 (3H, s), 6.76 (1H, d, J=9.3 Hz), 8.09 (1H, d, J=9.3 Hz) MASS (ES+): 264 (M+Na)⁺

Preparation 59

The following compounds were obtained according to a similar manner to that of Preparation 58.

### (1) Methyl 3-chloro-2,6-dimethoxy-5-nitrobenzoate

NMR (CDCl₃, δ): 3.95 (3H, s), 3.98 (6H, s), 8.06 (1H, s) MASS (ESI+): 298 (M+Na)

### (2) Methyl 2,4-dichloronicotinate

NMR (CDCl₃, δ): 4.00 (3H, s), 7.33 (1H, d, J=5.38 Hz), 8.35 (1H, d, J=5.36 Hz)

Preparation 60

A solution of 2,6-dimethoxy-3-nitrobenzoic acid methyl ester (5.0 g) in a mixture of methanol (25 ml) and tetrahydrofuran (25 ml) was hydrogenated with 10% palladium on carbon (50% wet, 0.5 g) for 2 days. The mixture was filtered and evaporated in vacuo to give 3-amino-2,6-dimethoxybenzoic acid methyl ester (4.462 g).

mp: 78-80° C. IR (ATR): 3457, 3365, 1712, 1494, 1255, 1081 cm⁻¹ NMR (CDCl₃, δ): 3.75 (3H, s), 3.82 (3H, s), 3.93 (3H, s), 6.55 (1H, d, J=8.7 Hz), 6.74 (1H, d, J=8.7 Hz) MASS (ES+): 234 (M+Na)⁺, 212 (M+H)⁺

Preparation 61

The following compound was obtained according to a similar manner to that of Preparation 60.

### Methyl 5-amino-2,4-dimethoxynicotinate

NMR (CDCl₃, δ): 3.48 (1H, br s), 3.87 (3H, s), 3.90 (3H, s), 3.92 (3H, s), 7.66 (1H, s) MASS (API-ES): 213 (M+H)⁺

Preparation 62

To a solution of 3-amino-2,6-dimethoxybenzoic acid methyl ester (4.41 g) and triethylamine (3.8 ml) in methylene chloride (45 ml) was added dropwise a solution of trifluoroacetic anhydride (3.54 ml) in methylene chloride (3.5 ml) with ice salt bath cooling. After stirring for 0.5 hour, water was added to the mixture. The organic layer was separated, washed with brine, dried over magnesium sulfate and silica gel (19.2 g), and evaporated in vacuo to give 2,6-dimethoxy-3-[(trifluoroacetyl)amino]benzoic acid methyl ester (5.20 g). mp: 112-113° C. IR (KBr) : 1739, 1593, 1522, 1354, 1300, 1263, 1117, 1086 cm⁻¹ NMR (CDCl₃, δ): 3.84 (3H, s), 3.88 (3H, s), 3.95 (3H, s), 6.71 (1H, d, J=9.1 Hz), 8.26 (1H, d, J=9.1 Hz), 8.32 (1H, brs) MASS (ES+): 330 (M+Na)⁺, 308 (M+H)⁺

Preparation 63

The following compounds were obtained according to a similar manner to that of Preparation 62.

### (1) Methyl 2,4-dimethoxy-5-[(trifluoroacetyl)amino]nicotinate

NMR (CDCl₃, δ): 3.95 (3H, s), 3.96 (3H, s), 4.01 (3H, s), 8.15 (1H, br s), 8.98 (1H, s) MASS (API-ES): 309 (M+Na)⁺

### (2) Methyl 2,6-diethoxy-3-[(trifluoroacetyl)amino]benzoate

NMR (CDCl₃, δ): 1.41 (3H, t, J=7.0 Hz) 3.93 (6H, s), 4.04 (4H, q, J=7.0 Hz), 6.69 (1H, d, J=9.18 Hz), 8.23 (1H, d, J=9.10 Hz), 8.40 (1H, br s) MASS (API-ES): 358 (M+Na)⁺

### (3) Ethyl 6-methoxy-2-methyl-3-[(trifluoroacetyl)amino]benzoate

NMR (CDCl₃, δ): 1.38 (3H, t, J=7.1 Hz), 2.16 (3H, s), 3.83 (3H, s), 4.40 (2H, q, J=7.1 Hz), 6.81 (1H, d, J=8.9 Hz), 7.57 (1H, d, J=8.9 Hz) MASS (ESI+): 306.38 (M+H)⁺

### (4) Methyl 3-chloro-2,6-dimethoxy-5-((trifluoroacetyl)-amino]benzoate

NMR (CDCl₃, δ): 3.89 (3H, s), 3.89 (3H, s), 4.00 (3H, s), 8.38 (1H, s) MASS (ESI+): 364 (M+Na)

Preparation 64

A solution of 2,6-dimethoxy 3-[(trifluoroacetyl)amino]benzoic acid methyl ester (5.09 g) in carbon tetrachloride (60 ml) was added triphenylphosphine (6.74 g) and the whole was refluxed for 15 hours. After cooling, diisopropyl ether (60 ml) was added and the mixture was stirred for 1 hour with ice bath cooling. The resulting precipitates were removed by filtration and the filtrate was evaporated in vacuo. The residue was dissolved in N,N-dimethylformamide (37 ml) and sodium azide (3.23 g) was added to the solution with water bath cooling. After stirring for 1 hour, the mixture was poured into a mixture of ice water (180 ml) and ethyl acetate (100 ml). The organic layer was separated, dried over magnesium sulfate, and evaporated in vacuo. The residue was triturated with diisopropyl ether (20 ml) and the resulting precipitates were removed by filtration and the filtrate was evaporated in vacuo. The residue was dissolved in methylene chloride and the solution was treated with silica gel to give methyl 2,6-dimethoxy-3-[5-(trifluoromethyl)-1H-tetrazol-1-yl]benzoate (5.08 g)).

IR (ATR): 1735, 1598, 1494, 1309, 1261, 1163, 1075 cm⁻¹ NMR (CDCl₃, δ): 3.65 (3H, s), 3.94 (3H, s), 3.96 (3H, s), 6.81 (1H, d, J=8.9 Hz), 7.38 (1H, d, J=7.9 Hz) MASS (ES+): 355 (M+Na)⁺, 333 (M+H)⁺

Preparation 65

The following compounds were obtained according to a similar manner to that of Preparation 64.

### (1) Methyl 2,4-dimethoxy-5-[5-(trifluoromethyl)-1H-tetrazol-1-yl]nicotinate

NMR (CDCl₃, δ): 3.85 (3H, s), 3.97 (3H, s), 4.08 (3H, s), 8.13 (1H, s) MASS (API-ES): 356 (M+Na)⁺

### (2) Methyl 2,6-diethoxy-3-[5-(trifluoromethyl)-1H-tetrazol-1-yl]benzoate

NMR (CDCl₃, δ): 1.03 (3H, t, J=7.0 Hz), 1.44 (3H, t, J=7.0 Hz), 3.80 (2H, q, J=7.0 Hz), 3.93 (3H, s), 4.15 (2H, q, J=7.0 Hz), 6.79 (1H, d, J=8.96 Hz), 7.35 (1H, d, J=8.94 Hz) MASS (API-ES): 383 (M+Na)⁺

### (3) Ethyl 6-methoxy-2-methyl-3-[5-(trifluoromethyl)-1H-tetrazol-1-yl]benzoate

NMR (CDCl₃, δ): 1.40 (3H, t, J=7.2 Hz), 1.92 (3H, s), 3.93 (3H, s), 4.43 (2H, q, J=7.2 Hz), 6.95 (1H, d, J=8.9 Hz), 7.29 (1H, d, J=8.9 Hz)

### (4) Methyl 3-chloro-2,6-dimethoxy-5-[5-(trifluoromethyl)-1H-tetrazol-1-yl]benzoate

NMR (CDCl₃, δ): 3.64 (3H, s), 3.99 (3H, s), 4.02 (3H, s), 7.51 (1H, s) MASS (ESI+): 389.1 (M+Na)

Preparation 66

A solution of methyl 2,6-dimethoxy-3-[5-(trifluoromethyl)-1H-tetrazol-1-yl]benzoate (0.8 g) in toluene (13 ml) was added a solution of diisobutyl aluminum hydride (1.01N in toluene, 6.2 ml) with ice salt bath cooling under nitrogen atmosphere and the mixture was stirred for 20 minutes. The mixture was made acidic by diluted hydrochloric acid and was extracted with ethyl acetate. The extract was washed with brine, dried over magnesium sulfate, and evaporated in vacuo. The residue was triturated with a mixture of petroleum ether and diisopropyl ether and the resulting precipitate was collected by filtration, and dried to give [2,6-dimethoxy-3-[5-(trifluoromethyl)-1H-tetrazol-1-yl]phenyl]methanol (586 mg). mp: 74-79° C. IR (KBr) : 3477, 1597, 1539, 1498, 1198, 1169, 1088 cm⁻¹ NMR (CDCl₃, δ): 2.35 (1H, br s), 3.64 (3H, s), 3.99 (3H, s), 4.78 (2H, s), 6.86 (1H, d, J=8.9 Hz), 7.32 (1H, d, J=8.9 Hz) MASS (ES+): 327 (M+Na)⁺, 305 (M+H)⁺

Preparation 67

The following compounds were obtained according to a similar manner to that of Preparation 66.

### (1) [2,4-Dimethoxy-5-[5-(trifluoromethyl)-1H-tetrazol-1-yl]-3-pyridyl]methanol

NMR (CDCl₃, δ): 3.76 (3H, s), 2.31 (1H, t, J=6.82 Hz), 4.10 (3H, s), 4.75 (2H, d, J=6.78 Hz), 8.12 (1H, s) MASS (API-ES): 328 (M+Na)⁺

### (2) [2,6-Diethoxy-3-[5-(trifluoroethyl)-1H-tetrazol-1-yl]phenyl]methanol

NMR (CDCl₃, δ): 1.80 (3H, t, J=7.0 Hz), 1.52 (3H, t, J=7.0 Hz), 3.77 (2H, q, J=7.0 Hz), 4.20 (2H, q, J=7.0 Hz), 4.78 (2H, br s), 6.82 (1H, d, J=8.92 Hz), 7.30 (1H, d, J=8.82 Hz) MASS (API-ES): 355 (M+Na)⁺

### (3) [6-Methoxy-2-methyl-3-[5-(trifluoromethyl)-1H-tetrazol-1-yl]phenyl]methanol

NMR (CDCl₃, δ): 2.01 (3H, s), 3.95 (3H, s), 4.82 (2H, s), 6.93 (1H, d, J=4.4 Hz), 7.22 (1H, d, J=4.4 Hz)

### (4) [3-Chloro-2,6-dimethoxy-5-[5-(trifluoromethyl)-1H-tetrazol-1-yl]phenyl]methanol

NMR (CDCl₃, δ): 3.62 (3H, s), 4.07 (3H, s), 4.80 (2H, d, J=6.3 Hz), 7.45 (1H, s)

Preparation 68

To a solution of [2,6-dimethoxy-3-[5-(trifluoromethyl)-1H-tetrazol-1-yl]phenyl]methanol in methylene chloride (14 ml) was added 4-methylmorpholine N-oxide (755 mg) and molecular sieves 4A (2.8 g), and the mixture was stirred at room temperature for 20 minutes. Tetrapropylammonium perruthenate (97 mg) was added to the mixture and the whole was stirred for 1 hour. The mixture was filtered through cerite and silica gel (45 g) and washed with ethyl acetate. The filtrate and washings were combined, evaporated in vacuo to give crude 2,6-dimethoxy-3-[5-(trifluoromethyl)-1H-tetrazol-1-yl]benzaldehyde (1.123 g). It was triturated with diisopropyl ether, collected by filtration, and dried to give pure one (971 mg).

mp: 145-147° C. IR (KBr): 1689, 1599, 1495, 1406, 1184, 1090 cm⁻¹ NMR (CDCl₃, δ): 3.71 (3H, s), 4.04 (3H, s), 6.94 (1H, d, J=9.0 Hz), 7.55 (1H, d, J=9.0 Hz), 10.48 (1H, s) MASS (ES+): 357 (M+Na+MeOH)⁺, 325 (M+Na)⁺, 393 (M+H)⁺

Preparation 69

The following compounds were obtained according to a similar manner to that of Preparation 68.

### (1) 2,4-Dimethoxy-5-[5-(trifluoromethyl)-1H-tetrazol-1-yl]nicotinaldehyde

NMR (CDCl₃, δ): 3.86 (3H, s), 4.16 (3H, s), 8.29 (1H, s), 10.41 (1H, s)

### (2) 6-Methoxy-2-methyl-3-[5-(trifluoromethyl)-1H-tetrazol-1-yl]benzaldehyde

NMR (CDCl₃, δ): 2.15 (3H, s), 4.04 (3H, s), 7.06 (1H, d, J=8.9 Hz), 7.43 (1H, d, J=8.9 Hz), 10.65 (1H, s)

### (3) 3-Chloro-2,6-dimethoxy-5-[5-(trifluoromethyl)-1H-tetrazol-1-yl]benzaldehyde

NMR (CDCl₃, δ): 3.70 (3H, s), 4.10 (3H,s), 7.69 (1H, s), 10.40 (1H, s) MASS (ESI+): 391.2 (M+Na+MeOH)

Preparation 70

To an ice-cooled solution of [2,6-dimethoxy-3-[5-(trifluoromethyl)-1H-tetrazol-1-yl]phenyl]methanol (329 mg) in chloroform (5 ml) was added phosphorus tribromide (0.111 ml) in dichloromethane (1 ml) over 5 minutes and the whole was stirred at the same temperature for 15 minutes followed by room temperature for 10 minutes. The mixture was poured into ice-cooled aqueous sodium bicarbonate and the organic layer was separated, dried over magnesium sulfate, and evaporated under reduced pressure. The residue was purified by column chromatography on silica gel using a mixed solvent of hexane and ethyl acetate (4:1). The fractions containing the objective compound were collected and evaporated under reduced pressure to give crystals of 1-[3-(bromomethyl)-2,4-dimethoxyphenyl]-5-(trifluoromethyl)-1H-tetrazole (280 mg). mp: 89.5-90.0° C. NMR (DMSO-d₆, δ): 3.66 (3H, s), 4.02 (3H, s), 4.62 (2H, s), 6.85 (1H, d, J=8.9 Hz), 7.33 (1H, d, J=8.9 Hz) MASS (API-ES): 391 (Na+M+2)⁺, 389 (Na+M)⁺, 341

Preparation 71

To a solution of diisopropylamine (1.21 g) in tetrahydrofuran (9 ml) was added dropwise 1.56 M solution of butyllithium in hexane (7.0 ml) below −65° C. and the mixture was stirred at −78° C. for 20 minutes. To this solution was added dropwise 2,4-dichloropyridine (1.47 g) in tetrahydrofuran (8 ml) at −78° C. and the resulting mixture was stirred at the same temperature for 20 minutes. Dry ice and iodamethane were added successively and the reaction mixture was allowed to warm to room temperature over 1 hour. The reaction was quenched with water. The aqueous phase was washed with ethyl acetate, acidified to pH 2 with 1N hydrochloric acid, and extracted twice with ethyl acetates. The combined extracts were washed with brine, dried over magnesium sulfate, and concentrated under reduced pressure to give 2,4-dichloronicotinic acid (1.45 g).

NMR (CD₃OD, δ): 7.56 (1H, d, J=5.46), 8.37 (1H, d, J=5.44 Hz)

Preparation 72

A mixture of methyl 2,4-dichloronicotinate (90 mg) and sodium methoxide (283 mg) in methanol (1 ml) was heated to reflux for 6 hours. The volatile materials were evaporated under reduced pressure and the residue was partitioned between ethyl acetate and 1N hydrochloric acid. The organic phase was washed with brine, dried over magnesium sulfate, and evaporated under reduced pressure to give methyl 2,4-dimethoxynicotinate (76 mg).

NMR (CDCl₃, δ): 3.87 (3H, s), 3.91 (3H, s), 3.95 (3H, s), 6.04 (1H, d, J=6.04 Hz), 8.10 (1H, d, J=6.00 Hz) MASS (APCI): 198 (M+H)⁺

Preparation 73

To a solution of nitronium tetrafluoroborate (343 mg) in sulfolane (1 ml) was added methyl 2,4-dimethoxynicotinate (34 mg) and the mixture was heated at 80° C. for 3 hours. After being cooled to room temperature, the reaction mixture was diluted with ethyl acetate, washed with water and brine, dried over magnesium sulfate, and evaporated under reduced pressure. The residue was purified by flash chromatography on silica gel eluting with ethyl acetate-hexane (1:4) to give methyl 2,4-dimethoxy-5-nitronicotinate (9.0 mg).

NMR (CDCl₃, δ): 3.95 (3H, s), 4.10 (3H, s), 4.04 (3H, s), 8.80 (1H, s) MASS (API-ES): 243 (M+H)⁺

Preparation 74

To a solution of methyl 2,6-diethoxybenzoate (224 mg) in dichloromethane (2 ml) was added sulfuric acid (216 mg) at −20° C. followed by dropwise addition of nitric acid (69.2 mg) at the same temperature. The reaction mixture was allowed to warm to 0° C. over 1 hour. Water was carefully added and the mixture was extracted twice with ethyl acetates. The combined extracts were washed with brine, dried over magnesium sulfate, and concentrated under reduced pressure. The residue was purified by column chromatography on silica gel eluting with ethyl acetate-hexane (1:8) to give methyl 2,6-diethoxy-3-nitrobenzoate (113 mg).

NMR (CDCl₃, δ): 1.33-1.50 (6H, m), 3.93 (3H, s), 3.99-4.20 (4H, m), 6.71 (1H, d, J=9.34 Hz), 8.05 (1H, d, J=9.36 Hz) MASS (API-ES): 292 (M+Na)⁺

Preparation 75

A a solution of methyl 2,6-diethoxy-3-nitrobenzoate (1.56 g) in ethyl acetate (16 ml) was hydrogenated over platinum oxide (78.9 mg) at room temperature for 3 hours. After the catalyst was removed by filtration, the filtrate was concentrated under reduced pressure to give methyl 3-amino-2,6-diethoxybenzoate (1.34 g).

NMR (CDCl₃, δ): 1.22-1.38 (6H, m), 3.91 (3H, s), 3.94-4.13 (4H, m), 6.53 (1H, d, J=8.70 Hz), 6.72 (1H, d, J=8.76 Hz) MASS (API-ES): 240 (M+H)⁺

Preparation 76

To a solution of [2,6-diethoxy-3-[5-(trifluoroethyl)-1H-tetrazol-1-yl]phenyl]methanol (88.8 mg) in dimethyl sulfoxide (1 ml) was added a mixture of sulfur trioxide pyridine complex (170 mg) and triethylamine (216 mg) in dimethyl sulfoxide (1 ml), and the mixture was stirred at room temperature for 0.5 hour. The reaction mixture was quenched with saturated aqueous sodium bicarbonate and extracted three times with ethyl acetates. The combined extracts were washed with brine, dried over magnesium sulfate, and concentrated under reduced pressure to give 2,6-diethoxy-3-[5-(trifluoromethyl)-1H-tetrazol-1-yl]benzaldehyde (88.3 mg).

NMR (CDCl₃, δ): 1.06 (3H, t, J=7.0 Hz), 1.54 (3H, t, J-7.0 Hz), 3.90 (2H, q, J=7.0 Hz), 4.25 (2H, q, J=7.0 Hz), 6.90 (1H, d, J=9.02 Hz), 7.53 (1H, d, J=8.98 Hz), 10.49 (1H, s) MASS (API-ES): 353 (M+Na)⁺

Preparation 77

To a solution of ethyl 2-methoxy-6-methylbenzoate (250 mg) in carbon tetrachloride (10 ml) was added dropwise bromine (66.3 μl) at room temperature. After stirring at room temperature overnight, the mixture was poured into a mixture of water and ethyl acetate and separated. The organic layer was separated and the aqueous layer was extracted with ethyl acetate. The combined organic layers were washed with water and brine, dried over magnesium sulfate and evaporated under reduced pressure to give ethyl 3-bromo-6-methoxy-2-methylbenzoate (343.5 mg) as an oil.

NMR (CDCl₃, δ): 1.36 (3H, t, J=7.1 Hz), 2.32 (3H, s), 3.80 (3H; s), 4.40 (2H, q, J=7.1 Hz), 6.66 (1H, d, J=8.9 Hz), 7.50 (1H, d, J=8.9 Hz) MASS (ESI+): 295 and 297 (M+Na)⁺

Preparation 78

A mixture of ethyl 3-bromo-6-methoxy-2-methylbenzoate (2.1 g), benzophenone imine (1.55 ml), sodium tert-butoxide (1.03 g), tris(dibenzylideneacetone)dipalladium (1.76 g), and (RS)-2,2′-bis(diphenylphosphino)-1,1′-binaphthyl (3.59 g) in toluene (20 ml) was stirred under nitrogen atmosphere at 90° C. overnight. The mixture was quenched with water and the whole was extracted with ethyl acetate (×3). The combined organic layers were washed with brine, dried over magnesium sulfate, and evaporated under reduced pressure. The residue was purified by column chromatography (silica gel (250 ml), ethyl acetate/hexane (1/9)) to give 2.15 g of a yellow oil. To the solution of the oil in tetrahydrofuran (35 ml) was added 1N hydrochloric acid (30 ml) at room temperature, and the mixture was stirred at room temperature for 1 hour. The mixture was quenched with a mixture of aqueous saturated sodium bicarbonate and brine, and the whole was extracted with ethyl acetate (×3). The combined organic layers were washed with brine, dried over magnesium sulfate, evaporated under reduced pressure. The residue was purified by column chromatography (silica gel (150 ml), ethyl acetate/hexane (1:1)) to give ethyl 3-amino-6-methoxy-2-methylbenzoate (884 mg) as an oil.

NMR (CDCl₃, δ): 1.38 (3H, t, J=7.2 Hz), 2.06 (3H, s), 3.75 (3H, s), 4.40 (2H, q, J=7.2 Hz), 6.67 (1H, d, J=8.8 Hz), 6.69 (1H, d, J=8.8 Hz) MASS (ESI+): 210.18 (M+H)⁺, 251.2 (M+H+MeCN)⁺

Preparation 79

Methyl 3-chloro-2,6-dimethoxy-5-nitrobenzoate (5.0 g) was added to a mixture of iron powder (5.37 g) and ammonium chloride (0.61 g) in ethanol and water, and the mixture was stirred under reflux for 1 hour. After cooling, the mixture was filtered and evaporated. The residue was dissolved in ethyl acetate, washed with water and brine, dried over magnesium sulfate, and evaporated to give methyl 3-amino-5-chloro-2,6-dimethoxybenzoate (3.97 g) as an oil.

NMR (CDCl₃, δ): 3.79 (3H, s), 3.81 (3H, s), 3.94 (3H, s), 6.79 (1H, s) MASS (ESI+): 268 (M+Na)⁺

### EXAMPLE 51

The following compounds were obtained according to a similar manner to that of Preparation 34.

### (1) (4R, 9aR)-8-Acetyl-4-benzhydryl-2-[2,6-dimethoxy-3-[5-(trifluoromethyl)-1H-tetrazol-1-yl]benzyl]octahydro-2H-pyrazino[1,2-a]pyrazine dihydrochloride

NMR (DMSO-d₆, δ): 1.91-1.96 (3H, m), 2.10-4.50 (21H, m), 7.10-7.85 (12H, m) MASS (APCI): 636 (M+H)⁺ (free)

### (2) 3-[(6R,9aR)-6-Benzhydryl-8-[2,6-dimethoxy-3-[5-(trifluoromethyl)-1H-tetrazol-1-yl) benzyl]octahydro-2H-pyrazino[1,2-a]pyrazin-2-yl]-3-oxo-1-propanol dihydrochloride

NMR (DMSO-d₆, δ): 3.77 (3H, s), 2.20-4.60 (23H, m), 7.05-7.45 (11H, m), 7.84 (1H, d, J=9.0 Hz) MASS (API-ES): 666 (M+H)⁺ (free)

### EXAMPLE 52

The following compound was obtained according to a similar manner to that of Preparation 27.

### (1) (4R, 9aR)-4-Benzhydryl-2-[2,6-dimethoxy-3-[5-(trifluoromethyl)-1H-tetrazol-1-yl]benzyl]-8-(3-pyridylcarbonyl)octahydro-2H-pyrazino[1,2-a]pyrazine trihydrochloride

NMR (DMSO-d₆, δ): 2.10-5.00 (21H, m), 7.00-8.15 (16H, m), 8.55 (1H, d, J=2.8 Hz) MASS (APCI): 699 (M+H)⁺ (free)

### (2) (4R, 9aR)-4-Benzhydryl-2-[2,6-dimethoxy-3-[5-(trifluoromethyl)-1H-tetrazol-1-yl]benzyl]-8-(2-pyridylcarbonyl)octahydro-2H-pyrazino[1,2-a]pyrazine trihydrochloride

NMR (DMSO-d₆, δ): 2.10-5.00 (21H, m), 7.00-8.15 (16H, m), 8.50-8.55 (1H, m), 9.00-10.50 (3H, m) MASS (APCI): 721 (M+Na), 699 (M+H)⁺ (free)

### (3) (2S)-1-[(6R, 9aR)-6-Benzhydryl-8-[2,6-dimethoxy-3-[5-(trifluoromethyl)-1H-tetrazol-1-yl]benzyl]octahydro-2H-pyrazino[1,2-a]pyrazin-2-yl]-1-oxo-2-propanol dihydrochloride

NMR (DMSO-d₆, δ): 1.13 (3H, d, J=5.6 Hz), 1.90-4.52 (22H, m), 7.08-7.86 (12H, m) MASS (APCI+): 666.13 (M+H)⁺

### (4) 2-[(6R, 9aR)-6-Benzhydryl-8-[2,6-dimethoxy-3-(5-(trifluoromethyl)-1H-tetrazol-1-yl]benzyl]octahydro-2H-pyrazino[1,2-a]pyrazin-2-yl]-2-oxoethanol dihydrochloride

mp: 128-132° C. IR (KBr) : 3402, 1653, 1599, 1496, 1448, 1238, 1169, 1101 cm⁻¹ NMR (DMSO-d₆, δ): 3.31 (2H, s), 3.78 (3H, s), 4.05 (3H, s), 2.20-4.80 (16H, m), 7.05-7.50 (11H, m), 7.84 (1H, d, J=9.0 Hz) MASS (API-ES): 652 (M+H)⁺ (free)

### (5) (6R,9aR)-6-Benzhydryl-8-[2,6-dimethoxy-3-[5-(trifluoromethyl)-1H-tetrazol-1-yl]benzyl]-N,N-dimethyloctahydro-2H-pyrazino[1,2-a]pyrazine-2-carboxamide dihydrochloride

NMR (DMSO-d₆, δ): 2.30-4.60 (27H, m), 7.07-7.44 (11H, m), 7.84 (1H, d, J=9.0 Hz) MASS (APCI+): 665.13 M+H)⁺

### (6) 2-[(6R, 9aR)-6-Benzhydryl-8-[[2,4-dimethoxy-5-[5-(trifluoromethyl)-1H-tetrazol-1-yl]-3-pyridyl]methyl)-octahydro-2H-pyrazino [1,2-a]pyrazin-2-yl]-2-oxoethanol dihydrochloride

NMR (CDCl₃, δ) (free form): 1.81-1.98 (1H, m), 1.99-2.18 (2H, m), 2.30-2.48 (2H, m), 2.52-2.77 (2H, m), 2.82-3.05 (2H, m), 3.05-3.26 (2H, m), 3.40-3.52 (3H, m), 3.55 (3H, s), 3.82 and 3.86 (total 3H, each s), 3.96-4.26 (4H, m), 7.16-7.30 (10H, m), 8.04 and 8.06 (total 1H, each s) MASS (API-ES): 653 (M+H)⁺ (free)

### (7) 2-[(6R, 9aR)-6-Benzhydryl-8-[2,6-diethoxy-3-[5-trifluoroethyl]-1H-tetrazol-1-yl]benzyl)octahydro-2H-pyrazino [1,2-a]pyrazin-2-yl]-2-oxoethanol dihydrochloride

NMR (CDCl₃, δ)(free form): 0.94 (3H, t, J=7.0 Hz), 1.35 (3H, t, J=7.0 Hz), 1.75-3.20 (11H, m), 3.49-4.20 (11H, m), 6.63-6.70 (1H, m), 7.11-7.29 (11H, m) MASS (API-ES): 680 (M+H)⁺

### (8) 2-[(6R, 9aR)-6-Benzhydryl-8-[6-methoxy-2-methyl-3-[5-(trifluoromethyl)-1H-tetrazol-1-yl]benzyl]octahydro-2H-pyrazino[1,2-a]pyrazin-2-yl)-2-oxoethanol dihydrochloride

NMR (DMSO-d₆, δ): 1.91-1.99 (3H, m), 2.20-4.40 (20H, m), 7.09-7.41 (11H, m), 7.75 (1H, d, J=8.9 Hz) MASS (APCI+): 636.8 (M+H)⁺ (free)

### (9) 2-[(6R, 9aR)-6-Benzhydryl-8-[3-chloro-2,6-dimethoxy-5-[5-(trifluoromethyl)-1H-tetrazol-1-yl]benzyl]octahydro-2H-pyrazino[1,2-a]pyrazin-2-yl]-2-oxoethanol dihydrochloride

NMR (DMSO-d₆, δ): 2.20-4.70 (23H, m), 7.18-7.46 (10H, m), 8.09 (1H, d, J=3.4 Hz) MASS (ESI+): 686.3 (M+H)⁺, 708.3 (M+Na) (free)

### (10) (4R, 8aS)-4-Benzhydryl-2-[2,6-dimethoxy-3-[5-(trifluoromethyl)-1H-tetrazol-1-yl) benzyl]octahydropyrrolo[1,2-a]pyrazine dihydrochloride

NMR (CDCl₃, δ) (free form): 1.22-1.82 (3H, m), 1.88-2.04 (2H, m), 2.18-2.39 (1H, m), 2.47 (1H, br d, J=10.7 Hz), 2.56-2.68 (1H, m), 2.92 (1H, br d, J=10.3 Hz), 3.23 (1H, br t, J=8.94 Hz), 3.50 (3H, s), 3.59 (3H, s), 3.45-3.69 (3H, m), 3.98-4.00 (2H, m), 6.64 (1H, d, J=8.96 Hz), 6.87-7.41 (11H, m) MASS (APCI): 579 (M+H)⁺

### (11) (4R, 7S, 8aS)-4-Benzhydryl-2-[2,6-dimethoxy-3-[5-(trifluoromethyl)-1H-tetrazol-1-yl]benzyl]octahydropyrrolo-[1,2-a]pyrazin-7-ol dihydrochloride

Free NMR (CDCl₃, δ): 1.72-2.35 (7H, m), 2.46-2.59 (2H, m), 2.90 (1H, d, J=10.7 Hz), 3.19 (1H, d, J=8.64 Hz), 3.50 (3H, s), 3.58 (2H, s), 3.62 (3H, s), 3.94-4.10 (2H, m), 6.67 (1H, d, J=8.92 Hz), 7.11-7.38 (11H, m) MASS (APCI): 595 (M+H)⁺

Dihydrochloride MASS (APCI): 595 (M+H)⁺

### (12) (4R,7R,8aS)-4-Benzhydryl-2-(2,6-dimethoxy-3-[5-(trifluoromethyl)-1H-tetrazol-1-yl]benzyl]octahydropyrrolo-[1,2-a)pyrazin-7-ol dihydrochloride

NMR (CDCl₃, δ)(free form): 1.41-1.70 (3H, m), 1.80-1.97 (3H, m), 2.43 (1H, br d, J=11.9 Hz), 2.72-2.90 (3H, m), 2.98 (1H, dd, J=9.94, 6.0 Hz), 3.40-3.57 (3H, m), 3.50 (3H, m), 3.63 (3H, m), 3.88 (1H, d, J=-9.82 Hz), 4.15 (1H, br s), 6.66 (1H, d, J=8.94 Hz), 7.08-7.40 (11H, m) MASS (APCI): 595 (M+H)⁺ (free)

### (13) (4R, 7S, 8aS)-4-Benzhydryl-2-[2,6-dimethoxy-3-[5-(trifluoromethyl)-1H-tetrazol-1-yl]benzyl]octahydropyrrolo-[1,2-a]pyrazine-7-carbonitrile dihydrochloride

NMR (CDCl₃, δ) (free form): 1.69-2.24 (5H, m), 2.39-2.48 (2H, m), 2.62-2.91 (3H, m), 3.21-3.38 (1H, m), 3.49 (3H, s), 3.56 (2H, s), 3.69 (3H, m), 3.95 (1H, d, J=9.96 Hz), 6.68 (1H, d, J=8.94 Hz), 7.09-7.40 (11H, m) MASS (API-ES) : 604 (M+H)⁺ (free)

### (14) N-[(4R, 7R, 8aS)-4-Benzhydryl-2-[2,6-dimethoxy-3-[5-(trifluoromethyl)-1H-tetrazol-1-yl]benzyl]octahydropyrrolo-[1,2-a]pyrazin-7-yl]acetamide dihydrochloride

NMR (DMSO-d₆, δ): 1.70 (3H, s), 1.80-4.80 (20H, m), 7.05 (1H, d, J=9.1 Hz), 7.08-7.53 (10H, m), 7.81 (1H, d, J=9.1 Hz) MASS (APCI+) : 636.07 (M+H)⁺

### (15) Benzyl (6R,9aR)-6-benzhydryl-8-[2,6-dimethoxy-3-[5-(trifluoromethyl)-1H-tetrazol-1-yl]benzyl)octahydro-2H-pyrazino[1,2-a]pyrazine-2-carboxylate

IR (KBr): 3431, 3402, 1705, 1697, 1498, 1456, 1165, 1099 cm⁻¹ NMR (CDCl₃, δ): 1.70-2.14 (3H, m), 2.22-4.24 (12H, m), 3.50 (3H, s), 3.66 (3H, s), 5.08 (2H, s), 6.67 (1H, d, J=8.9 Hz), 7.05-7.44 (16H, m) MASS (ES+): 750 (M+Na)⁺, 728 (M+H)⁺

### EXAMPLE 53

The following compound was obtained according to a similar manner to that of Preparation 33.

### (4R, 9aR)-4-Benzhydryl-2-[2,6-dimethoxy-3-[5-(trifluoromethyl)-1H-tetrazol-1-yl]benzyl]-8-(methylsulfonyl)octahydro-2H-pyrazino[1,2-a]pyrazine dihydrochloride

NMR (DMSO-d₆, δ): 2.30-4.50 (24H, m), 7.12-7.38 (11H, m), 7.86 (1H, d, J=9.0 Hz) MASS (ESI+): 672 (M+H)(free)

### EXAMPLE 54

The following compounds were obtained according to a similar manner to that of Example 17.

### (1) (4R, 9aR)-4-Benzhydryl-2-[2,6-dimethoxy-3-[5-(trifluoromethyl)-1H-tetrazol-1-yl]benzyl]-8-(3-methoxypropanoyl)octahydro-2H-pyrazino[1,2-a]pyrazine dihydrochloride

NMR (DMSO-d₆, δ): 2.20-4.50 (20H, m), 3.19 (3H, s), 3.32 (3H, S), 3.51 (2H, t, J=6.5 Hz), 7.08-7.41 (11H, m), 7.84 (1H, d, J=9.0 Hz) MASS (ESI+): 680 (M+H), 702 M+Na) (free)

### (2) (4R, 9aR)-4-Benzhydryl-2-[2,6-dimethoxy-3-[5-(trifluoromethyl)-1H-tetrazol-1-yl]benzyl]-8-(methoxyacetyl)octahydro-2H-pyrazino[1,2-a]pyrazine dihydrochloride

NMR (DMSO-d₆, δ): 2.20-4.50 (26H, m), 7.12-7.87 (12H, m) MASS (ESI+): 666.07 (M+H)⁺ (free)

### (3) (4R, 9aR)-4-Benzhydryl-2-[2,6-dimethoxy-3-[5-(trifluromethyl)-1H-tetrazol-1-yl]benzyl]-8-(ethoxyacetyl)octahydro-2H-pyrazino[1,2-a]pyrazine dihydrochloride

NMR (DMSO-d₆, δ): 1.09 (3H, t, J=6.4 Hz), 2.30-4.50 (25H, m), 7.08-7.41 (11H, m), 7.85 (1H, d, J=9.0 Hz) MASS (ESI+): 680 (M+H)⁺ (free)

### (4) 4R,9aR)-4-Benzhydryl-2-[2,6-dimethoxy-3-[5-(trifluoromethyl)-1H-tetrazol-1-yl]benzyl]-8-(2-pyrazinylcarbonyl)octahydro-2H-pyrazino[1,2-a]pyrazine trihydrochloride

NMR (DMSO-d₆, δ): 2.20-4.50 (21H, m), 7.10-7.38 (11H, m), 7.85 (1H, d, J=8.6 Hz), 8.63-8.85 (3H, m) MASS (ESI+): 700 (M+H), 722 (M+Na) (free)

### (5) (4R,9aR)-4-Benzhydryl-2-[2,6-dimethoxy-3-[5-(trifluoromethyl)-1H-tetrazol-1-yl]benzyl]-8-(5-pyrimidinylcarbonyl)octahydro-2H-pyrazino[1,2-a]pyrazine trihydrochloride

NMR (DMSO-d₆, δ): 2.20-4.50 (21H, m), 7.09-7.40 (11H, m), 7.85 (1H, d, J=9.2 Hz), 8.86 (2H, s), 9.27 (1H, s) MASS (ESI+): 700 (M+H), 722 (M+Na) (free)

### (6) (4R,9aR)-4-Benzhydryl-2-[2,6-dimethoxy-3-[5-(trifluoromethyl)-1H-tetrazol-1-yl]benzyl]-8-(2-pyrimidinylcarbonyl)octahydro-2H-pyrazino[1,2-a]pyrazine trihydrochloride

NMR (DMSO-d₆, δ): 2.10-4.30 (21H, m), 7.11-7.45 (11H, m), 7.57-7.61 (1H, m), 7.83-7.89 (1H, m), 8.86 (2H, d, J=4.9 Hz) MASS (ESI+): 700 (M+H), 722 (M+Na) (free)

### (7) (4R,9aR)-4-Benzhydryl-2-[2,6-dimethoxy-3-[5-(trifluoromethyl)-1H-tetrazol-1-yl]benzyl]-8-[(1-methyl-1H-imidazol-2-yl)carbonyl]octahydro-2H-pyrazino[1,2-a]pyrazine trihydrochloride

NMR (DMSO-d₆, δ): 2.30-4.50 (24H, m), 7.08-7.64 (13H, m), 7.84 (1H, d, J=9.1 Hz) MASS (ESI+): 702 (M+H)⁺, 724 1+Na) (free)

### (8) (4R, 9aR)-4-Benzhydryl-2-[2,6-dimethoxy-3-[5-(trifluoromethyl)-1H-tetrazol-1-yl]benzyl]-8-(4-pyrimidinylcarbonyl)octahydro-2H-pyrazino[1,2-a)pyrazine trihydrochloride

NMR (DMSO-d₆, δ): 2.20-4.40 (21H, m), 7.11-7.41 (11H, m), 7.62-7.69 (1H, m), 7.86 (1H, d, J=9.2 Hz), 8.97 (1H, d, J=5.0 Hz), 9.39 (1H, s) MASS (ESI+): 700 (M+H), 722 (M+Na) (free)

### EXAMPLE 55

The following compound was obtained according to a similar manner to that of Preparation 31.

### (4R, 9aR)-4-Benzhydryl-8-(cyclobutylcarbonyl)-2-(2,6-dimethoxy-3-[5-(trifluoromethyl)-1H-tetrazol-1-yl]benzyl]octahydro-2H-pyrazino[1,2-a]pyrazine dihydrochloride

NMR (DMSO-d₆, δ): 1.60-4.50 (28H, m), 7.09-7.40 (11H, m), 7.85 (1H, d, J=9.0 Hz) MASS (ESI+): 676 (M+H) (free)

### EXAMPLE 56

The following compound was obtained according to a similar manner to that of Example 32.

### 1-[(6R, 9aR)-6-Benzhydryl-8-[2,6-dimethoxy-3-[5-(trifluoromethyl)-1H-tetazol-1-yl]benzyl]octahydro-2H-pyrazino[1,2-a]pyrazin-2-yl]acetone trihydrochloride

NMR (DMSO-d₆, δ): 2.15 (3H, s), 2.30-4.60 (23H, m), 7.11-7.31 (11H, m), 7.88 (1H, d, J=9.0 Hz) MASS (ESI+): 650 (M+H) (free)

### EXAMPLE 57

The following compounds were obtained according to a similar manner to that of Preparation 28.

### (1) (2R)-2-Benzhydryl-4-[2,6-dimethoxy-3-[5-(trifluoromethyl)-1H-tetrazol-1-yl]benzyl]-1-[(1-methyl-1H-pyrazol-4-yl)methyl]piperazine dihydrochloride

NMR (DMSO-d₆, δ): 2.20-5.30 (21H, m), 6.80-7.80 (14H, m) MASS (APCI): 633 (M+H)⁺ (free)

### (2) (2R)-2-Benzhydryl-4-[2,6-dimethoxy-3-[5-(trifluoromethyl)-1H-tetrazol-1-yl]benzyl]-1-(3-pyridylmethyl)piperazine trihydrochloride

NMR (DMSO-₆, δ): 2.20-5.00 (18H, m), 7.10-8.60 (16H, m) MASS (APCI): 630 (M+H)⁺ (free)

### EXAMPLE 58

The following compound was obtained according to a similar manner to that of Preparation 8.

### (4R, 8aS)-4-Benzhydryl-2-[2,6-dimethoxy-3-(5-(trifluoromethyl)-1H-tetrazol-1-yl]benzyl]hexahydropyrrolo[1,2-a]pyrazin-7(6H)-on dihydrochloride

Free form NMR (CDCl₃, δ): 1.92-2.59 (6H, m), 2.87-3.05 (3H, m), 3.50 (3H, s), 3.59 (2H, s), 3.66 (3H, s), 3.87 (1H, d, J=9.52 Hz), 6.70 (1H, d, J=8.92 Hz), 7.15-7.38 (11H, m) MASS (APCI): 593 (M+H)⁺

Dihydrochloride MASS (APCI) : 593 (M+H)⁺

### EXAMPLE 59

A solution of benzyl (6R,9aR)-6-benzhydryl-8-[2,6-dimethoxy-3-[5-(trifluoromethyl)-1H-tetrazol-1-yl]benzyl]octahydro-2H-pyrazino[1,2-a]pyrazine-2-carboxylate (2.26 g) and triethylamine (0.87 ml) in tetrahydrofuran (34 ml) was hydrogenated over 10% palladium on carbon (50% wet, 0.52 g) under atmospheric pressure at room temperature for 2 hours. The mixture was filtrated and the filtrate was evaporated in vacuo. The residue was purified by column chromatography on silica gel with chloroform:methanol:ammonia (20:1:0.1) as eluent to give (4R,9aR)-4-benzhydryl-2-[2,6-dimethoxy-3-[5-(trifluoromethyl)-1H-tetrazol-1-yl]benzyl)octahydro-2H-pyrazino[1,2-a]pyrazine (1.738 g).

IR (KBr): 3438, 1597, 1533, 1496, 1198, 1167, 1097 cm⁻¹ NMR (CDCl₃, δ): 1.66-3.60 (14H, m), 3.50 (3H, s), 3.64 (3H, s), 4.19 (1H, d, J=7.1 Hz), 6.66 (1H, d, J=8.9 Hz), 7.05-7.35 (11H, m) MASS (APCI): 594 (M+H)⁺

### EXAMPLE 60

(4R, 9aS)-4-Benzhydryl-2-[2,6-dimethoxy-3-[5-(trifluoromethyl)-1H-tetrazol-1-yl]benzyl]octahydro-2H-pyrazino[1,2-a]pyrazine (31.1 mg) was dissolved in ethyl acetate (1 ml), then to the solution was added 4N hydrogen chloride in ethyl acetate (0.5 ml). The solution was evaporated and dried under reduced pressure at 50° C. for 5 hours to give (4R,9aS)-4-benzhydryl-2-[2,6-dimethoxy-3-[5-(trifluoromethyl)-1H-tetrazol-1-yl]benzyl]octahydro-2H-pyrazino-[1,2-a]pyrazine trihydrochloride (32.5 mg) as a solid.

NMR (DMSO-d₆, δ): 2.20-4.50 (21H, m), 7.12-7.32 (11H, m), 7.88 (1H, d, J=9.0 Hz) MASS (ESI+): 594 (M+H)⁺ (free)

### EXAMPLE 61

The following compound was obtained according to a similar manner to that of Preparation 27 followed by Preparation 1.

### (3R)-3-Benzhydryl-1-[2,6-dimethoxy-3-[5-(trifluoromethyl)-1H-tetrazol-1-yl]benzyl]piperazine dihydrochloride

NMR (DMSO-d₆, δ): 2.20-4.80 (10H, m), 3.37 (3H, s), 3.73 (3H, s), 7.07 (1H, d, J=9.0 Hz), 7.78 (1H, d, J=9.0 Hz), 7.20-7.52 (10H, m), 7.75-7.80 (1H, m), 9.75-10.10 (1H, m) MASS (APCI): 539 (M+H)⁺

1. A compound of the formula (I): 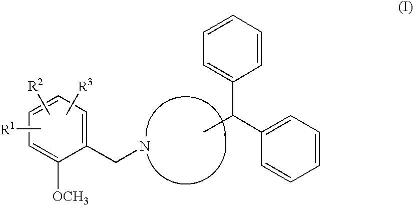
wherein 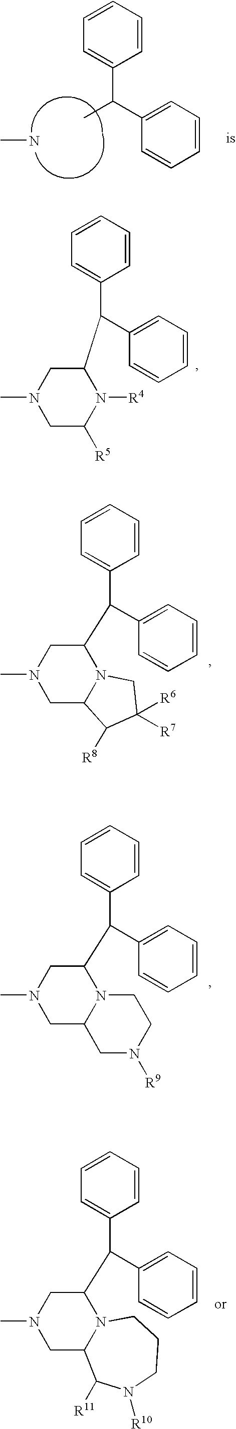 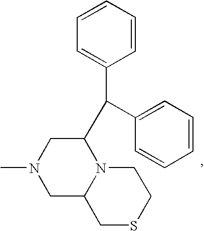
in which
R⁴ is hydrogen, lower alkanoyl or lower alkyl optionally substituted with carboxy, lower alkoxycarbonyl, pyridyl or lower alkylpyrazolyl,
R⁵ is hydrogen or lower alkoxycarbonyl,
R⁶ is hydrogen, halogen, oxo, hydroxy, lower alkanoyloxy, cyano, carbamoyl or amino optionally substituted with lower alkanoyl, hydroxy(lower)alkanoyl or benzyloxycarbonyl,
R⁷ is hydrogen or halogen,
R⁸ is hydrogen, oxo, lower alkanoyloxy, azido or amino optionally substituted with lower alkanoyl,
R⁹ is hydrogen; lower alkanoyl optionally substituted with hydroxy, carboxy, lower alkoxy, phenyl(lower)alkoxy, lower alkanoyloxy, lower alkoxycarbonyl, amino, lower alkanoylamino, benzyloxy(lower)alkanoylamino, hydroxy(lower)alkanoylamino, di(lower)alkylcarbamoyl or mono(or or )halogen(s); cyclo(lower)alkylcarbonyl optionally substituted with hydroxy(lower)alkyl, amino or lower alkanoylamino; azetidinylcarbonyl; lower alkylimidazolylcarbonyl; pyridylcarbonyl; pyrimidinylcarbonyl; pyrazinylcarbonyl; lower alkyl optionally substituted with imino, cyclo(lower)alkyl, lower alkanoyl, lower alkoxycarbonyl, carbamoyl or di(lower)alkylcarbamoyl; cyclo(lower)alkyl; carbamoyl optionally substituted with mono(or di)(lower)alkyl(s); aminosulfonyl optionally substituted with mono(or di)(lower)alkyl(s); lower alkylsulfonyl optionally substituted with hydroxy, lower alkylsulfonyl or lower alkanoyloxy; or pyridyl,
R¹⁰ is hydrogen or lower alkanoyl,
R¹¹ is hydrogen or oxo, and
R¹, R² and R³ are independently hydrogen, halogen, lower alkyl, lower alkoxy or tetrazolyl optionally substituted with mono(or di or tri)halo(lower)alkyl,
and a salt thereof.

2. The compound of claim 1, in which 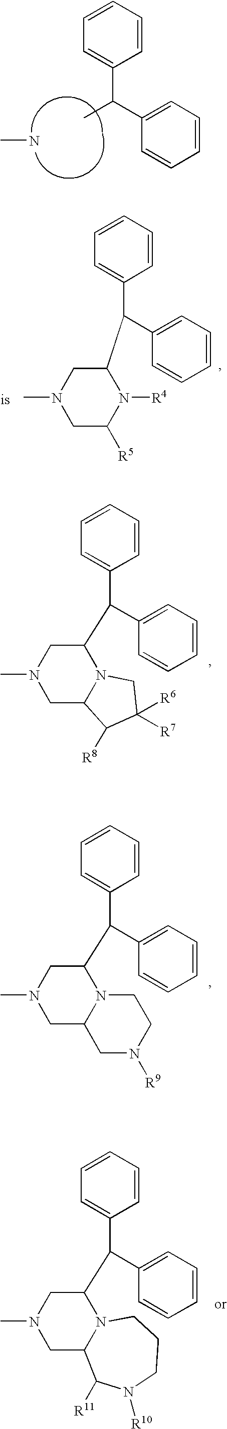 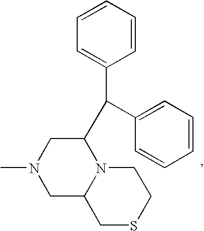
in which
R⁴ is lower alkanoyl or lower alkyl optionally substituted with carboxy, lower alkoxycarbonyl, pyridyl or lower alkylpyrazolyl,
R⁵ is hydrogen,
R⁶ is halogen, oxo, hydroxy, lower alkanoyloxy, cyano, carbamoyl or amino optionally substituted with lower alkanoyl, hydroxy(lower)alkanoyl or benzyloxycarbonyl,
R⁷ is hydrogen,
R⁸ is hydrogen,
R⁹ is lower alkanoyl substituted with hydroxy, carboxy, lower alkoxy, phenyl(lower)alkoxy, lower alkanoyloxy, lower alkoxycarbonyl, amino, lower alkanoylamino, benzyloxy(lower)alkanoylamino, hydroxy (lower) alkanoylamino, di(lower)alkylcarbamoyl or mono(or di or tri)halogen(s); cyclo(lower)alkylcarbonyl optionally substituted with hydroxy(lower)alkyl, amino or lower alkanoylamino; azetidinylcarbonyl; lower alkylimidazolylcarbonyl; pyridylcarbonyl; pyrimidinylcarbonyl; pyrazinylcarbonyl; lower alkyl optionally substituted with imino, cyclo(lower)alkyl, lower alkanoyl, lower alkoxycarbonyl, carbamoyl or di(lower)alkylcarbamoyl; cyclo(lower)alkyl; carbamoyl optionally substituted with mono(or di)(lower)alkyl(s); aminosulfonyl optionally substituted with mono(or di)(lower)alkyl(s); lower alkylsulfonyl optionally substituted with hydroxy, lower alkylsulfonyl or lower alkanoyloxy; or pyridyl,
R¹⁰ is hydrogen or lower alkanoyl,
R¹¹ is hydrogen or oxo, and
R¹, R² and R³ are independently hydrogen, halogen, lower alkyl, lower alkoxy or tetrazolyl optionally substituted with mono (or di or tri)halo(lower)alkyl.

3. The compound of claim 2, in which 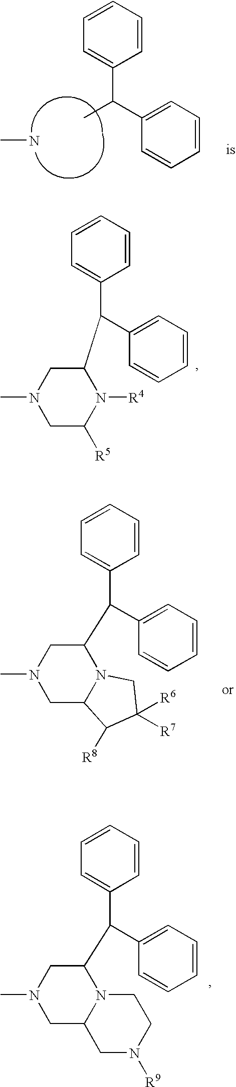
in which
R⁴ is lower alkanoyl or lower alkyl optionally substituted with carboxy, lower alkoxycarbonyl, pyridyl or lower alkylpyrazolyl,
R⁵ is hydrogen,
R⁶ is halogen, oxo, hydroxy, lower alkanoyloxy, cyano, carbamoyl or amino optionally substituted with lower alkanoyl, hydroxy(lower)alkanoyl or benzyloxycarbonyl,
R⁷ is hydrogen,
R⁸ is hydrogen,
R⁹ is lower alkanoyl substituted with hydroxy, carboxy, lower alkoxy, phenyl(lower)alkoxy, lower alkanoyloxy, lower alkoxycarbonyl, amino, lower alkanoylamino, benzyloxy(lower)alkanoylamino, hydroxy(lower)alkanoylamino, di(lower)alkylcarbamoyl or mono(or di or tri)halogen(s); cyclo(lower)alkylcarbonyl optionally substituted with hydroxy(lower)alkyl, amino or lower alkanoylamino; azetidinylcarbonyl; lower alkylimidazolylcarbonyl; pyridylcarbonyl; pyrimidinylcarbonyl; pyrazinylcarbonyl; lower alkyl optionally substituted with imino, cyclo(lower)alkyl, lower alkanoyl, lower alkoxycarbonyl, carbamoyl or di(lower)alkylcarbamoyl; cyclo(lower)alkyl; carbamoyl optionally substituted with mono(or di)(lower)alkyl(s); aminosulfonyl optionally substituted with mono(or di)(lower)alkyl(s); lower alkylsulfonyl optionally substituted with hydroxy, lower alkylsulfonyl or lower alkanoyloxy; or pyridyl, and
R¹, R² and R³ are independently hydrogen, lower alkoxy or tetrazolyl substituted with mono(or di or tri)halo(lower)alkyl.

4. A compound of claim 3, which is selected from a group consisting of
(1) (4R,9aR)-4-benzhydryl-2-[2,6-dimethoxy-3-[5-(trifluoromethyl)-1H-tetrazol-1-yl]benzyl]-8-(methoxyacetyl)octahydro-2H-pyrazino[1,2-a]pyrazine dihydrochloride,
(2) 2-[(6R, 9aR)-6-benzhydryl-8-[2,6-dimethoxy-3-(5-(trifluoromethyl)-1H-tetrazol-1-yl]benzyl)octahydro-2H-pyrazino[1,2-a]pyrazin-2-yl]-2-oxoethanol,
(3) (6R,9aR)-6-benzhydryl-8-[2,6-dimethoxy-3-[5-(trifluoromethyl)-1H-tetrazol-1-yl]benzyl]-N,N-dimethyloctahydro-2H-pyrazino[1,2-a]pyrazine-2-carboxamide,
(4) N-[(4R,7R,8aS)-4-benzhydryl-2-[2,6-dimethoxy-3-[5-(trifluoromethyl)-1H-tetrazol-1-yl]benzyl)-octahydropyrrolo[1,2-a]pyrazin-7-yl]acetamide, and
(5) (2R)-2-benzhydryl-4-[2,6-dimethoxy-3-[5-(trifluoromethyl)-1H-tetrazol-1-yl]benzyl]-1-[(1-methyl-1H-pyrazol-4-yl)methyl]piperazine,
or a pharmaceutically acceptable salt thereof.

5. A process for the preparation of the compound of claim 1, or a salt thereof, which comprises,
(1) reacting a compound of the formula (II): 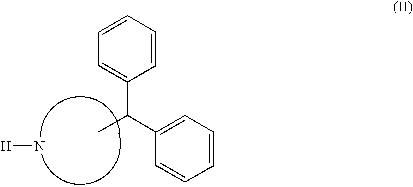
wherein 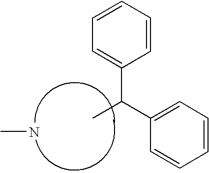
is as defined in claim 1, or its reactive derivative at the imino group or a salt thereof, with a compound of the formula (III): 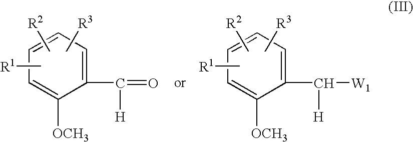
wherein R¹, R² and R³ are each as defined in claim 1, and
W₁ is a leaving group,
or a salt thereof to give a compound of the formula (I): 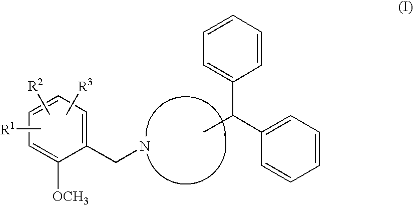
wherein 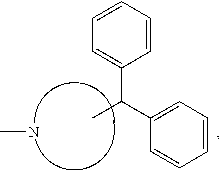
R¹, R² and R³ are each as defined in claim 1,
or a salt thereof, or
(2) reacting a compound of the formula (Ia): 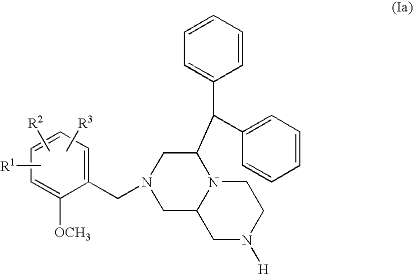
wherein R¹, R² and R³ are each as defined in claim 1, or a salt thereof, with a compound of the formula (IV):
W₂—R⁹ (IV)
wherein R⁹ is as defined in claim 1, and
W₂ is a leaving group,
or a salt thereof to give a compound of the formula (Ib): 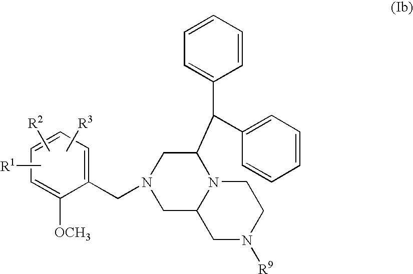
wherein R¹, R², R³ and R⁹ are each as defined in claim 1, or a salt thereof, or
(3) cyclizing a compound of the formula (V): 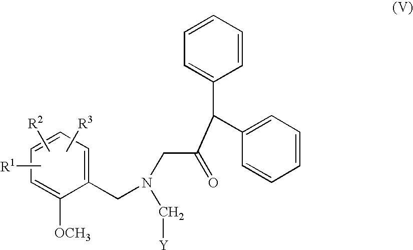
wherein R¹, R² and R³ are each as defined in claim 1, and
Y is 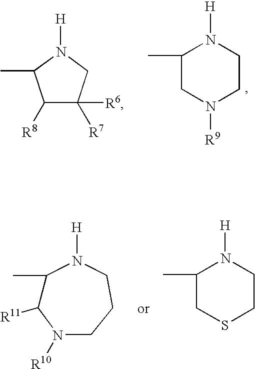
or its reactive derivative at the imino group or a salt thereof, to give a compound of the formula (Ic): 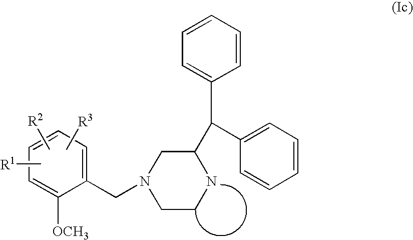
[in which 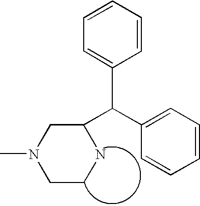
is 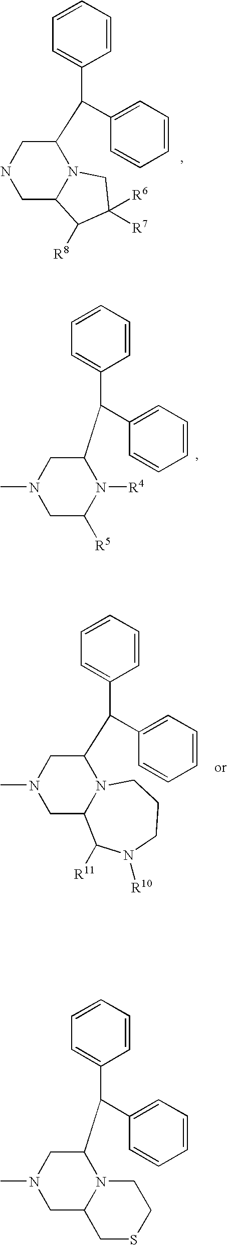
wherein R¹, R², R³, R⁶, R⁷, R⁸, R⁹, R¹⁰ and R¹¹ are each as defined in claim 1,
or a salt thereof, or
(4) reacting a compound of the formula (VI): 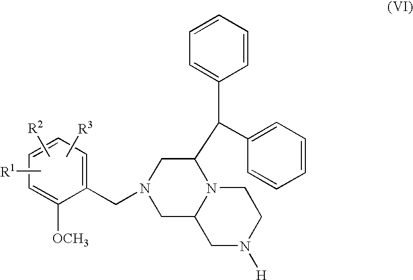
wherein R¹, R² and R³ are each as defined in claim 1, or its reactive derivative at the imino group or a salt thereof with a compound of the formula (VII): 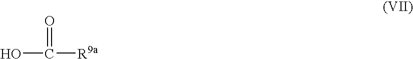
wherein R^{9a} is as defined in claim 1, to give a compound of the formula (Id): 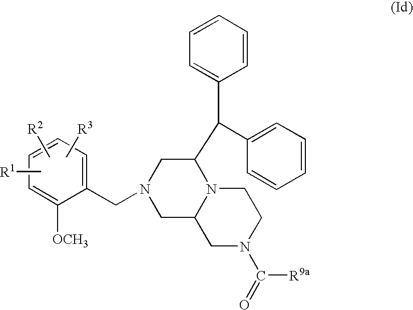
wherein R¹, R², R³ and R^{9a} are each as defined in claim 1,
or a salt thereof.

6. A pharmaceutical composition which comprises, as an active ingredient, a compound of claim 1 or a pharmaceutically acceptable salt thereof in admixture with pharmaceutically acceptable carriers.

7. A compound of claim 1 for use as a medicament.

8. A method for treating or preventing Tachykinin-mediated diseases which comprises administering an effective amount of a compound of claim 1 or a pharmaceutically acceptable salt thereof to human being or animals.

9. A compound of claim 1 for use as Tachykinin antagonist.

10. Use of a compound of claim 1 for manufacture of a medicament for treating or preventing Tachykinin-mediated diseases.
